(19) United States
(12) Patent Application Publication
(10) Pub. No.: US 2019/0374512 A1
(43) Pub. Date: Dec. 12, 2019

(54) COMPOUNDS FOR TREATMENT OF PANCREATIC CANCER

(71) Applicant: UNIVERSITY OF TENNESSEE RESEARCH FOUNDATION, Knoxville, TN (US)

(72) Inventors: WEI LI, Germantown, TN (US); Duane D. MILLER, Collierville, TN (US); Subhash CHAUHAN, Memphis, TN (US); Vivek Kumar KASHYAP, Memphis, TN (US); Wang QINGHUI, Memphis, TN (US)

(73) Assignee: UNIVERSITY OF TENNESSEE RESEARCH FOUNDATION, Knoxville, TN (US)

(21) Appl. No.: 16/413,222

(22) Filed: May 15, 2019

Related U.S. Application Data
(60) Provisional application No. 62/671,833, filed on May 15, 2018.

(51) Int. Cl. A61K 31/4178; A61P 35/00; A61K 31/427

(52) U.S. Cl. CPC A61K 31/4178; A61K 31/427; A61P 35/00

(57) ABSTRACT

The present invention encompasses methods of treating pancreatic cancer using therapeutically effective amounts of compounds represented by the structure of formula I.

This application claims the benefit of U.S. Provisional Application No. 62/671,833, filed May 15, 2018, hereby incorporated by reference.

### STATEMENT REGARDING FEDERALLY SUPPORTED RESEARCH OR DEVELOPMENT

The invention described herein was made with government support under Grant No. CA148706, awarded by The National Institutes of Health. The government has certain rights in the invention.

### FIELD OF THE INVENTION

The present invention relates to novel methods of treating pancreatic cancer by administering to a subject in need thereof a therapeutically effective amount of at least one compound of Formula I or a pharmaceutically acceptable salt thereof, optionally including a pharmaceutically acceptable excipient.

### BACKGROUND OF THE INVENTION

Cancer is the second most common cause of death in the United States, exceeded only by heart disease. In the United States, cancer accounts for 1 of every 4 deaths. The 5-year relative survival rate for all cancer patients diagnosed in 1996-2003 is 66%, up from 50% in 1975-1977 (Cancer Facts & Figures American Cancer Society: Atlanta, Ga. (2008)). This improvement in survival reflects progress in diagnosing at an earlier stage and improvements in treatment. Discovering highly effective anticancer agents with low toxicity is a primary goal of cancer research.

Microtubules are cytoskeletal filaments consisting of al-tubulin heterodimers and are involved in a wide range of cellular functions, including shape maintenance, vesicle transport, cell motility, and division. Tubulin is the major structural component of the microtubules and a well verified target for a variety of highly successful anti-cancer drugs. Compounds that interfere with microtubule-tubulin equilibrium in cells are effective in the treatment of cancers. Anticancer drugs like taxol and vinblastine interfere with microtubule-tubulin equilibrium in cells are extensively used in cancer chemotherapy. There are three major classes of antimitotic agents. Microtubule-stabilizing agents, which bind to fully formed microtubules and prevent the depolymerization of tubulin subunits, are represented by taxanes and epothilones. The other two classes of agents are microtubule-destabilizing agents, which bind to tubulin dimers and inhibit their polymerization into microtubules. Vina alkaloids such as vinblastine bind to the vinca site and represent one of these classes. Colchicine and colchicine-site binders interact at a distinct site on tubulin and define the third class of antimitotic agents.

Both the taxanes and vinca alkaloids are widely used to treat human cancers, while no colchicine-site binders are currently approved for cancer chemotherapy yet. However, colchicine binding agents like combretastatin A-4 (CA-4) and ABT-751, are now under clinical investigation as potential new chemotherapeutic agents (Luo et al., ABT-751, “A novel tubulin-binding agent, decreases tumor perfusion and disrupts tumor vasculature,” Anticancer Drugs, 2009, 20(6), 483-92; Mauer, et al., “A phase II study of ABT-751 in patients with advanced non-small cell lung cancer,” J. Thoroc. Oncol., 2008, 3(6), 631-6; Rustin, et al., “A Phase Ib trial of CA4P (combretastatin A-4 phosphate), carboplatin, and paclitaxel in patients with advanced cancer,” Br. J. Cancer, 2010, 102(9), 1355-60).

Unfortunately, microtubule-interacting anticancer drugs in clinical use share two major problems, resistance and neurotoxicity. A common mechanism of multidrug resistance (MDR), namely ATP binding cassette (ABC) transporter protein-mediated drug efflux, limits their efficacy (Green, et al., “Beta-Tubulin mutations in ovarian cancer using single strand conformation analysis-risk of false positive results from paraffin embedded tissues,” Cancer Letters, 2006, 236(1), 148-54; Wang, et al., “Paclitaxel resistance in cells with reduced beta-tubulin,” Biochimica et Biophysica Acta, Molecular Cell Research, 2005, 1744(2), 245-255; Leslie, et al., “Multidrug resistance proteins: role of P-glycoprotein, MRP1, MRP2, and BCRP (ABCG2) in tissue defense,” Toxicology and Applied Pharmacology, 2005, 204(3), 216-237).

P-glycoproteins (P-gp, encoded by the MDR1 gene) are important members of the ABC superfamily. P-gp prevents the intracellular accumulation of many cancer drugs by increasing their efflux out of cancer cells, as well as contributing to hepatic, renal, or intestinal clearance pathways. Attempts to co-administer P-gp modulators or inhibitors to increase cellular availability by blocking the actions of P-gp have met with limited success (Gottesman, et al., “The multidrug transporter, a double-edged sword,” J. Biol. Chem., 1988, 263(25), 12163-6; Fisher, et al., “Clinical studies with modulators of multidrug resistance,” Hematology/Oncology Clinics of North America, 1995, 9(2), 363-82).

The other major problem with taxanes, as with many biologically active natural products, is its lipophilicity and lack of solubility in aqueous systems. This leads to the use of emulsifiers like Cremophor EL and Tween 80 in clinical preparations. A number of biologic effects related to these drug formulation vehicles have been described, including acute hypersensitivity reactions and peripheral neuropathies (Hennenfent, et al., “Novel formulations of taxanes: a review. Old wine in a new bottle?” Ann. Oncol., 2006, 17(5), 735-49; Ten Tije, et al., “Pharmacological effects of formulation vehicles: implications for cancer chemotherapy,” Clin. Pharmacokinet., 2003, 42(7), 665-85).

Compared to compounds binding the paclitaxel- or vinca alkaloid binding site, colchicine-binding agents usually exhibit relatively simple structures. Thus, providing a better opportunity for oral bioavailability via structural optimization to improve solubility and pharmacokinetic (PK) parameters. In addition, many of these drugs appear to circumvent P-gp-mediated MDR. Therefore, these novel colchicine binding site targeted compounds hold great promise as therapeutic agents, particularly since they have improved aqueous solubility and overcome P-gp mediated MDR.

Pancreatic cancer is one of the most lethal cancers and ranked as the fourth most common cause of cancer-related deaths among both men and women in the United States. Siegel, et al., “Cancer statistics,” Cancer J. Clin., 2016, 66, 7-30. The management of pancreatic cancer is exceptionally difficult due to poor response to available therapeutic regimens. Ansari et al., “Update on the management of pancreatic cancer: surgery is not enough,” World J Gastroenterol 2015, 21, 3157-3165. Thus, the identification of newer, highly effective therapeutic agents with no or minimal toxicity is highly desirable for the improved management of pancreatic cancer.

With the rapidly rising incidence of pancreatic cancer, and the high resistance to current therapeutic agents, developing more effective drugs for treating such cancers that can effectively circumvent MDR will provide significant benefits to cancer patients.

### SUMMARY OF THE INVENTION

In one embodiment, the invention encompasses methods of treating pancreatic cancer in a subject by administering a therapeutically effective amount of a compound of Formula XI to the subject, wherein Formula XI is represented by:

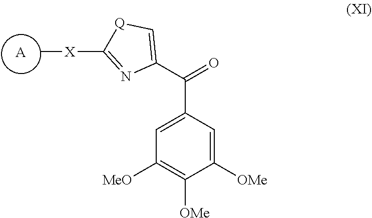

wherein
X is a bond, NH or S;
Q is O, NH or S; and
A is a ring and is substituted or unsubstituted saturated or unsaturated single-, fused- or multiple-ring, aryl or (hetero)cyclic ring system; N-heterocycle; S-heterocycle; O-heterocycle; cyclic hydrocarbon; or mixed heterocycle;
wherein the A ring is optionally substituted by 1-5 substituents which are independently O-alkyl, O-haloalkyl, F, Cl, Br, I, haloalkyl, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, alkylamino, aminoalkyl, —OCH₂Ph, —NHCO— alkyl, COOH, —C(O)Ph, C(O)O-alkyl, C(O)H, —C(O)NH₂ or NO₂;
i is an integer between 0-5;
wherein if Q is 5, then X is not a bond and pharmaceutically acceptable salts thereof.

Another embodiment of the invention encompasses methods of treating pancreatic cancer in a subject in need thereof by administering a therapeutically effective amount of a compound of Formula VIII to the subject, wherein Formula VIII is represented by the structure:

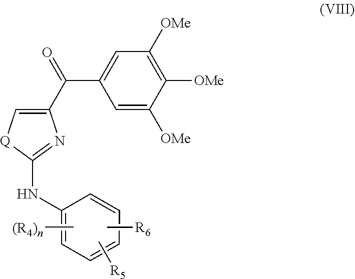

R₄, R₅ and R₆ each independently is hydrogen, O-alkyl, O-haloalkyl, F, Cl, Br, I, haloalkyl, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, alkylamino, aminoalkyl, —OCH₂Ph, —NHCO-alkyl, COOH, —C(O)Ph, C(O)O-alkyl, C(O)H, —C(O)NH₂ or NO₂;
Q is S, O or NH;
i is an integer between 0-5; and
n is an integer between 1-3 and pharmaceutically acceptable salts thereof.

Yet another embodiment, of the invention encompasses methods of treating pancreatic cancer in a subject in need thereof by administering a therapeutically effective amount of a compound of Formula XI(b) to the subject, wherein Formula XI(b) is represented by the structure:

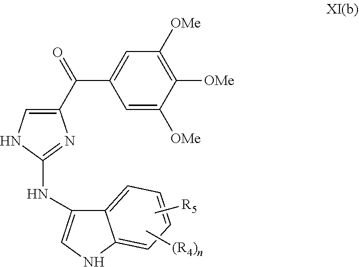

wherein R₄ and R₅ are independently hydrogen, O-alkyl, O-haloalkyl, F, Cl, Br, I, haloalkyl, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4 and pharmaceutically acceptable salts thereof.

One embodiment of the invention encompasses methods of treating pancreatic cancer in a subject in need thereof by administering a therapeutically effective amount of a compound of Formula XI(c) to the subject, wherein the compound of Formula XI(c) is represented by the structure:

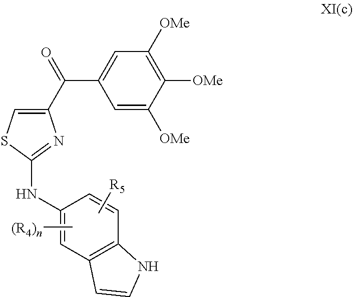

wherein R₄ and R₅ are independently hydrogen, O-alkyl, O-haloalkyl, F, Cl, Br, I, haloalkyl, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4 and pharmaceutically acceptable salts thereof.

Another embodiment of the invention encompasses methods of treating pancreatic cancer in a subject in need thereof by administering a compound of Formula XI(e), wherein Formula XI(e) is represented by the structure:

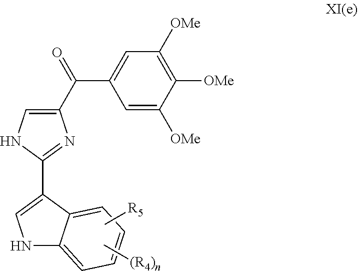

wherein R₄ and R₅ are independently hydrogen, O-alkyl, O-haloalkyl, F, Cl, Br, I, haloalkyl, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4 and pharmaceutically acceptable salts thereof.

Yet another embodiment of the invention encompasses methods of treating pancreatic cancer in a subject in need thereof by administering to the subject a therapeutically effective amount of at least one of the following compounds: (2-(phenylamino)thiazol-4-yl)(3,4,5-trimethoxyphenyl)methanone (5a), (2-(p-tolylamino)thiazol-4-yl)(3,4,5-trimethoxyphenyl)methanone (5b), (2-(p-fluorophenylamino)thiazol-4-yl)(3,4,5-trimethoxyphenyl)methanone (5c), (2-(4-chlorophenylamino)thiazol-4-yl)(3,4,5-trimethoxyphenyl)methanone (5d), (2-(phenylamino)-1H-imidazol-4-yl)(3,4,5-trimethoxyphenyl)methanone (5e), 2-(1H-indol-3-yl)-1H-imidazol-4-yl)(3,4,5-trimethoxyphenyl)methanone (17ya); and (2-(1H-indol-5-ylamino)thiazol-4-yl)(3,4,5-trimethoxyphenyl)methanone (55).

In another embodiment, the compound of this invention is its stereoisomer, pharmaceutically acceptable salt, hydrate, N-oxide, or combinations thereof. The invention includes pharmaceutical compositions comprising a compound of this invention and a pharmaceutically acceptable carrier.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A and 1B illustrate two graphs of the anti-cancer activity of Compound 17ya in vitro. FIG. 1A illustrates the IC₅₀ of Compound 17ya was 20, 30 and 40 nM in Panc-1, AsPC-1 and HPAF-II, respectively, after 24 hours of treatment. FIG. 1B illustrates the IC₅₀ of Compound 17ya was 8.2, 12.5, and 20 nM in Panc-1, AsPC-1 and HPAF-II, respectively, after 48 hours of treatment.

FIGS. 2A and 2B illustrate the growth inhibitory effect of Compound 17ya. FIG. 2A illustrates the growth curve, recorded as the basal cell index value, wherein Compound 17ya significantly reduced the cell index in a dose-dependent manner compared over the control (vehicle) in treating pancreatic cancer cells, Panc-1. FIG. 2B illustrates the growth curve, recorded as the basal cell index value (y axis is Cell Index), wherein Compound 17ya significantly reduced the cell index in a dose-dependent manner compared over the control (vehicle) in treating pancreatic cancer cells, AsPC-1

FIGS. 3A, 3B, and 3C illustrate the effect of Compound 17ya on the growth of pancreatic cancer cells. FIG. 3A illustrates the effect of Compound 17ya at 0, 1.25, 25, and 5 nM on pancreatic cancer cells Panc-1 in the colony formation and clonogenic potential (%) in graphic representation. FIG. 3B illustrates the effect of Compound 17ya at 0, 1.25, 2.5, and 5 nM on pancreatic cancer cells AsPC-1 in the colony formation and clonogenic potential (%) in graphic representation. FIG. 3C illustrates the effect of Compound 17ya at 0, 1.25, 2.5, and 5 nM on pancreatic cancer cells HPAF-II in the colony formation and clonogenic potential (%) in graphic representation.

FIGS. 4A and 4B illustrate the effect of Compound 17ya on the expression of βIII and βIV-tubulins in pancreatic cancer cells. FIG. 4A illustrates in graphical form the dose-dependent manner in which Compound 17ya (from 0 to 20 nM) inhibited mRNA expression of βI-tubulin, βIIa-tubulin, βIIb-tubulin, βIII-tubulin, βIVa-tubulin, βIVb-tubulin, βV-tubulin, and βVI-tubulin using pancreatic cancer cells Panc-1 and AsPC-1, as determined by qRT-PCR. FIG. 4B illustrates in western blot analysis of the dose-dependent manner in which Compound 17ya (from 0 to 20 nM) inhibited mRNA expression of βI-tubulin, βIIa-tubulin, βIIb-tubulin, βIII-tubulin, βIVa-tubulin, βIVb-tubulin, βV-tubulin, and βVI-tubulin using pancreatic cancer cells Panc-1 and AsPC-1.

FIGS. 5A and 5B illustrate the effect of Compound 17ya on pancreatic cancer cells Panc-1 in graphical form and by western blot analysis. FIG. 5A illustrates in graphical form the effect of Compound 17ya (ABI-231), cochicine, vinorelbine, and paclitaxel on βIII-tubulin mRNA (fold change) on pancreatic cancer cells Panc-1 as treated with 5-40 nM. FIG. 5B illustrates in western blot the protein levels after treatment with Compound 17ya, coichicine, vinorelbine, and paclitaxel on βIII-tubulin.

FIGS. 6A, 6B, and 6C illustrate cell inhibition growth of Compound 17ya, coichicine, vinorelbine, and paclitaxel on pancreatic cell lines Panc-1, AsPC-1, and HPAF-II, respectively. FIG. 6A illustrates the cell inhibition growth by Compound 17ya, coichicine, and vinorelbine on pancreatic cells Panc-1, at 0, 5, 10, 20, and 40 nM.

FIG. 6B illustrates the cell inhibition growth by Compound 17ya, coichicine, and vinorelbine on pancreatic cells AsPC-1, at 0, 5, 10, 20, and 40 nM. FIG. 6C illustrates the cell inhibition growth by Compound 17ya, coichicine, and vinorelbine on pancreatic cells HPAF-II, at 0, 5, 10, 20, and 40 nM.

FIGS. 7A, 7B, and 7C illustrate effect of Compound 17ya on the expression of miR-200c in pancreatic cell lines Panc-1 and AsPC-1. FIG. 7A illustrates in graphical form the effect of Compound on 17ya on miR-200C (fold change) on pancreatic cell lines Panc-1, AsPC-1, and HPAF-II at 0, 5, 10, and 20 nM. FIG. 7B illustrates in graphical form the inhibition of miR-200c on the effect of Compound 17ya on expression of βIII-tubulin, which was rescued by transfecting the cells with miR-200c inhibitor. FIG. 7C illustrates the effect on protein of Compound 17ya and miR-200c mimic transfection of Panc-1 cells.

FIGS. 8A and 8B illustrates the effect of Compound 17ya on the migration of pancreatic cancer cells. FIG. 8A illustrates via healing wound graphs the inhibition by Compound 17ya on migration of pancreatic cells Panc-1 at 0, 1.25, and 2.5 nM. FIG. 8B illustrates via healing wound graphs the inhibition by Compound 17ya on migration of pancreatic cells AsPC-1 at 0, 1.25, and 2.5 nM.

FIGS. 9A and 9B illustrate the effect of Compound 17ya on pancreatic cell migration by transwell assay. FIG. 9A illustrates that Compound 17ya showed significant inhibition of Panc-1 and AsPC-1 pancreatic cells in a dose dependent manner (0, 1.25, and 2.5 nM). FIG. 9B illustrates in graphical form the same data on inhibition of cell migration of pancreatic cell lines Panc-1 and AsPC-1.

FIGS. 10A and 10B illustrate the effect of Compound 17ya on the migration and invasion of pancreatic cell lines Panc-1 at sublethal levels. FIG. 10A illustrates that Compound 17ya showed significant inhibition of Panc-1 and AsPC-1 pancreatic cell lines at sub lethal concentrations. FIG. 10B illustrates in graphical form the same data on inhibition of cell migration of pancreatic cell lines Panc-1 and AsPC-1.

FIGS. 11A and 11B illustrate graphs of cell migration and cell invasion as cell index over time (hours). FIG. 11A illustrates the effect of Compound 17ya at 5, 10, and 20 nM as compared to the control on cell migration on pancreatic cells Panc-1. FIG. 11B illustrates the effect of Compound 17ya at 5, 10, and 20 nM as compared to the control on cell invasion on pancreatic cells Panc-1.

FIGS. 12A, 12B, 12C, 12D, and 12E illustrate the effect of Compound 17ya on the cell cycle and its distribution and induced apoptosis in pancreatic cancer cells. FIG. 12A illustrates that Compound 17ya arrested the cell cycle of pancreatic cells Panc-1 at 5 nM, 10 nM, and 20 nM. FIG. 12B illustrates, using Western blot, that Compound 17ya in a dose dependent manner inhibit the protein levels of cyclin B1 and cdc25c in Panc-1 and AsPC-1 cells. FIG. 12C illustrates the effect of Compound 17ya on apoptosis induction in pancreatic cancer cells (Panc-1) by Annexin V-7AAD staining and mitochondrial membrane potential using flow cytometer. FIG. 12D illustrates, using Western blot, that Compound 17ya in a dose dependent manner inhibit the protein levels of cyclin B1 and cdc25c in Panc-1 and AsPC-1 cells. FIG. 12E illustrates a dose-dependent (5-20 nM) decrease of TMRE staining in pancreatic cells Panc-1 and illustrates the data in graphical form.

FIGS. 13A, 13B, 13C, 13D, 13E, 13F, 13G, 13H, and 13I illustrate the effective inhibition of pancreatic tumor growth in a xenograft mouse model. FIG. 13A illustrates the comparison between control and Compound 17ya in the reduction of tumor size. FIG. 13B illustrates the graphical representation in the reduction of tumor size and growth of the control as compared to Compound 17ya (50 nM). FIG. 13C illustrates the graphical representation in the reduction of tumor weight of the control as compared to Compound 17ya (50 nM). FIG. 13D illustrates the IHC results of the effective inhibition of PCNA expression by Compound 17ya as compared to the control as shown by immunohistochemistry. FIG. 13E illustrates the Western blot comparison of Compound 17ya and control with various tubulin. FIG. 13F illustrates the effect of Compound 17ya at the mRNA expression of βIII and βIVb-tubulins in xeonograph tumor tissues. FIG. 13G illustrates the effect Compound 17ya has on tubulin expression as compared to the control. FIG. 13H illustrates in graphical form the effect of Compound 17ya on the miR-200c (fold expression) as compared to the control. FIG. 13I illustrates the in situ hybridization assays on the expression of miE-200c in excised tumors.

FIG. 14A-D illustrate VERU-111 (compound 17ya) inhibited pancreatic cancer. FIG. 14A(i-ii) illustrate the dose dependent effect of VERU-111 (compound 17ya) over cell lines Panc-1, AsPC-1, and HPAF-II as percent of cell viability. FIG. 14B(i-ii) illustrate the time dependent effect of VERU-111 (compound 17ya) at 5 nM, 10 nM, and 20 nM as comparted to a control. FIG. 14C illustrates the effect of VERU-111 (compound 17ya) at 1.25 nM, 2.5 nM, and 5 nM as comparted to a control with Panc-1 (FIG. C(i)), AsPC-1 (FIG. C(ii)), or HPAF-II (FIG. C(iii)) cell lines. FIG. 14D illustrates the effect of VERU-111 (compound 17ya) at 1.25 nM, 2.5 nM, and 5 nM as comparted to a control with Panc-1 (FIG. D(i)), AsPC-1 (FIG. D(ii)), or HPAF-II (FIG. D(iii)) cell lines in bar graph form.

FIG. 15 illustrates VERU-111 (compound 17ya) inhibited pancreatic cancer.

FIG. 16 illustrates VERU-111 (compound 17ya) in preclinical safety (less myelosuppression, less neurotoxicity, maintains body weight), where FIG. 16 illustrates the toxicity tests of liver weight and white blood count (WBC) in mice in the use of VERU-111 (3.3 mpk or 6.7 mpk) and VERU-112 (10 mpk and 30 mpk) as compared to a control and DTX (10 mpk and 20 mpk).

FIG. 17 illustrates VERU-111 (compound 17ya) in preclinical safety (less myelosuppression, less neurotoxicity, maintains body weight), where FIG. 17 illustrates the neurotoxicity tests (hot plate test at 5.-52.5° C. and the time require to lick the paw recorded as latency period for pain threshold) in mice in the use of VERU-111 (3.3 mpk or 6.7 mpk) and VERU-112 (10 mpk and 30 mpk) as compared to a control and DTX (10 mpk and 20 mpk).

FIG. 18 illustrates VERU-111 (compound 17ya) as antiproliferative and maintains body weight as contrasted to the lack of efficacy of docetaxel in PC-3/Txr tumors, VERU-111 (compound 17ya) was dosed orally and had >100% TGI without an effect on body weight.

FIG. 19 illustrates nonclinical results in assessment of blockade of HERG potassium channels stably expressed in HEK293 cells and central nervous system safety study in rats with an IC₂₀ of 9.23 nM and the oral administration of VERU-111 (compound 17ya) at doses up to and including 10 mg/kg was not associated with any adverse effects on neurobehavioral function in rats.

FIG. 20 illustrates VERU-111 (compound 17ya) nonclinical results in cardiovascular and respiratory evaluation study in beagle dogs where VERU-111 (compound 17ya) was administered as doses of 2, 4, and 8 mg/kg to dogs and did not produce mortality or effects on blood pressure, heart rate, or the evaluated electrocardiogram or respiratory parameters. Increases in body temperature (50.7° C. maximum change) were observed at all doses of VERU-111 (compound 17ya) from approximately 3.5 to 11 hours post dose. Vomitus was noted between 4 and 24 hours following the 8 mg dose. Oral administration of VERU-111 (compound 17ya) at doses up to and including 8 mg/kg was not associated with any adverse effects on cardiovascular or respiratory function in dogs.

FIG. 21 illustrates VERU-111 (compound 17ya) pharmacokinetics in dogs were mean (±SD) and CV % for VERU-111 (compound 17ya) pharmacokinetic parameters on days 1 and 7 following oral capsule administration of 5 and 10 mg/kg VERU-111 to male gods.

FIG. 22 illustrates VERU-111 (compound 17ya) 28-day oral capsule toxicity study in beagle dogs that found that it did not impact dog survival, no ophthalmoscopic findings; no changes in hematology, coagulation, and urinalysis parameters; no clinical or macroscopic pathologic observations; at 4 and 8 mg/kg mild observations of inappetence, vomiting emesis, and diarrhea; dogs at 8 mg/kg/day had body weight losses; had QTc prolongation that exceeded 10% change; and reduced thymus organ weights and reduction of lymphocytes in thymus; no observed adverse effect level (NOAEL) was 4 mg/kg/day; and following 28 days of dose at 4 mg/kg/day the mean Cmax and AUC₀₋₁₂ₕᵣ values were 23.2 ng/ml and 71.7 hr*ng/mL, respectively.

FIGS. 23A and 23B illustrate VERU-111 (compound 17ya) 28-Day oral capsule toxicity study in dogs—weight. FIG. 23A illustrates the mean body weight in male dogs relative to time (weeks) from the start date. FIG. 23B illustrates the mean body weight in dogs relative to time (weeks) from the start date.

FIG. 24 illustrates VERU-111 (compound 17ya) 28-Day oral capsule toxicity study in dogs-QT interval.

FIG. 25 illustrates VERU-111 (compound 17ya) 28-Day oral capsule toxicity study in dogs-Hematology.

FIG. 26 illustrates VERU-111 (compound 17ya) 28-Day oral capsule toxicity study in dogs-Hematology.

FIG. 27 illustrates VERU-111 (compound 17ya) 28-Day oral capsule toxicity study in dogs-Liver function tests.

FIG. 28 illustrates VERU-111 (compound 17ya) 28-Day oral capsule toxicity study in dogs-Liver function tests.

FIG. 29A-B illustrate compound 17ya 28-day oral capsule toxicokinetics study in beagle dogs. FIG. 29A illustrates individual and mean compound 17ya C values on Days 1 and 28 following daily oral capsule administration of 2, 4, and 8 mg/kg compound 17ya to dogs (males and females combined). FIG. 29B illustrates individual and mean compound 17ya AUC₀₋₁₂ₕᵣ values on Days 1 and 28 following daily oral capsule administration of 2, 4, and 8 mg/kg compound 17ya to dogs (males and females combined).

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

The invention encompasses methods of treating pancreatic cancer by administering at least one compound of formula (I) in a therapeutically effective amount to a subject in need thereof, wherein the compound of Formula (I) has the formula

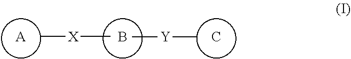

wherein
A and C are each independently substituted or unsubstituted single-, fused- or multiple-ring aryl or (hetero)cyclic ring systems; substituted or unsubstituted, saturated or unsaturated N-heterocycles; substituted or unsubstituted, saturated or unsaturated S-heterocycles; substituted or unsubstituted, saturated or unsaturated O-heterocycles; substituted or unsubstituted, saturated or unsaturated cyclic hydrocarbons; or substituted or unsubstituted, saturated or unsaturated mixed heterocycles;

### B is

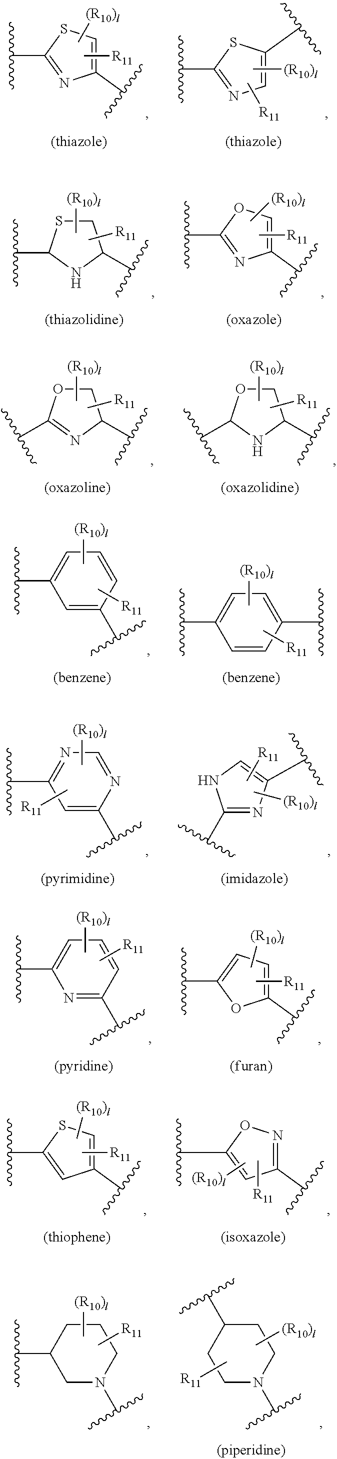

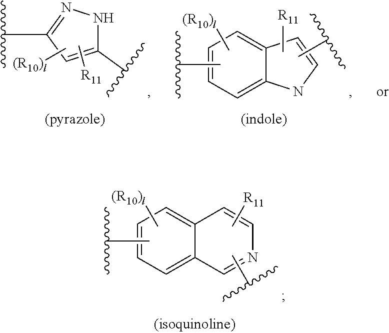

R₁₀ and R₁₁ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
X is a bond, NH, C₁ to C₅ hydrocarbon, O, or S;
Y is a bond, —C═O, —C═S, —C═N—NH₂, —C═N—OH, —CH—OH, —C═CH—CN, —C═N—CN, —CH═CH—, —C═C(CH₃)₂, —C═N—OMe, —(C═O)—NH, —NH—(C═O), —(C═O)—O, —O—(C═O), —(CH₂)₁₋₅—(C═O), (C═O)—(CH₂)₁₋₅, —(SO₂)—NH—, —NH—(SO₂)—, SO₂, SO or S;
wherein said A and C rings are optionally substituted by 1-5 substituents which are independently O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer between 0-5;
l in an integer between 0-2;
wherein
if B is a benzene ring, a thiophene ring, a furan ring or an indole ring then X is not a bond or CH₂, and A is not indole;
if B is indole then X is not O; and
or its pharmaceutically acceptable salt, hydrate, polymorph, metabolite, tautomer or isomer.

The pancreatic cancer may be taxane-resistance TNBC, taxane-sensitive TNBC, and/or metastasis.

In one embodiment, if B of formula I is a thiazole ring then X is not a bond.

In one embodiment, A in compound of Formula I is indolyl. In another embodiment A is 2-indolyl. In another embodiment A is phenyl. In another embodiment A is pyridyl. In another embodiment A is naphthyl. In another embodiment A is isoquinoline. In another embodiment, C in compound of Formula I is indolyl. In another embodiment C is 2-indolyl. In another embodiment C is 5-indolyl. In another embodiment, B in compound of Formula I is thiazole. In another embodiment, B in compound of Formula I is thiazole; Y is CO and X is a bond. Non limiting examples of compound of formula I are selected from: (2-(1H-Indol-2-yl)thiazol-4-yl)(1H-indol-2-yl)methanone (8) and (2-(1H-indol-2-yl)thiazol-4-yl)(1H-indol-5-yl)methanone (21).

The invention also encompasses a method of treating pancreatic cancer in a subject in need thereof by administering at least one compound of formula (Ia) in a therapeutically effective amount to the subject and wherein the compound of formula (Ia) has the structure

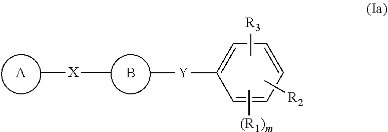

wherein
A is substituted or unsubstituted single-, fused- or multiple-ring, aryl or (hetero)cyclic ring systems; substituted or unsubstituted, saturated or unsaturated N-heterocycles; substituted or unsubstituted, saturated or unsaturated S-heterocycles; substituted or unsubstituted, saturated or unsaturated O-heterocycles; substituted or unsubstituted, saturated or unsaturated cyclic hydrocarbons; or substituted or unsubstituted, saturated or unsaturated mixed heterocycles;

### B is

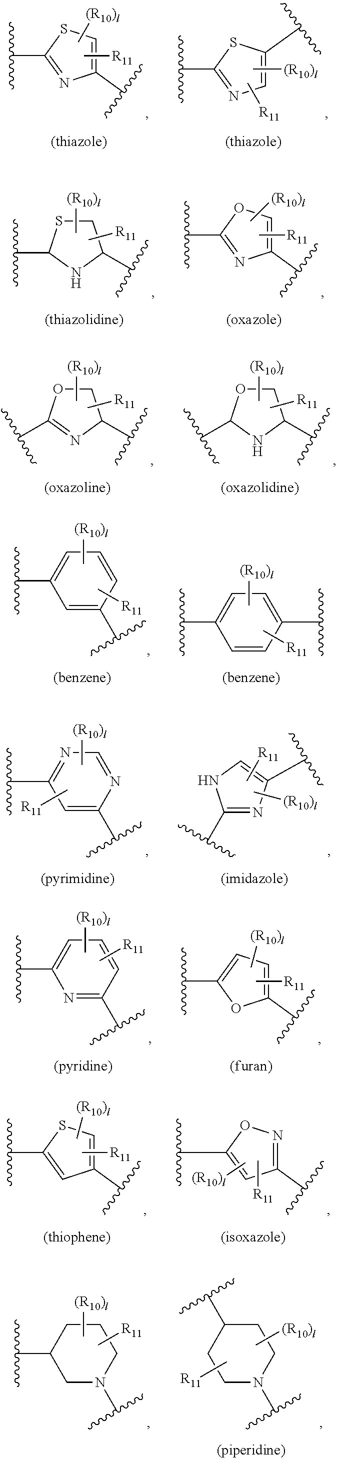

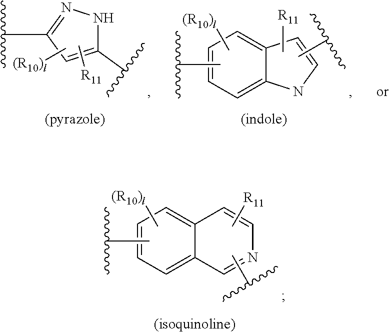

R₁, R₂ and R₃ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
R₁₀ and R₁₁ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
X is a bond, NH, C₁ to C₅ hydrocarbon, O, or S;
Y is a bond, —C═O, —C═S, —C═N—NH₂, —C═N—OH, —CH—OH, —C═CH—CN, —C═N—CN, —CH═CH—, —C═C(CH₃)₂, —C═N—OMe, —(C═O)—NH, —NH—(C═O), —(C═O)—O, —O—(C═O), —(CH₂)₁₋₅—(C═O), (C═O)—(CH₂)₁₋₅, —(SO₂)—NH—, —NH—(SO₂)—, SO₂, SO or S;
wherein said A ring is optionally substituted by 1-5 substituents which are independently O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer between 0-5;
l is an integer between 0-2;
m is an integer between 1-3;
wherein
if B is a benzene ring, a thiophene ring, a furan ring or an indole ring then X is not a bond or CH₂ and A is not indole;
if B is indole then X is not O;
or its pharmaceutically acceptable salt, hydrate, polymorph, metabolite, tautomer or isomer.

In one embodiment, if B of formula Ia is a thiazole ring then X is not a bond.

The invention also encompasses methods of treating pancreatic cancer by administering at least one compound of formula (II) in a therapeutically effective amount to a subject in need thereof, wherein the compound of Formula (II) has the formula:

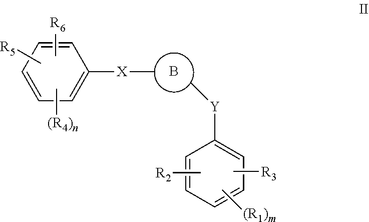

wherein

### B is

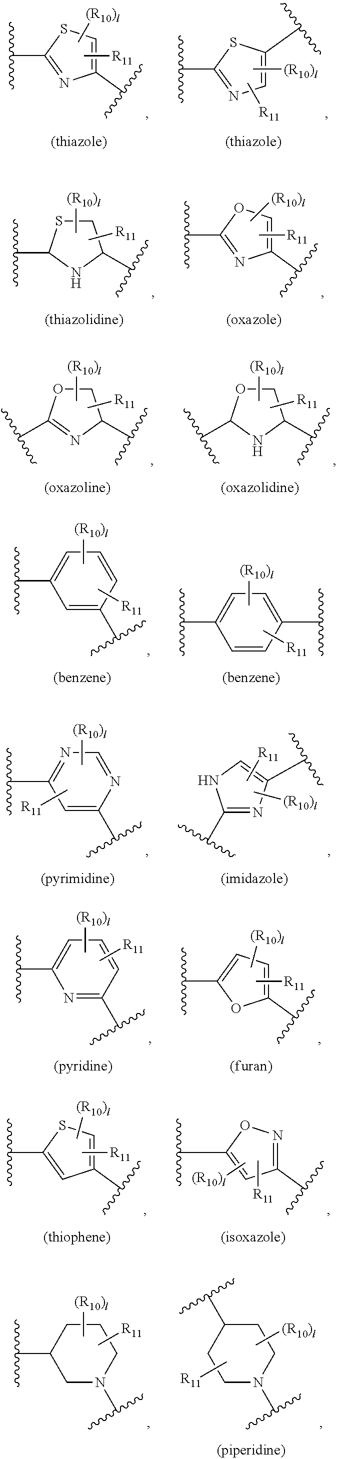

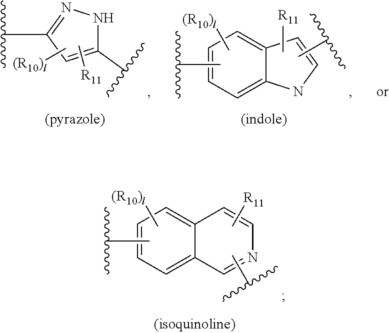

R₁, R₂, R₃, R₄, R₅ and R₆ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
R₁₀ and R₁₁ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
X is a bond, NH, C₁ to C₅ hydrocarbon, O, or S;
Y is a bond, —C═O, —C═S, —C═N—NH₂, —C═N—OH, —CH—OH, —C═CH—CN, —C═N—CN, —CH═CH—, C═C(CH₃)₂, —C═N—OMe, —(C═O)—NH, —NH—(C═O), —(C═O)—O, —O—(C═O), —(CH₂)₁₋₅—(C═O), (C═O)—(CH₂)₁₋₅, —(SO₂)—NH—, —NH—(SO₂)—, SO₂, SO or S;
i is an integer between 0-5;
l is an integer between 0-2;
n is an integer between 1-3; and
m is an integer between 1-3;
wherein
if B is indole then X is not O;
or its pharmaceutically acceptable salt, hydrate, polymorph, metabolite, tautomer or isomer.

In one embodiment, if B of formula II is a thiazole ring then X is not a bond.

The invention also encompasses methods of treating pancreatic cancer by administering at least one compound of formula (III) in a therapeutically effective amount to a subject in need thereof, wherein the compound of Formula (III) has the formula

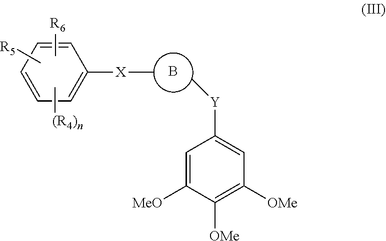

wherein

### B is

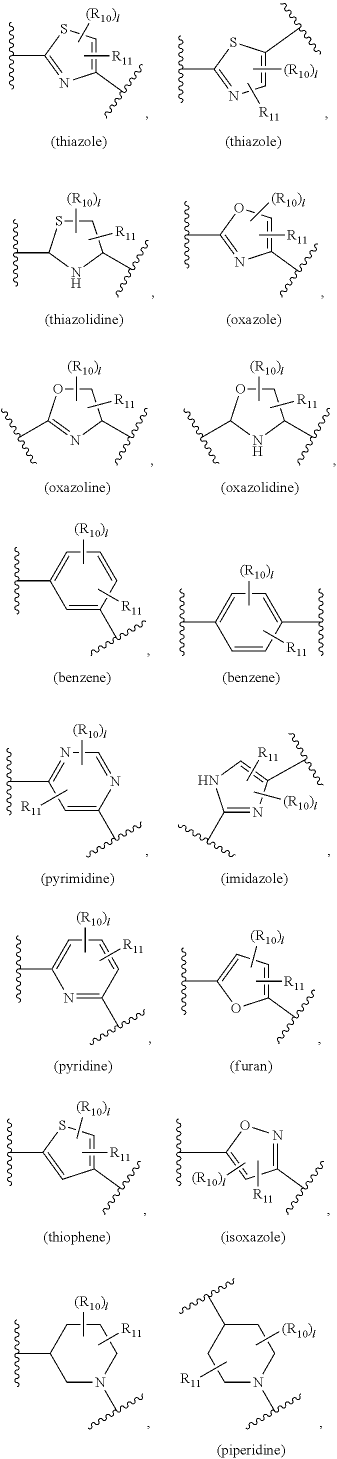

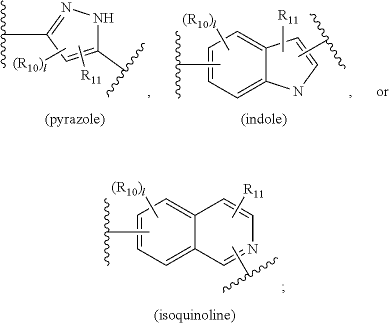

R₄, R₅ and R₆ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂; and
R₁₀ and R₁₁ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
X is a bond, NH, C₁ to C₅ hydrocarbon, O, or S;
Y is a bond, —C═O, —C═S, —C═N—NH₂, —C═N—OH, —CH—OH, —C═CH—CN, —C═N—CN, —CH═CH—, C═C(CH₃)₂, —C═N—OMe, —(C═O)—NH, —NH—(C═O), —(C═O)—O, —O—(C═O), —(CH₂)₁₋₅—(C═O), (C═O)—(CH₂)₁₋₅, —(SO₂)—NH—, —NH—(SO₂)—, SO₂, SO or S;
i is an integer between 0-5;
l is an integer between 0-2; and
n is an integer between 1-3;
wherein
if B is indole then X is not O;
or its pharmaceutically acceptable salt, hydrate, polymorph, metabolite, tautomer or isomer.

In one embodiment, if B of formula III is a thiazole ring then X is not a bond.

The invention encompasses methods of treating pancreatic cancer by administering at least one compound of formula (IV) in a therapeutically effective amount to a subject in need thereof, wherein the compound of Formula (IV) has the formula

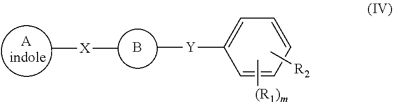

wherein ring A is an indolyl;

### B is

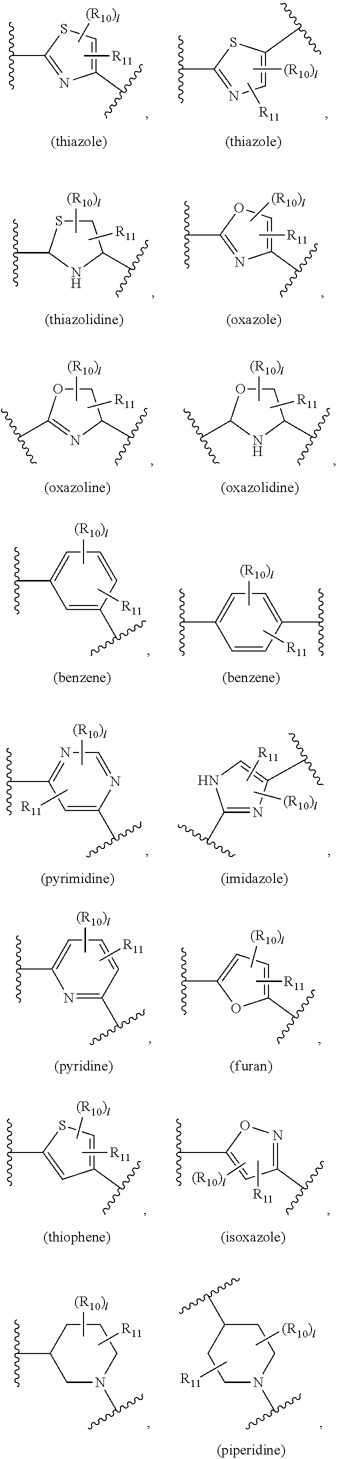

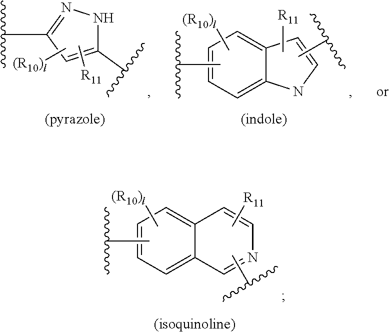

R₁ and R₂ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
R₁₀ and R₁₁ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
X is a bond, NH, C₁ to C₅ hydrocarbon, O, or S;
Y is a bond, C═O, —C═S, —C═N—NH₂, —C═N—OH, —CH—OH, —C═CH—CN, —C═N—CN, —CH═CH—, C═C(CH₃)₂, —C═N—OMe, —(C═O)—NH, —NH—(C═O), —(C═O)—O, —O—(C═O), —(CH₂)₁₋₅—(C═O), (C═O)—(CH₂)₁₋₅, —(SO₂)—NH—, —NH—(SO₂)—, SO₂, SO or S; wherein said A is optionally substituted by O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂; and
i is an integer between 0-5;
l is an integer between 0-2; and
m is an integer between 1-4;
wherein
if B is a benzene ring, a thiophene ring, a furan ring or an indole ring then X is not a bond or CH₂; or its pharmaceutically acceptable salt, hydrate, polymorph, metabolite, tautomer or isomer.

In one embodiment, if B of formula IV is a thiazole ring then X is not a bond.

In another embodiment, the indolyl of ring A of formula IV is attached to one of its 1-7 positions to X or direct to B if X is a bond (i.e., nothing).

The invention also encompasses methods of treating pancreatic cancer by administering at least one compound of formula IV(a) in a therapeutically effective amount to a subject in need thereof, wherein the compound of Formula IV(a) has the formula:

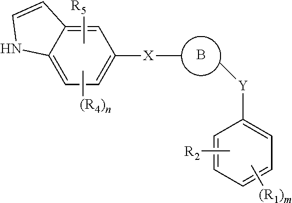

### B is

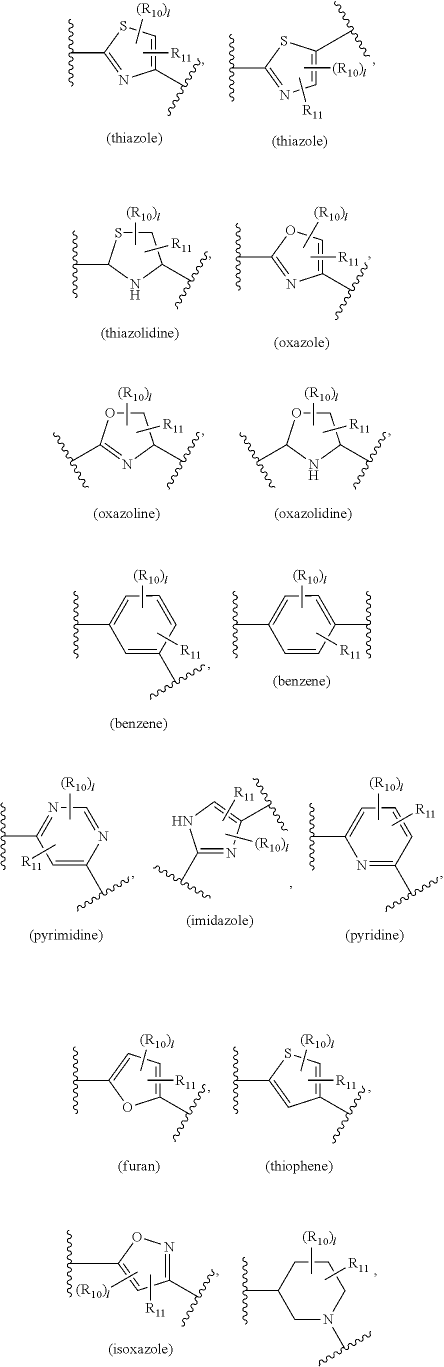

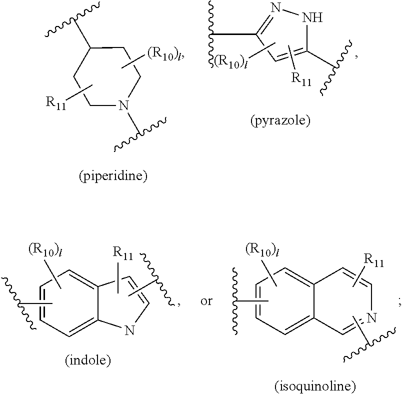

R₁, R₂, R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂; and
R₁₀ and R₁₁ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
X is a bond, NH, C₁ to C₅ hydrocarbon, O, or S;
Y is a bond or C═O, —C═S, —C═N—NH₂, —C═N—OH, —CH—OH, —C═CH—CN, —C═N—CN, —CH═CH—, C═C(CH₃)₂, —C═N—OMe, —(C═O)—NH, —NH—(C═O), —(C═O)—O, —O—(C═O), —(CH₂)₁₋₅—(C═O), (C═O)—(CH₂)₁₋₅, —(SO₂)—NH—, —NH—(SO₂)—, SO₂, SO or S;
i is an integer between 0-5;
l is an integer between 0-2;
n is an integer between 1-2; and
m is an integer between 1-4;
wherein
if B is a benzene ring, a thiophene ring, a furan ring or an indole ring then X is not a bond or CH₂; or its pharmaceutically acceptable salt, hydrate, polymorph, metabolite, tautomer or isomer.

In one embodiment, if B of formula IVa is a thiazole ring then X is not a bond.

The invention also encompasses methods of treating pancreatic cancer by administering at least one compound of formula (V) in a therapeutically effective amount to a subject in need thereof, wherein the compound of Formula (V) has the formula:

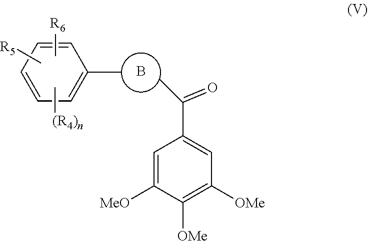

### B is

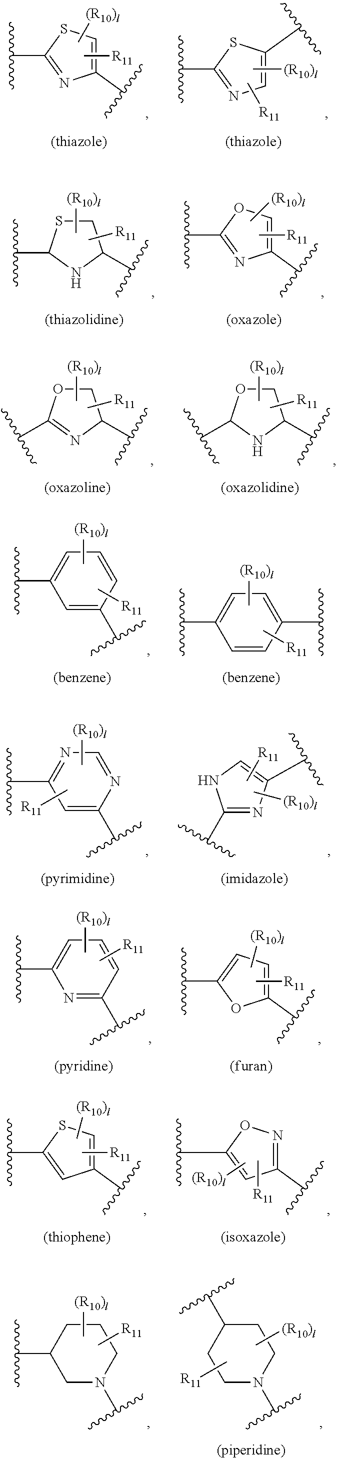

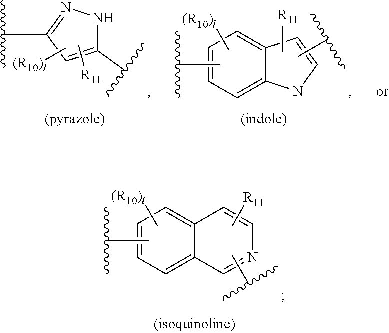

R₄, R₅ and R₆ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
R₁₀ and R₁₁ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer between 1-5;
l is an integer between 0-2; and
n is an integer between 1-3;
or its pharmaceutically acceptable salt, hydrate, polymorph, metabolite, tautomer or isomer.

In another embodiment, B of formula V is not a thiazole

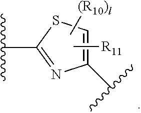

In another embodiment, B of formula V is not an oxazole. In another embodiment, B of formula V is not an oxazoline. In another embodiment, B of formula V is not an imidazole. In another embodiment, B of formula V is not a thiazole, oxazole, oxazoline or imidazole.

Compounds encompassed by the method of the invention include the following compounds:

| | | | | R₄, R₅ |
|---|---|---|---|---|
| | Compound | B | | and R₆ |
| 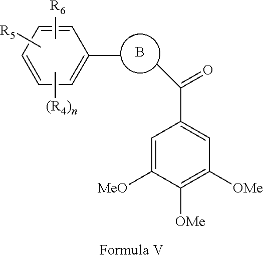 | 1a 1b | 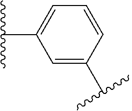 [SMILES: *c1cccc(*)c1] 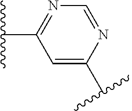 [SMILES: *c1cc(*)ncn1] | | H H |
| | 1c | 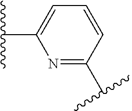 [SMILES: *c1cccc(*)n1] | | H |
| | 1d | 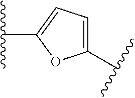 [SMILES: *c1ccc(*)o1] | | H |
| | 1e | 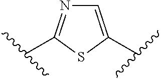 [SMILES: *c1cnc(*)s1] | 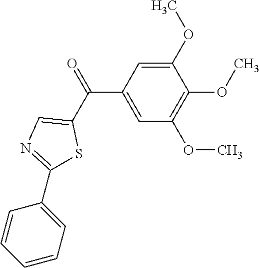 [SMILES: COc1cc(C(=O)c2cnc(-c3ccccc3)s2)cc(OC)c1OC] | H |
| | 1f | 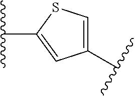 [SMILES: *c1csc(*)c1] | 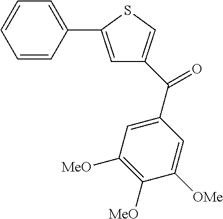 [SMILES: COc1cc(C(=O)c2csc(-c3ccccc3)c2)cc(OC)c1OC] | H |
| | 1g | 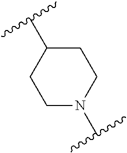 [SMILES: *C1CCN(*)CC1] | 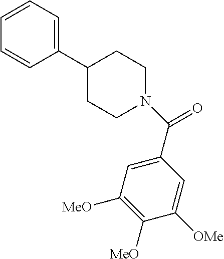 [SMILES: COc1cc(C(=O)N2CCC(c3ccccc3)CC2)cc(OC)c1OC] | H |
| | 1h | 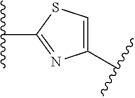 [SMILES: *c1csc(*)n1] | | H |
| | 1i | 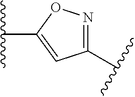 [SMILES: *c1cc(*)on1] | | H |
| | 1k | 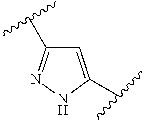 [SMILES: *c1cc(*)[nH]n1] | 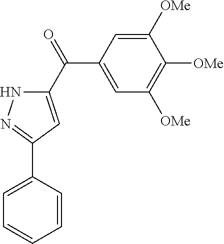 [SMILES: COc1cc(C(=O)c2cc(-c3ccccc3)n[nH]2)cc(OC)c1OC] | H |
| | 1l | 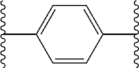 [SMILES: *c1ccc(*)cc1] | 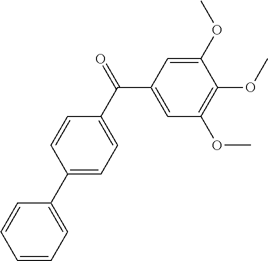 [SMILES: COc1cc(C(=O)c2ccc(-c3ccccc3)cc2)cc(OC)c1OC] | H |
| | 35a | 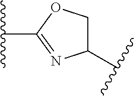 [SMILES: *C1=NC(*)CO1] | 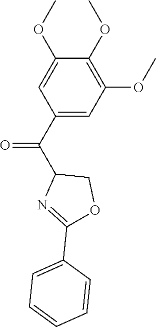 [SMILES: COc1cc(C(=O)C2COC(c3ccccc3)=N2)cc(OC)c1OC] | H |
| | 36a | 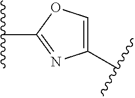 [SMILES: *c1coc(*)n1] | 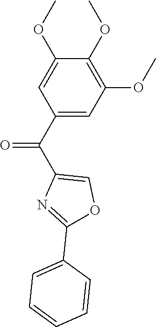 [SMILES: COc1cc(C(=O)c2coc(-c3ccccc3)n2)cc(OC)c1OC] | H |

The invention also encompasses methods of treating pancreatic cancer by administering at least one compound of formula (VI) in a therapeutically effective amount to a subject in need thereof, wherein the compound of Formula (VI) has the formula:

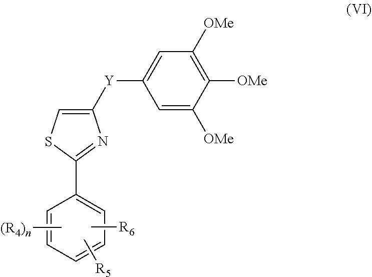

wherein
R₄, R₅ and R₆ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂; and Y is a bond or C═O, —C═S, —C═N—NH₂, —C═N—OH, —CH—OH, —C═CH—CN, —C═N—CN, —CH═CH—, C═C(CH₃)₂, —C═N—OMe, —(C═O)—NH, —NH—(C═O), —(C═O)—O, —O—(C═O), —(CH₂)₁₋₅—(C═O), (C═O)—(CH₂)₁₋₅, —(SO₂)—NH—, —NH—(SO₂)—, SO₂, SO or S;
n is an integer between 1-3; and
i is an integer from 1-5;
or its pharmaceutically acceptable salt, hydrate, polymorph, metabolite, tautomer or isomer.

The invention encompasses methods with the following compounds:

| | | | R₄, R₅ |
|---|---|---|---|
| | Compound | Y | and R₆ |
| 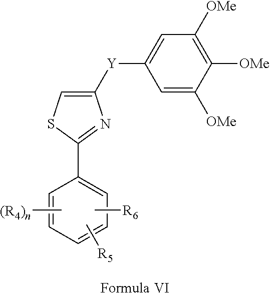 | 1h 2a 2b 2c 2d 2e 2f 2g 2h 2i 2j 2k | —C═O —C═C(CH₃)₂ —CH—OH —C═CH—CN (cis and trans) —C═N—NH₂ (cis and trans) —C═N—OH (cis and trans) —C═N—OMe (cis and trans) —(C═O)—NH— —NH—(C═O)— nothing —C═N—CN (cis and trans) C═O | H H H H H H H H H H H R₄ = R₆ = H R₅ = p-F |
| | 2l | C═O | R₄ = R₆ = H |
| | | | R₅ = p-OH |
| | 2m | C═O | R₄ = R₆ = H |
| | | | R₅ = p-CH₃ |
| | 2n | C═O | R₄ = R₆ = H |
| | | | R₅ = p-CH₂—CN |
| | 2o | C═O | R₄ = R₆ = H |
| | | | R₅ = p-N(CH₃)₂ |
| | 2p | C═O | R₄ = m-F; |
| | | | R₅ = p-F; |
| | | | R₆ = m-F; |
| | | | n = 1 |
| | 2q | C═O | R₄ = R₆ = H |
| | | | R₅ = |
| | | | p-CH₂(C═O)NH₂ |
| | 2r | C═O | R₄ = R₆ = H |
| | | | R₅ = p-CH₂NH₂ |
| | 2s | C═O | R₄ = R₆ = H |
| | | | R₅ = p-CH₂NH—CH₃ |
| | 2t | C═O | R₄ = m-OMe; |
| | | | R₅ = p-OMe; |
| | | | R₆ = m-OMe; |
| | | | n = 1 |
| | 2u | C═O | R₄ = R₆ = H |
| | | | R₅ = p-CH₂NMe₂ |

In one embodiment, this invention is directed to compound 3a:

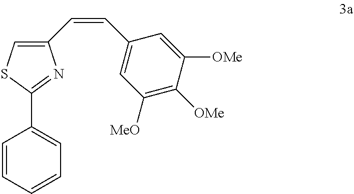 [SMILES: COc1cc(/C=C\c2csc(-c3ccccc3)n2)cc(OC)c1OC]

In one embodiment, this invention is directed to compound 3b:

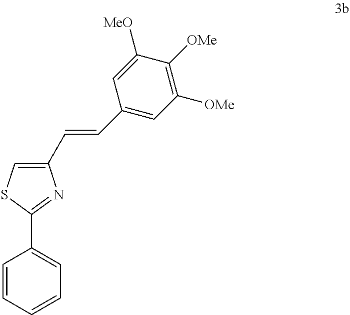 [SMILES: COc1cc(/C=C/c2csc(-c3ccccc3)n2)cc(OC)c1OC]

In one embodiment, this invention is directed to a compound of formula (VII)

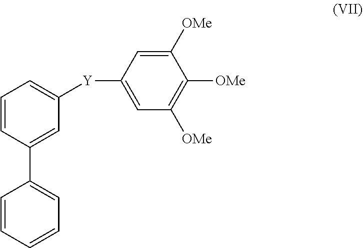

wherein
Y is a bond or C═O, —C═S, —C═N—NH₂, —C═N—OH, —CH—OH, —C═CH—CN, —C═N—CN, —CH═CH—, C═C(CH₃)₂, —C═N—OMe, —(C═O)—NH, —NH—(C═O), —(C═O)—O, —O—(C═O), —(CH₂)₁₋₅—(C═O), (C═O)—(CH₂)₁₋₅, —(SO₂)—NH—, —NH—(SO₂)—, SO₂, SO or S;
or its pharmaceutically acceptable salt, hydrate, polymorph, metabolite, tautomer or isomer.

In one embodiment, this invention is directed to the following compounds:

| | Compound | Y |
|---|---|---|
| 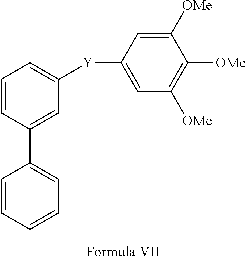 | 4a 4b 4c 4d | S SO₂ SO —(SO₂)—NH— |

In one embodiment, this invention is directed to a compound of formula (VIII)

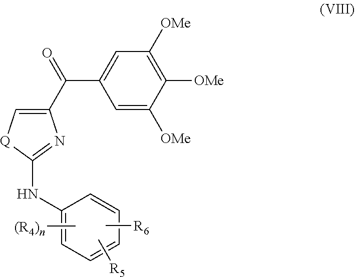

wherein
R₄, R₅ and R₆ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;

### Q is S, O or NH;

i is an integer between 0-5; and
n is an integer between 1-3;
or its pharmaceutically acceptable salt, hydrate, polymorph, metabolite, tautomer or isomer.

In one embodiment, this invention is directed to methods using the following compounds:

| | Compound | R₄ | R₅ | R₆ | Q |
|---|---|---|---|---|---|
| 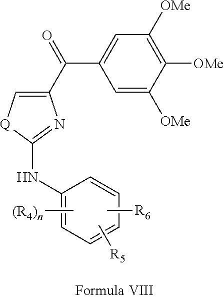 | 5a 5b 5c 5d 5e | H n = 1 H n = 1 H n = 1 H n = 1 H n = 1 | H p-CH₃ p-F p-Cl H | H H H H H | S S S S N |

The invention also encompasses methods of treating pancreatic cancer by administering at least one compound of formula (IX) in a therapeutically effective amount to a subject in need thereof, wherein the compound of Formula (IX):

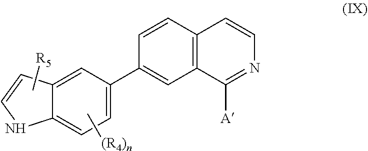

wherein
R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO— alkyl, COOH, —C(O)Ph, C(O)O-alkyl, C(O)H, —(O)NH₂ or NO₂;
A′ is halogen; substituted or unsubstituted single-, fused- or multiple-ring, aryl or (hetero)cyclic ring systems; substituted or unsubstituted, saturated or unsaturated N-heterocycles; substituted or unsubstituted, saturated or unsaturated S-heterocycles; substituted or unsubstituted, saturated or unsaturated O-heterocycles; substituted or unsubstituted, saturated or unsaturated cyclic hydrocarbons; or substituted or unsubstituted, saturated or unsaturated mixed heterocycles; wherein said A′ ring is optionally substituted by 1-5 substituents which are independently O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer between 1-5; and
n is an integer between 1-3;
or its pharmaceutically acceptable salt, hydrate, polymorph, metabolite, tautomer or isomer.

In one embodiment, a compound of Formula IX is represented by the structures of the following compounds:

| | Compound | A′ | R₄, R₅ |
|---|---|---|---|
| 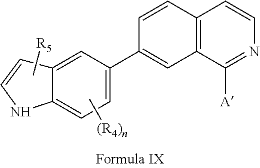 | 6a | 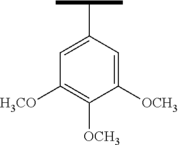 | H |
| | 6b | 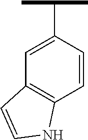 | H |
| | 6c | 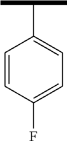 | H |
| | 6d | Cl | H |

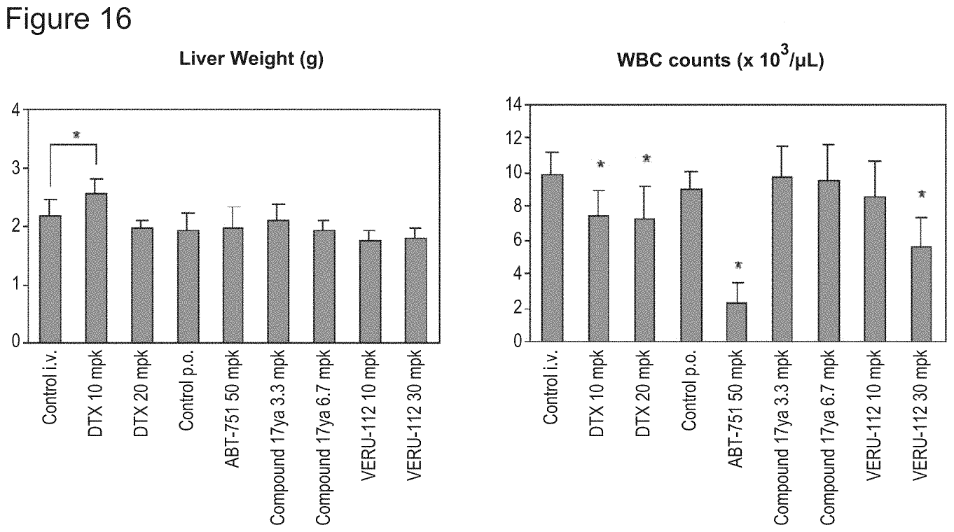

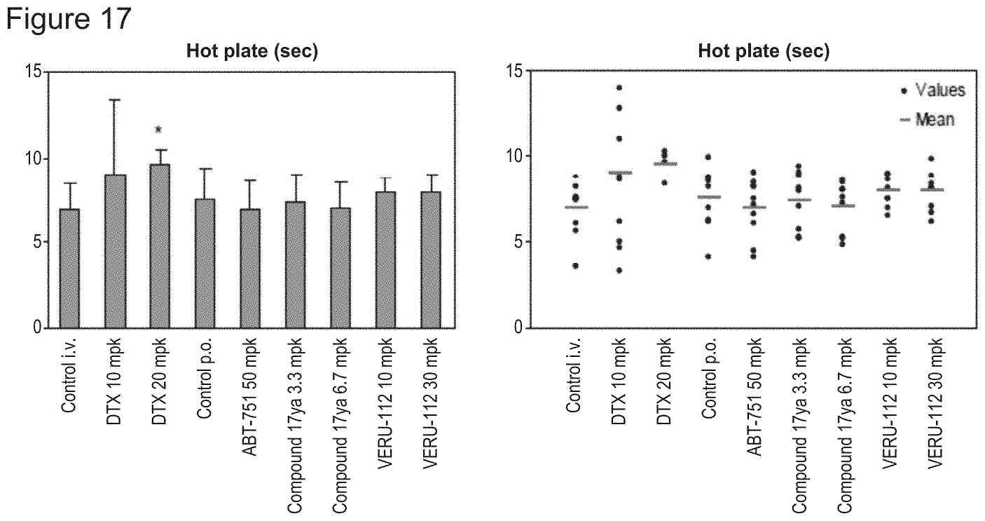

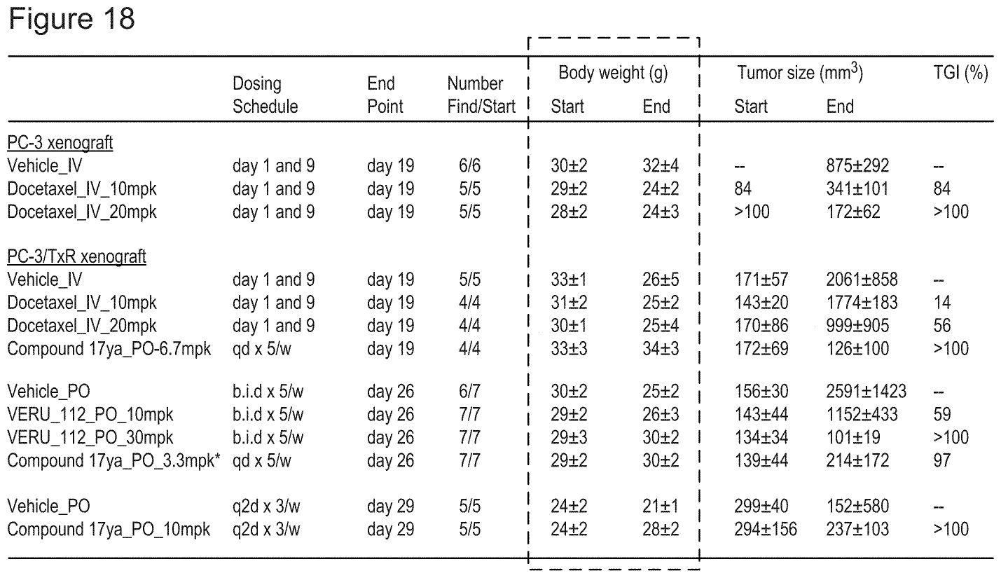

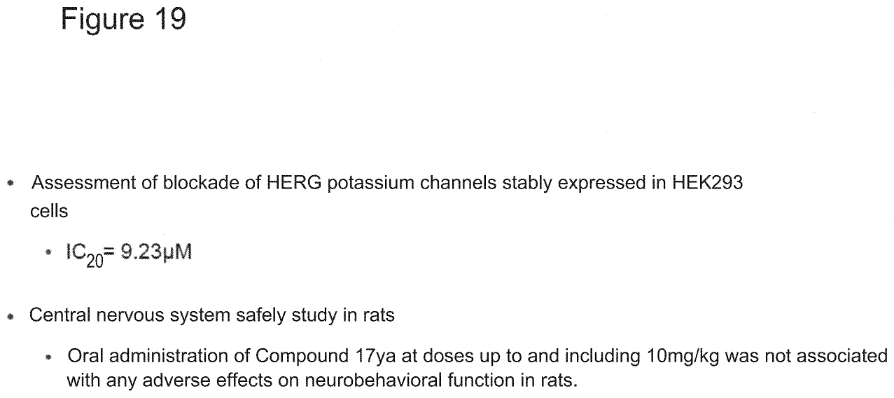

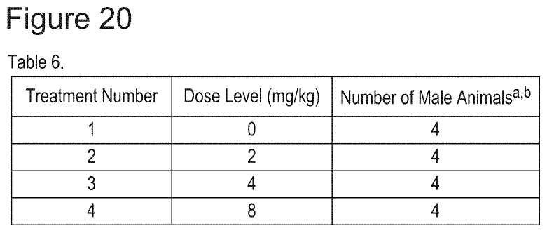

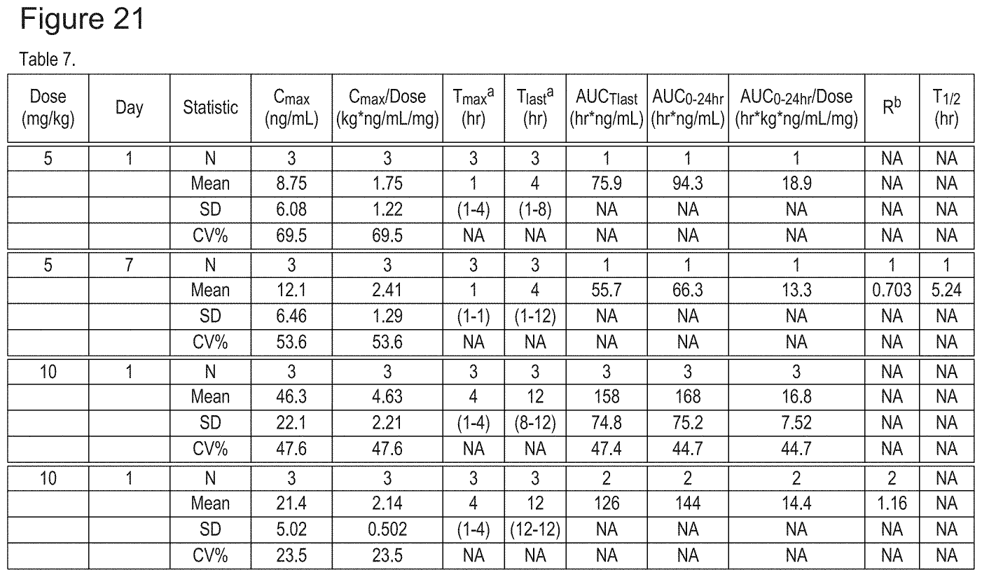

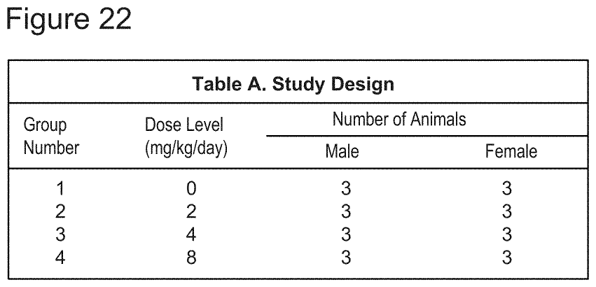

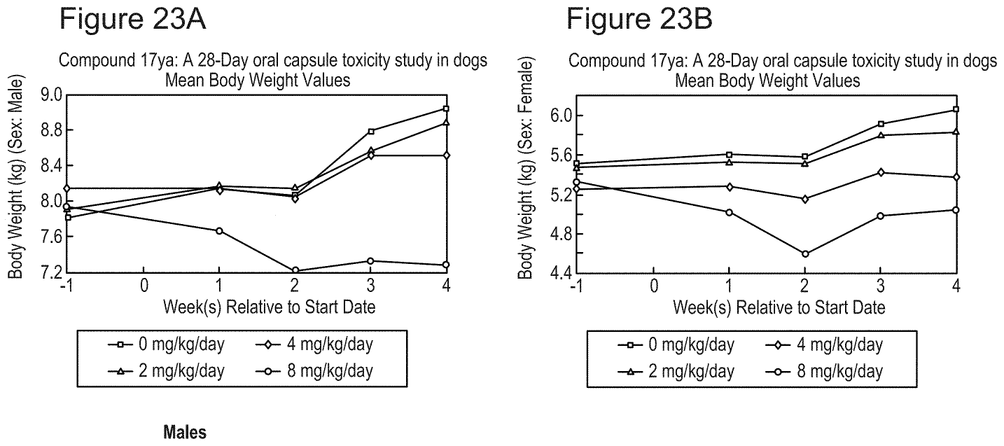

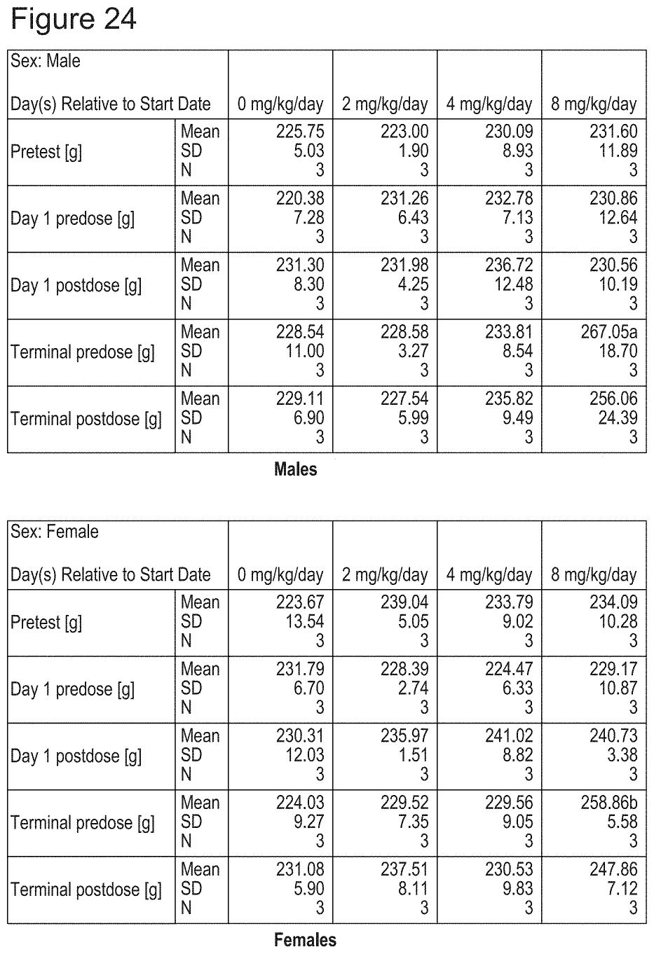

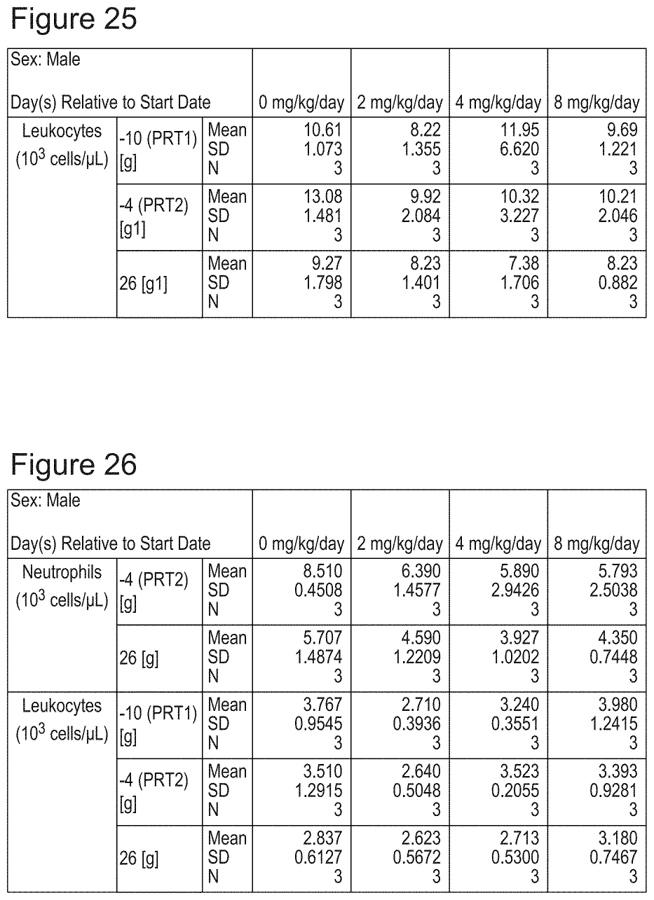

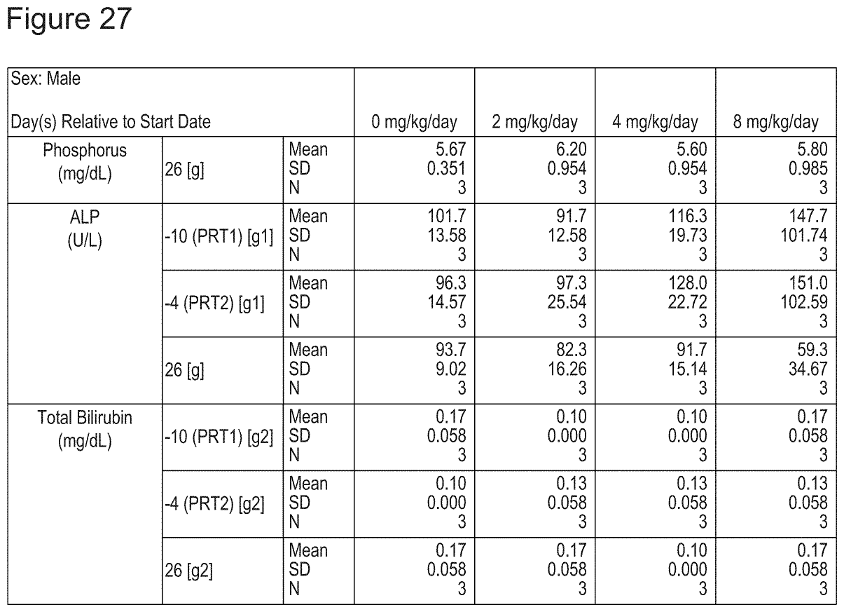

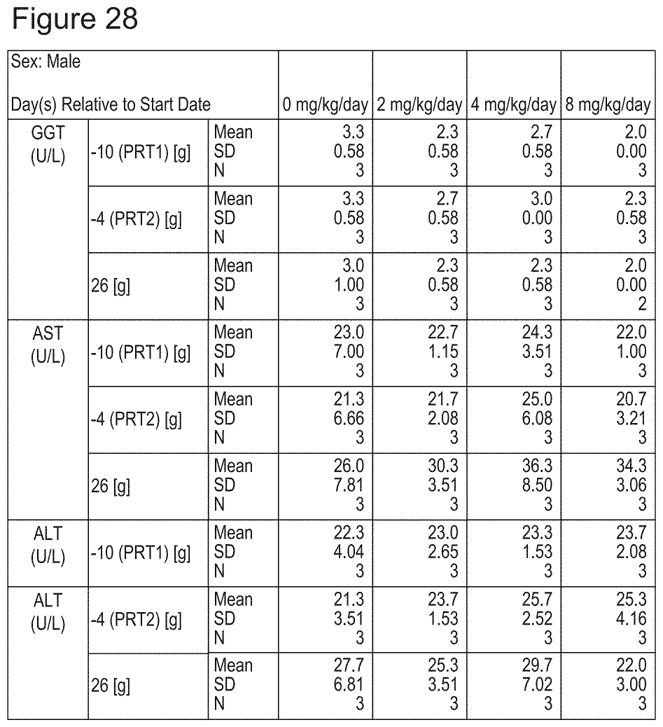

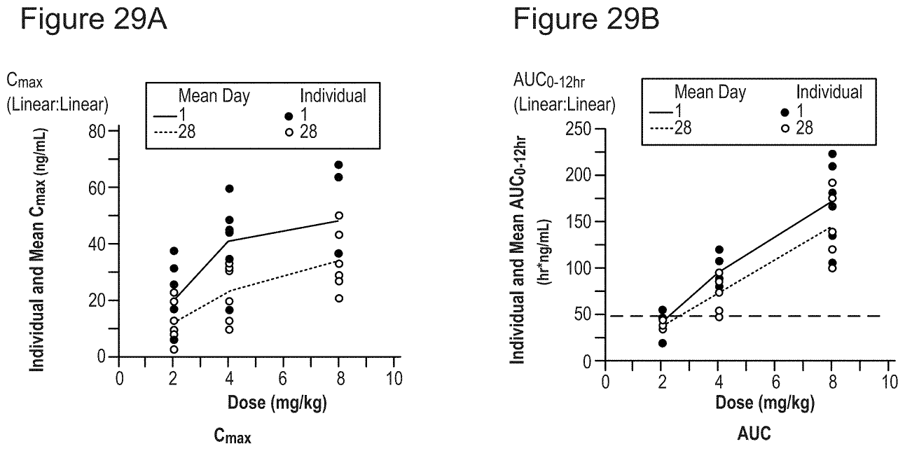

In another embodiment A′ of formula IX is a halogen. In one embodiment A′ of formula IX is a phenyl. In another embodiment A′ of formula IX is substituted phenyl. In another embodiment the substitution of A′ is halogen. In another embodiment the substitution is 4-F. In another embodiment the substitution is 3,4,5-(OCH₃)₃. In another embodiment, A′ of formula IX is substituted or unsubstituted 5-indolyl. In another embodiment, A′ of formula IX is substituted or unsubstituted 2-indolyl. In another embodiment, A′ of formula IX is substituted or unsubstituted 3-indolyl. In another embodiment, compounds of formula IX are presented in FIG. 16A.

The invention also encompasses methods of treating pancreatic cancer by administering at least one compound of formula (IXa) in a therapeutically effective amount to a subject in need thereof, wherein the compound of Formula (IXa):

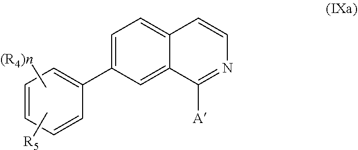

wherein
R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO— alkyl, COOH, —C(O)Ph, C(O)O-alkyl, C(O)H, —(O)NH₂ or NO₂;
A′ is halogen; substituted or unsubstituted single-, fused- or multiple-ring, aryl or (hetero)cyclic ring systems; substituted or unsubstituted, saturated or unsaturated N-heterocycles; substituted or unsubstituted, saturated or unsaturated S-heterocycles; substituted or unsubstituted, saturated or unsaturated O-heterocycles; substituted or unsubstituted, saturated or unsaturated cyclic hydrocarbons; or substituted or unsubstituted, saturated or unsaturated mixed heterocycles; wherein said A′ ring is optionally substituted by 1-5 substituents which are independently O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer between 1-5; and
n is an integer between 1-3;
or its pharmaceutically acceptable salt, hydrate, polymorph, metabolite, tautomer or isomer.

In another embodiment A′ of formula IXa is a halogen. In one embodiment A′ of formula IXa is a phenyl. In another embodiment A′ of formula IXa is substituted phenyl. In another embodiment the substitution of A′ is halogen. In another embodiment the substitution is 4-F. In another embodiment the substitution is 3,4,5-(OCH₃)₃. In another embodiment, A′ of formula IXa is substituted or unsubstituted 5-indolyl. In another embodiment, A′ of formula IXa is substituted or unsubstituted 2-indolyl. In another embodiment, A′ of formula IXa is substituted or unsubstituted 3-indolyl.

In another embodiment, a compound of formula IXa is 1-chloro-7-(4-fluorophenyl)isoquinoline. In another embodiment, a compound of formula IXa is 7-(4-fluorophenyl)-1-(1H-indol-5-yl)isoquinoline. In another embodiment, a compound of formula IXa is 7-(4-fluorophenyl)-1-(3,4,5-trimethoxyphenyl)isoquinoline. In another embodiment, a compound of formula IXa is 1,7-bis(4-fluorophenyl)isoquinoline (40). In another embodiment, a compound of formula IXa is 1,7-bis(3,4,5-trimethoxyphenyl)isoquinoline. In another embodiment, a compound of formula IXa is 1-(4-fluorophenyl)-7-(3,4,5-trimethoxyphenyl)isoquinoline. In another embodiment, a compound of formula IXa is 1-(1H-indol-5-yl)-7-(3,4,5-trimethoxyphenyl)isoquinoline. In another embodiment, a compound of formula IXa is 1-chloro-7-(3,4,5-trimethoxyphenyl)isoquinoline.

The invention also encompasses methods of treating pancreatic cancer by administering at least one compound of formula (XI) in a therapeutically effective amount to a subject in need thereof, wherein the compound of Formula (XI) is represented by the structure:

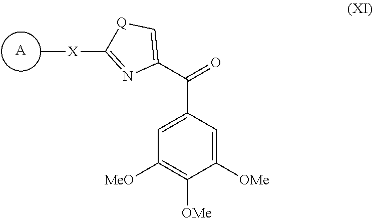

wherein
X is a bond, NH or S;

### Q is O, NH or S; and

A is substituted or unsubstituted single-, fused- or multiple-ring aryl or (hetero)cyclic ring systems; substituted or unsubstituted, saturated or unsaturated N-heterocycles; substituted or unsubstituted, saturated or unsaturated S-heterocycles; substituted or unsubstituted, saturated or unsaturated O-heterocycles; substituted or unsubstituted, saturated or unsaturated cyclic hydrocarbons; or substituted or unsubstituted, saturated or unsaturated mixed heterocycles; wherein said A ring is optionally substituted by 1-5 1-5 substituents which are independently O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂; and
i is an integer from 0-5;
or its pharmaceutically acceptable salt, hydrate, polymorph, metabolite, tautomer or isomer.

In one embodiment if Q of Formula XI is 5, then X is not a bond.

In one embodiment, A of compound of Formula XI is Ph. In another embodiment, A of compound of Formula XI is substituted Ph. In another embodiment, the substitution is 4-F. In another embodiment, the substitution is 4-Me. In another embodiment, Q of compound of Formula XI is S. In another embodiment, X of compound of Formula XI is NH. Non limiting examples of compounds of Formula XI are selected from: (2-(phenylamino)thiazol-4-yl)(3,4,5-trimethoxyphenyl)methanone (5a), (2-(p-tolylamino)thiazol-4-yl)(3,4,5-trimethoxyphenyl)methanone (5b), (2-(p-fluorophenylamino)thiazol-4-yl)(3,4,5-trimethoxyphenyl)methanone (5c), (2-(4-chlorophenylamino)thiazol-4-yl)(3,4,5-trimethoxyphenyl)methanone (5d), (2-(phenylamino)-1H-imidazol-4-yl)(3,4,5-trimethoxyphenyl)methanone (5e), (2-(phenylamino)thiazol-4-yl)(3,4,5-trimethoxyphenyl)methanone hydrochloride salt (5Ha), (2-(p-tolylamino)thiazol-4-yl)(3,4,5-trimethoxyphenyl)methanone hydrochloride salt (5Hb), (2-(p-fluorophenylamino)thiazol-4-yl)(3,4,5-trimethoxyphenyl)methanone hydrochloride salt (5Hc), (2-(4-chlorophenylamino)thiazol-4-yl)(3,4,5-trimethoxyphenyl)methanone hydrochloride salt (5Hd), (2-(phenylamino)-1H-imidazol-4-yl)(3,4,5-trimethoxyphenyl)methanone hydrochloride salt (5He).

The invention also encompasses methods of treating pancreatic cancer by administering at least one compound of formula XI(a) in a therapeutically effective amount to a subject in need thereof, wherein the compound of Formula XI(a) is represented by the structure:

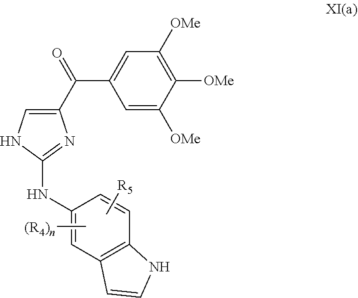

wherein R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4;
or its pharmaceutically acceptable salt, hydrate, polymorph, metabolite, tautomer or isomer.

The invention also encompasses methods of treating pancreatic cancer by administering at least one compound of formula XI(b) in a therapeutically effective amount to a subject in need thereof, wherein the compound of Formula XI(b) is represented by the structure:

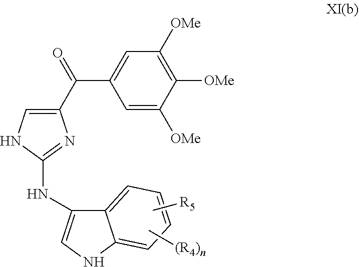

wherein R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4;
or its pharmaceutically acceptable salt, hydrate, polymorph, metabolite, tautomer or isomer.

The invention also encompasses methods of treating pancreatic cancer by administering at least one compound of formula XI(c) in a therapeutically effective amount to a subject in need thereof, wherein the compound of Formula XI(c) is represented by the structure: XI(c)

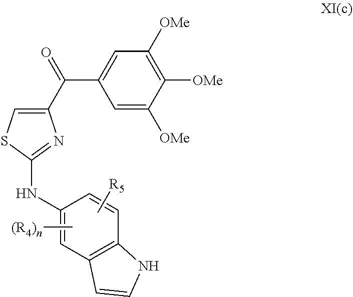

wherein R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4;
or its pharmaceutically acceptable salt, hydrate, polymorph, metabolite, tautomer or isomer.

The invention also encompasses methods of treating pancreatic cancer by administering at least one compound of formula XI(d) in a therapeutically effective amount to a subject in need thereof, wherein the compound of Formula XI(d) is represented by the structure: XI(d)

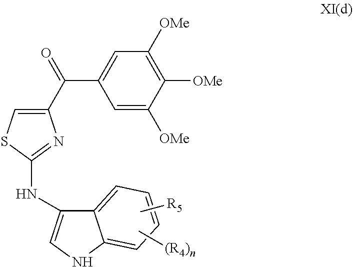

wherein R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4;
or its pharmaceutically acceptable salt, hydrate, polymorph, metabolite, tautomer or isomer.

The invention also encompasses methods of treating pancreatic cancer by administering at least one compound of formula XI(e) in a therapeutically effective amount to a subject in need thereof, wherein the compound of Formula XI(e) is represented by the structure:

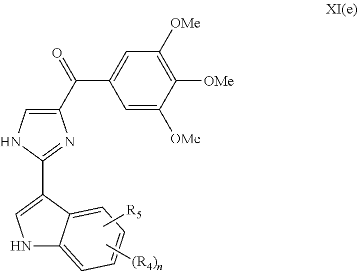

wherein R₄ and R₅ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4;
or its pharmaceutically acceptable salt, hydrate, polymorph, metabolite, tautomer or isomer.

The invention also encompasses methods of treating pancreatic cancer by administering compound 55 in a therapeutically effective amount to a subject in need thereof, wherein compound 55 is represented by the structure:

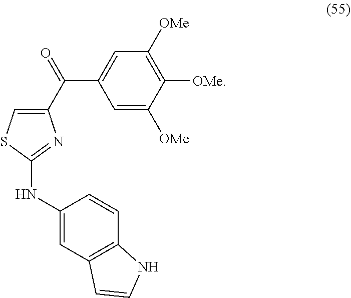 [SMILES: COc1cc(C(=O)c2csc(Nc3ccc4[nH]ccc4c3)n2)cc(OC)c1OC]

The invention also encompasses methods of treating pancreatic cancer by administering compound 17ya in a therapeutically effective amount to a subject in need thereof, wherein compound 17ya is represented by the structure:

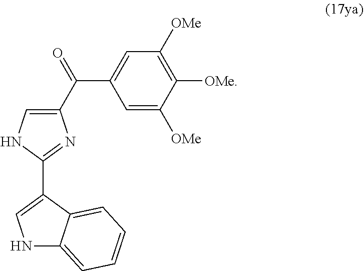 [SMILES: COc1cc(C(=O)c2c[nH]c(-c3c[nH]c4ccccc34)n2)cc(OC)c1OC]

The invention also encompasses methods of treating pancreatic cancer by administering at least one compound of the following structures in a therapeutically effective amount to a subject in need thereof, wherein the compound is selected from the following structures:

| compound | structure |
|---|---|
| 8 | 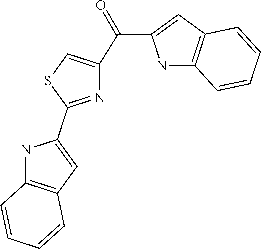 [SMILES: O=C(c1csc(-c2cc3ccccc3[nH]2)n1)c1cc2ccccc2[nH]1] |
| 9 | 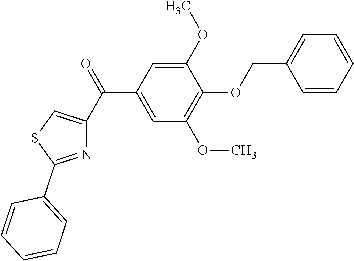 [SMILES: COc1cc(C(=O)c2csc(-c3ccccc3)n2)cc(OC)c1OCc1ccccc1] |
| 10 | 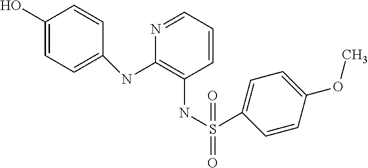 [SMILES: COc1ccc(S(=O)(=O)Nc2cccnc2Nc2ccc(O)cc2)cc1] |
| 11 | 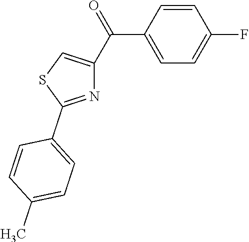 [SMILES: Cc1ccc(-c2nc(C(=O)c3ccc(F)cc3)cs2)cc1] |
| 12 | 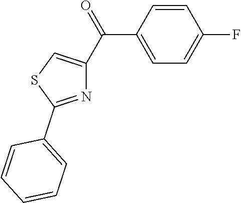 [SMILES: O=C(c1ccc(F)cc1)c1csc(-c2ccccc2)n1] |
| 13 | 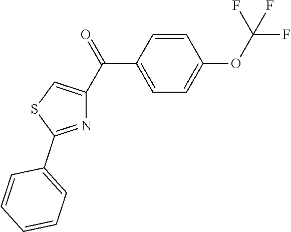 [SMILES: O=C(c1ccc(OC(F)(F)F)cc1)c1csc(-c2ccccc2)n1] |
| 14 | 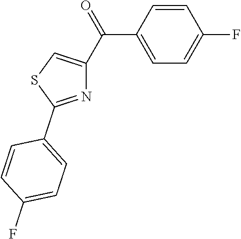 [SMILES: O=C(c1ccc(F)cc1)c1csc(-c2ccc(F)cc2)n1] |
| 16 | 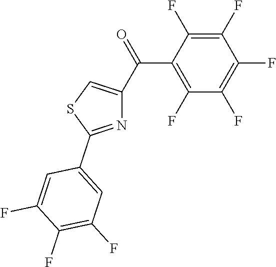 [SMILES: O=C(c1csc(-c2cc(F)c(F)c(F)c2)n1)c1c(F)c(F)c(F)c(F)c1F] |
| 17 | 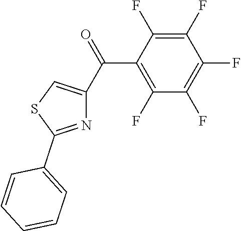 [SMILES: O=C(c1csc(-c2ccccc2)n1)c1c(F)c(F)c(F)c(F)c1F] |
| 18 | 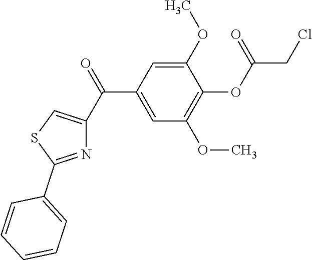 [SMILES: COc1cc(C(=O)c2csc(-c3ccccc3)n2)cc(OC)c1OC(=O)CCl] |
| 19 | 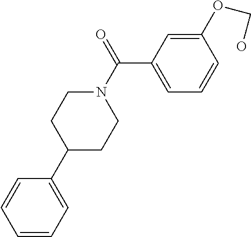 [SMILES: O=C(c1cccc(OCO)c1)N1CCC(c2ccccc2)CC1] |
| 20 | 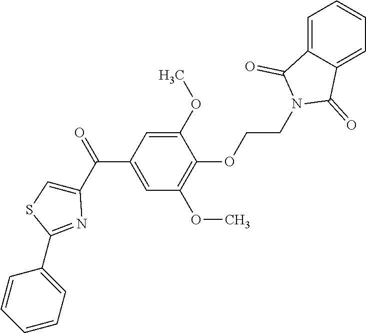 [SMILES: COc1cc(C(=O)c2csc(-c3ccccc3)n2)cc(OC)c1OCCN1C(=O)c2ccccc2C1=O] |
| 21 | 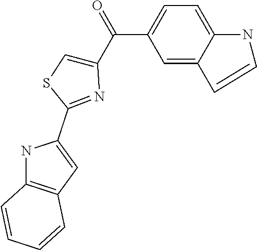 [SMILES: O=C(c1ccc2[nH]ccc2c1)c1csc(-c2cc3ccccc3[nH]2)n1] |
| 22 | 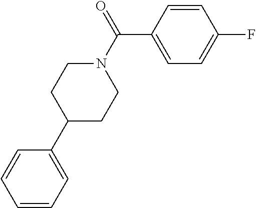 [SMILES: O=C(c1ccc(F)cc1)N1CCC(c2ccccc2)CC1] |
| 23 | 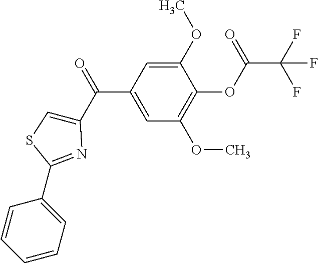 [SMILES: COc1cc(C(=O)c2csc(-c3ccccc3)n2)cc(OC)c1OC(=O)C(F)(F)F] |
| 24 | 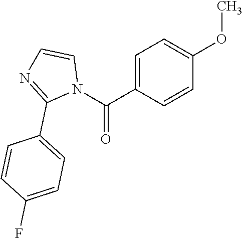 [SMILES: COc1ccc(C(=O)n2ccnc2-c2ccc(F)cc2)cc1] |
| 25 | 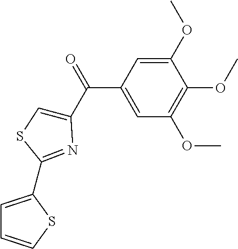 [SMILES: COc1cc(C(=O)c2csc(-c3cccs3)n2)cc(OC)c1OC] |
| 26 | 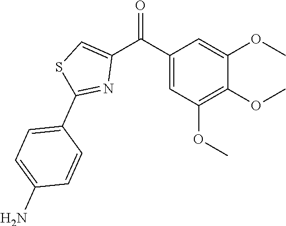 [SMILES: COc1cc(C(=O)c2csc(-c3ccc(N)cc3)n2)cc(OC)c1OC] |
| 27 | 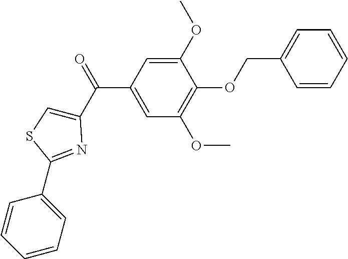 [SMILES: COc1cc(C(=O)c2csc(-c3ccccc3)n2)cc(OC)c1OCc1ccccc1] |
| 28 | 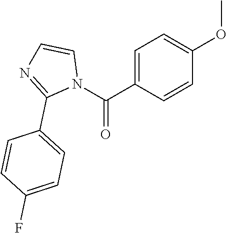 [SMILES: COc1ccc(C(=O)n2ccnc2-c2ccc(F)cc2)cc1] |
| 29 | 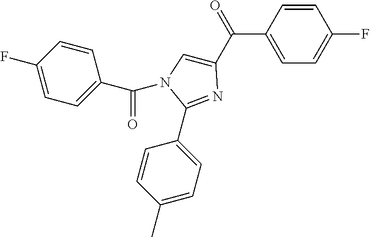 [SMILES: Cc1ccc(-c2nc(C(=O)c3ccc(F)cc3)cn2C(=O)c2ccc(F)cc2)cc1] |
| 30 | 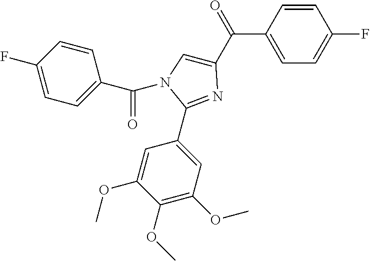 [SMILES: COc1cc(-c2nc(C(=O)c3ccc(F)cc3)cn2C(=O)c2ccc(F)cc2)cc(OC)c1OC] |
| 32 | 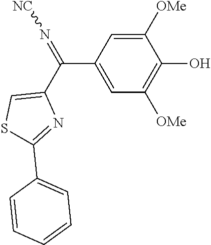 [SMILES: COc1cc(C(=NC#N)c2csc(-c3ccccc3)n2)cc(OC)c1O] |
| 33 | 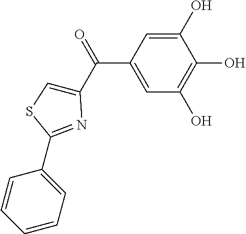 [SMILES: O=C(c1cc(O)c(O)c(O)c1)c1csc(-c2ccccc2)n1] |
| 34 | 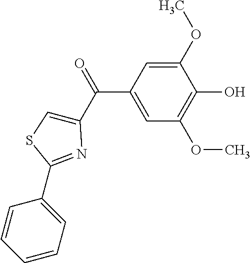 [SMILES: COc1cc(C(=O)c2csc(-c3ccccc3)n2)cc(OC)c1O] |
| 35 | 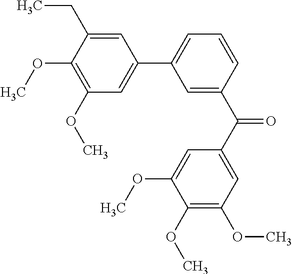 |
| 40 | 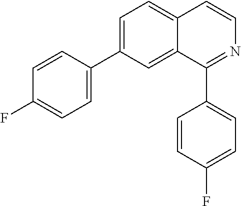 [SMILES: Fc1ccc(-c2ccc3ccnc(-c4ccc(F)cc4)c3c2)cc1] |
| 41 | 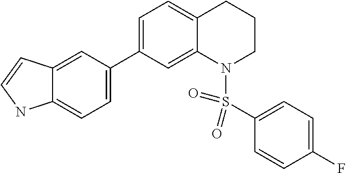 [SMILES: O=S(=O)(c1ccc(F)cc1)N1CCCc2ccc(-c3ccc4[nH]ccc4c3)cc21] |
| 42 | 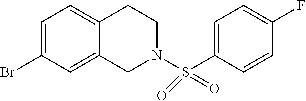 [SMILES: O=S(=O)(c1ccc(F)cc1)N1CCc2ccc(Br)cc2C1] |
| 43 | 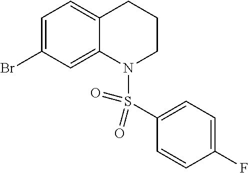 [SMILES: O=S(=O)(c1ccc(F)cc1)N1CCCc2ccc(Br)cc21] |
| 44 | 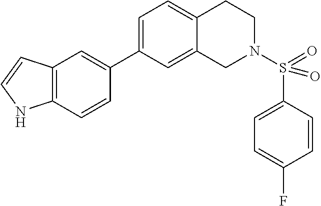 [SMILES: O=S(=O)(c1ccc(F)cc1)N1CCc2ccc(-c3ccc4[nH]ccc4c3)cc2C1] |
| 45 | 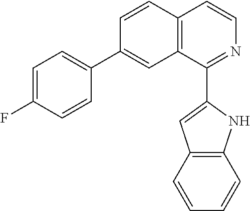 [SMILES: Fc1ccc(-c2ccc3ccnc(-c4cc5ccccc5[nH]4)c3c2)cc1] |
| 46 | 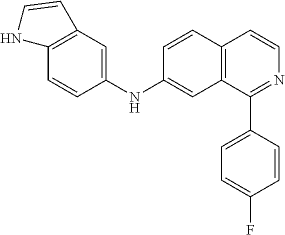 [SMILES: Fc1ccc(-c2nccc3ccc(Nc4ccc5[nH]ccc5c4)cc23)cc1] |
| 47 | 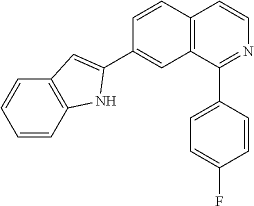 [SMILES: Fc1ccc(-c2nccc3ccc(-c4cc5ccccc5[nH]4)cc23)cc1] |
| 48 | 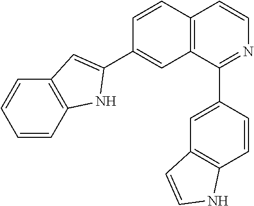 [SMILES: c1ccc2[nH]c(-c3ccc4ccnc(-c5ccc6[nH]ccc6c5)c4c3)cc2c1] |
| 49 | 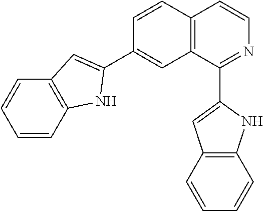 [SMILES: c1ccc2[nH]c(-c3ccc4ccnc(-c5cc6ccccc6[nH]5)c4c3)cc2c1] |
| 50 | 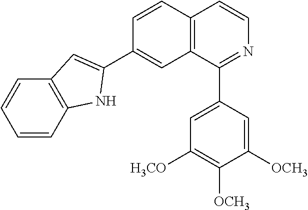 [SMILES: COc1cc(-c2nccc3ccc(-c4cc5ccccc5[nH]4)cc23)cc(OC)c1OC] |
| 51 | 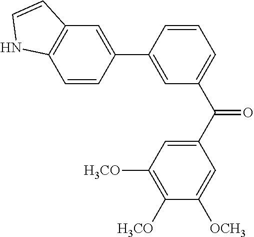 [SMILES: COc1cc(C(=O)c2cccc(-c3ccc4[nH]ccc4c3)c2)cc(OC)c1OC] |
| 52 | 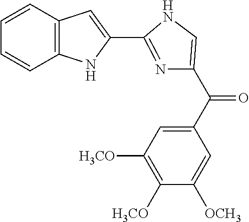 [SMILES: COc1cc(C(=O)c2c[nH]c(-c3cc4ccccc4[nH]3)n2)cc(OC)c1OC] |
| 53 | 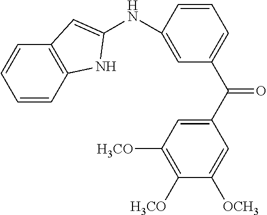 [SMILES: COc1cc(C(=O)c2cccc(Nc3cc4ccccc4[nH]3)c2)cc(OC)c1OC] |
| 54 | 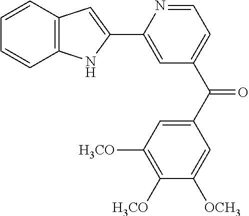 [SMILES: COc1cc(C(=O)c2ccnc(-c3cc4ccccc4[nH]3)c2)cc(OC)c1OC] |

It is well understood that in structures presented in this invention wherein the nitrogen atom has less than 3 bonds, H atoms are present to complete the valence of the nitrogen.

In one embodiment the A, A′ and/or C groups of formula I, I(a), IV, IX, IX(a) and XI are independently substituted and unsubstituted furanyl, indolyl, pyridinyl, phenyl, biphenyl, triphenyl, diphenylmethane, adamantane-yl, fluorene-yl, and other heterocyclic analogs such as, e.g., pyrrolyl, pyrazolyl, imidazolyl, pyridinyl, pyrimidinyl, pyrazinyl, pyridazinyl, triazinyl, tetrazinyl, pyrrolizinyl, indolyl, isoquinolinyl, quinolinyl, isoquinolinyl, benzimidazolyl, indazolyl, quinolizinyl, cinnolinyl, quinalolinyl, phthalazinyl, naphthyridinyl, quinoxalinyl, oxiranyl, oxetanyl, tetrahydrofuranyl, tetrahydropyranyl, dioxanyl, furanyl, pyrylium, benzofuranyl, benzodioxolyl, thiranyl, thietanyl, tetrahydrothiophene-yl, dithiolanyl, tetrahydrothiopyranyl, thiophene-yl, thiepinyl, thianaphthenyl, oxathiolanyl, morpholinyl, thioxanyl, thiazolyl, isothiazolyl, thiadiazolyl, oxazolyl, isoxazolyl, oxadiaziolyl).

In one embodiment, the A, A′ and/or C groups is substituted and unsubstituted phenyl. In another embodiment, the A, A′ and/or C groups is phenyl substituted by Cl, F or methyl. In one embodiment, the A, A′ and/or C groups is substituted and unsubstituted isoquinolinyl. In one embodiment, the A, A′ and/or C groups include substituted and unsubstituted indolyl groups; most preferably, substituted and unsubstituted 3-indolyl and 5-indolyl.

In one embodiment, the A, A′ and/or C groups of formula I, I(a), IV, IX, IX(a) and XI can be substituted or unsubstituted. Thus, although the exemplary groups recited in the preceding paragraph are unsubstituted, it should be appreciated by those of skill in the art that these groups can be substituted by one or more, two or more, three or more, and even up to five substituents (other than hydrogen).

In one embodiment, the most preferred A, A′ and/or C groups are substituted by 3,4,5-trimethoxyphenyl. In another embodiment the A, A′ and/or C groups are substituted by alkoxy. In another embodiment the A, A′ and/or C groups are substituted by methoxy. In another embodiment the A, A′ and/or C groups are substituted by alkyl. In another embodiment the A, A′ and/or C groups are substituted by methyl. In another embodiment the A, A′ and/or C groups are substituted by halogen. In another embodiment, the A, A′ and/or C groups are substituted by F. In another embodiment, the A, A′ and/or C groups are substituted by Cl. In another embodiment, the A, A′ and/or C rings are substituted by Br.

The substituents of these A, A′ and/or C groups of formula I, I(a), IV, IX, IX(a) and XI are independently selected from the group of hydrogen (e.g., no substitution at a particular position), hydroxyl, an aliphatic straight- or branched-chain C₁ to C₁₀ hydrocarbon, alkoxy, haloalkoxy, aryloxy, nitro, cyano, alkyl-CN, halo (e.g., F, Cl, Br, I), haloalkyl, dihaloalkyl, trihaloalkyl, COOH, C(O)Ph, C(O)-alkyl, C(O)O-alkyl, C(O)H, C(O)NH₂, —OC(O)CF₃, OCH₂Ph, amino, aminoalkyl, alkylamino, mesylamino, dialkylamino, arylamino, amido, NHC(O)-alkyl, urea, alkyl-urea, alkylamido (e.g., acetamide), haloalkylamido, arylamido, aryl, and C₅ to C₇ cycloalkyl, arylalkyl, and combinations thereof. Single substituents can be present at the ortho, meta, or para positions. When two or more substituents are present, one of them is preferably, though not necessarily, at the poro position.

In one embodiment the B group of formula I, I(a), II, III, IV, IVa and V is selected from substituted or unsubstituted-thiazole, thiazolidine, oxazole, oxazoline, oxazolidine, benzene, pyrimidine, imidazole, pyridine, furan, thiophene, isoxazole, piperidine, pyrazole, indole and isoquinoline, wherein said B ring is linked via any two positions of the ring to X and Y or directly to the A and/or C rings.

In one embodiment the B group of formula I, I(a), II, III, IV, IVa and V is unsubstituted. In another embodiment the B group of formula I, I(a), II, III, IV, IVa and V is:

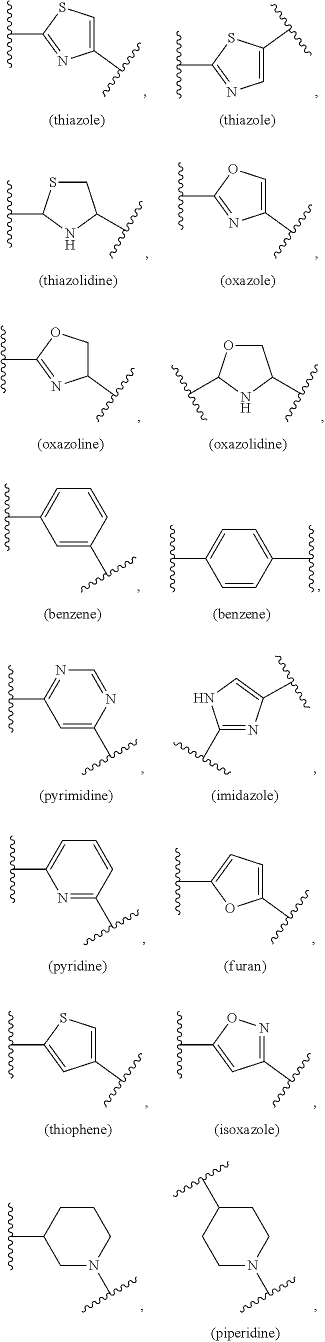

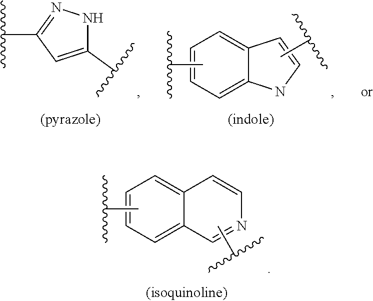

In another embodiment the B group of formula I, I(a), II, III, IV, Ia and V is substituted. In another embodiment the B group of formula I, I(a), II, III, IV, IVa and V is:

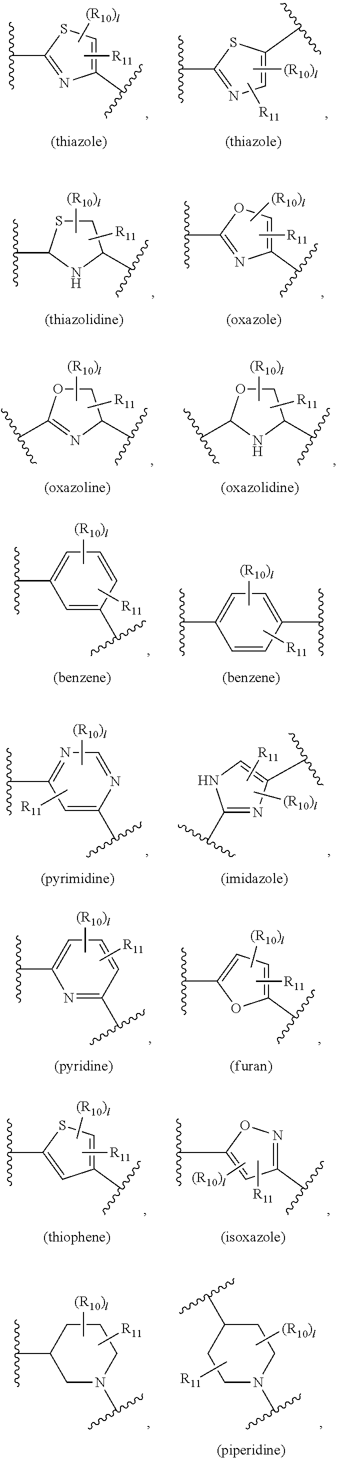

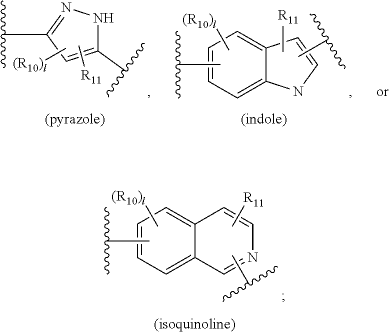

wherein R₁₀ and R₁₁ are independently hydrogen, O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, haloalkyl, alkylamino, aminoalkyl, —OCH₂Ph, —NHCO-alkyl, COOH, —C(O)Ph, C(O)O-alkyl, C(O)H, —C(O)NH₂ or NO₂.

In another embodiment the B group is

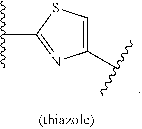 [SMILES: *c1csc(*)n1]

In another embodiment the B group is

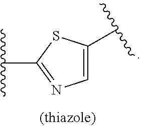 [SMILES: *c1cnc(*)s1]

In another embodiment the B group is

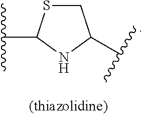 [SMILES: *C1CSC(*)N1]

In another embodiment the B group is

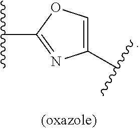 [SMILES: *c1coc(*)n1]

In another embodiment the B group is

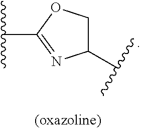 [SMILES: *C1=NC(*)CO1]

In another embodiment the B group is

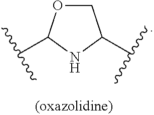 [SMILES: *C1COC(*)N1]

In another embodiment the B group is

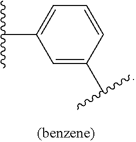 [SMILES: *c1cccc(*)c1]

In another embodiment the B group is

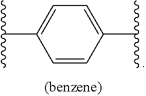 [SMILES: *c1ccc(*)cc1]

In another embodiment the B group is

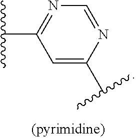 [SMILES: *c1cc(*)ncn1]

In another embodiment the B group is

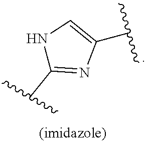 [SMILES: *c1c[nH]c(*)n1]

In another embodiment the B group is

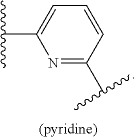 [SMILES: *c1cccc(*)n1]

In another embodiment the B group is

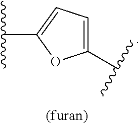 [SMILES: *c1ccc(*)o1]

In another embodiment the B group is

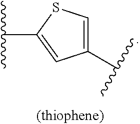 [SMILES: *c1csc(*)c1]

In another embodiment the B group is

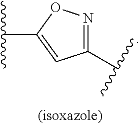 [SMILES: *c1cc(*)on1]

In another embodiment the B group is

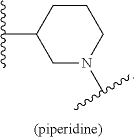 [SMILES: *C1CCCN(*)C1]

In another embodiment the B group is

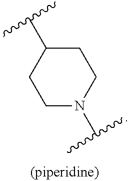 [SMILES: *C1CCN(*)CC1]

In another embodiment the B group is

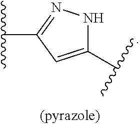 [SMILES: *c1cc(*)[nH]n1]

In another embodiment the B group is

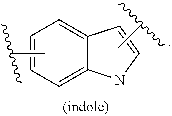

In another embodiment the B group is

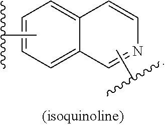

In one embodiment the B group of formula I, I(a), II, III, IV, IVa and V is substituted by R₁₀ and R₁₁. In another embodiment, R₁₀ and R₁₁ are both hydrogens. In another embodiment, R₁₀ and R₁₁ are independently O-alkyl. In another embodiment, R₁₀ and R₁₁ are independently O-haloalkyl. In another embodiment, R₁₀ and R₁₁ are independently F. In another embodiment, R₁₀ and R₁₁ are independently Cl. In another embodiment, R₁₀ and R₁₁ are independently Br. In another embodiment, R₁₀ and R₁₁ are independently I. In another embodiment, R₁₀ and R₁₁ are independently haloalkyl. In another embodiment, R₁₀ and R₁₁ are independently CF₃. In another embodiment, R₁₀ and R₁₁ are independently CN. In another embodiment, R₁₀ and R₁₁ are independently —CH₂CN. In another embodiment, R₁₀ and R₁₁ are independently NH₂. In another embodiment, R₁₀ and R₁₁ are independently hydroxyl. In another embodiment, R₁₀ and R₁₁ are independently —(CH₂)ᵢNHCH₃. In another embodiment, R₁₀ and R₁₁ are independently —(CH₂)ᵢNH₂. In another embodiment, R₁₀ and R₁₁ are independently —(CH₂)ᵢN(CH₃)₂. In another embodiment, R₁₀ and R₁₁ are independently —OC(O)CF₃. In another embodiment, R₁₀ and R₁₁ are independently C₁-C₅ linear or branched alkyl. In another embodiment, R₁₀ and R₁₁ are independently C₁-C₅ linear or branched haloalkyl. In another embodiment, R₁₀ and R₁₁ are independently C₁-C₅ linear or branched alkylamino. In another embodiment, R₁₀ and R₁₁ are independently C₁-C₅ linear or branched aminoalkyl. In another embodiment, R₁₀ and R₁₁ are independently —OCH₂Ph. In another embodiment, R₁₀ and R₁₁ are independently —NHCO-alkyl. In another embodiment, R₁₀ and R₁₁ are independently COOH. In another embodiment, R₁₀ and R₁₁ are independently —C(O)Ph. In another embodiment, R₁₀ and R₁₁ are independently C(O)O-alkyl. In another embodiment, R₁₀ and R₁₁ are independently C(O)H. In another embodiment, R₁₀ and R₁₁ are independently —C(O)NH₂. In another embodiment, R₁₀ and R₁₁ are independently NO₂.

In another embodiment the B group of formula I, I(a), II, III, IV, IVa and V is (thiazole),

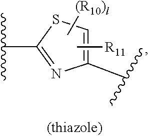

wherein R₁₀ and R₁₁ are independently H and I is 1. In another embodiment, R₁₀ and R₁₁ are independently O-alkyl. In another embodiment, R₁₀ and R₁₁ are independently O-haloalkyl. In another embodiment, R₁₀ and R₁₁ are independently F. In another embodiment, R₁₀ and R₁₁ are independently Cl. In another embodiment, R₁₀ and R₁₁ are independently Br. In another embodiment, R₁₀ and R₁₁ are independently I. In another embodiment, R₁₀ and R₁₁ are independently haloalkyl. In another embodiment, R₁₀ and R₁₁ are independently CF₃. In another embodiment, R₁₀ and R₁₁ are independently CN. In another embodiment, R₁₀ and R₁₁ are independently —CH₂CN. In another embodiment, R₁₀ and R₁₁ are independently NH₂. In another embodiment, R₁₀ and R₁₁ are independently hydroxyl. In another embodiment, R₁₀ and R₁₁ are independently —(CH₂)ᵢNHCH₃. In another embodiment, R₁₀ and R₁₁ are independently —(CH₂)ᵢNH₂. In another embodiment, R₁₀ and R₁₁ are independently —(CH₂)ᵢN(CH₃)₂. In another embodiment, R₁₀ and R₁₁ are independently —OC(O)CF₃. In another embodiment, R₁₀ and R₁₁ are independently C₁-C₅ linear or branched alkyl. In another embodiment, R₁₀ and R₁₁ are independently C₁-C₅ linear or branched haloalkyl. In another embodiment, R₁₀ and R₁₁ are independently C₁-C₅ linear or branched alkylamino. In another embodiment, R₁₀ and R₁₁ are independently C₁-C₅ linear or branched aminoalkyl. In another embodiment, R₁₀ and R₁₁ are independently —OCH₂Ph. In another embodiment, R₁₀ and R₁₁ are independently —NHCO-alkyl. In another embodiment, R₁₀ and R₁₁ are independently COOH. In another embodiment, R₁₀ and R₁₁ are independently —C(O)Ph. In another embodiment, R₁₀ and R₁₁ are independently C(O)O-alkyl. In another embodiment, R₁₀ and R₁₁ are independently C(O)H. In another embodiment, R₁₀ and R₁₁ are independently —C(O)NH₂. In another embodiment, R₁₀ and R₁₁ are independently NO₂.

In another embodiment the B group of formula I, I(a), II, III, IV, IVa and V is

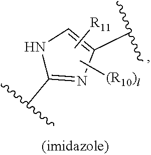

wherein R₁₀ and R₁₁ are independently H and I is 1. In another embodiment, R₁₀ and R₁₁ are independently O-alkyl. In another embodiment, R₁₀ and R₁₁ are independently O-haloalkyl. In another embodiment, R₁₀ and R₁₁ are independently F. In another embodiment, R₁₀ and R₁₁ are independently Cl. In another embodiment, R₁₀ and R₁₁ are independently Br. In another embodiment, R₁₀ and R₁₁ are independently I. In another embodiment, R₁₀ and R₁₁ are independently haloalkyl. In another embodiment, R₁₀ and R₁₁ are independently CF₃. In another embodiment, R₁₀ and R₁₁ are independently CN. In another embodiment, R₁₀ and R₁₁ are independently —CH₂CN. In another embodiment, R₁₀ and R₁₁ are independently NH₂. In another embodiment, R₁₀ and R₁₁ are independently hydroxyl. In another embodiment, R₁₀ and R₁₁ are independently —(CH₂)ᵢNHCH₃. In another embodiment, R₁₀ and R₁₁ are independently —(CH₂)ᵢNH₂. In another embodiment, R₁₀ and R₁₁ are independently —(CH₂)ᵢN(CH₃)₂. In another embodiment, R₁₀ and R₁₁ are independently —OC(O)CF₃. In another embodiment, R₁₀ and R₁₁ are independently C₁-C₅ linear or branched alkyl. In another embodiment, R₁₀ and R₁₁ are independently C₁-C₅ linear or branched haloalkyl. In another embodiment, R₁₀ and R₁₁ are independently C₁-C₅ linear or branched alkylamino. In another embodiment, R₁₀ and R₁₁ are independently C₁-C₅ linear or branched aminoalkyl. In another embodiment, R₁₀ and R₁₁ are independently —OCH₂Ph. In another embodiment, R₁₀ and R₁₁ are independently —NHCO-alkyl. In another embodiment, R₁₀ and R₁₁ are independently COOH. In another embodiment, R₁₀ and R₁₁ are independently —C(O)Ph. In another embodiment, R₁₀ and R₁₁ are independently C(O)O-alkyl. In another embodiment, R₁₀ and R₁₁ are independently C(O)H. In another embodiment, R₁₀ and R₁₁ are independently —C(O)NH₂. In another embodiment, R₁₀ and R₁₁ are independently NO₂.

In another embodiment the B group of formula I, I(a), II, III, IV, IVa and V is

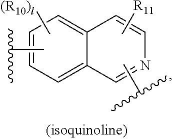

wherein R₁₀ and R₁₁ are independently H and I is 1. In another embodiment, R₁₀ and R₁₁ are independently O-alkyl. In another embodiment, R₁₀ and R₁₁ are independently O-haloalkyl. In another embodiment, R₁₀ and R₁₁ are independently F. In another embodiment, R₁₀ and R₁₁ are independently Cl. In another embodiment, R₁₀ and R₁₁ are independently Br. In another embodiment, R₁₀ and R₁₁ are independently I. In another embodiment, R₁₀ and R₁₁ are independently haloalkyl. In another embodiment, R₁₀ and R₁₁ are independently CF₃. In another embodiment, R₁₀ and R₁₁ are independently CN. In another embodiment, R₁₀ and R₁₁ are independently —CH₂CN. In another embodiment, R₁₀ and R₁₁ are independently NH₂. In another embodiment, R₁₀ and R₁₁ are independently hydroxyl. In another embodiment, R₁₀ and R₁₁ are independently —(CH₂)ᵢNHCH₃. In another embodiment, R₁₀ and R₁₁ are independently —(CH₂)ᵢNH₂. In another embodiment, R₁₀ and R₁₁ are independently —(CH₂)ᵢN(CH₃)₂. In another embodiment, R₁₀ and R₁₁ are independently —OC(O)CF₃. In another embodiment, R₁₀ and R₁₁ are independently C₁-C₅ linear or branched alkyl. In another embodiment, R₁₀ and R₁₁ are independently C₁-C₅ linear or branched haloalkyl. In another embodiment, R₁₀ and R₁₁ are independently C₁-C₅ linear or branched alkylamino. In another embodiment, R₁₀ and R₁₁ are independently C₁-C₅ linear or branched aminoalkyl. In another embodiment, R₁₀ and R₁₁ are independently —OCH₂Ph. In another embodiment, R₁₀ and R₁₁ are independently —NHCO-alkyl. In another embodiment, R₁₀ and R₁₁ are independently COOH. In another embodiment, R₁₀ and R₁₁ are independently —C(O)Ph. In another embodiment, R₁₀ and R₁₁ are independently C(O)O-alkyl. In another embodiment, R₁₀ and R₁₁ are independently C(O)H. In another embodiment, R₁₀ and R₁₁ are independently —C(O)NH₂. In another embodiment, R₁₀ and R₁₁ are independently NO₂.

In one embodiment, the X bridge of formula I, la, II, II, IV, Iva and XI is a bond. In another embodiment, the X bridge is NH. In another embodiment, the X bridge is C₁ to C₅ hydrocarbon. In another embodiment, the X bridge is CH₂. In another embodiment, the X bridge is —CH₂—CH₂—. In another embodiment, the X bridge is O. In another embodiment, the X bridge is S.

In one embodiment, the Y bridge of formula I, Ia, II, III, IV, IVa, VI, and VII is C═O. In another embodiment, the Y bridge is C═S. In another embodiment, the Y bridge is C═N(NH₂)—. In another embodiment, the Y bridge is —C═NOH. In another embodiment, the Y bridge is —CH—OH. In another embodiment, the Y bridge is —C═CH—(CN). In another embodiment, the Y bridge is —C═N(CN). In another embodiment, the Y bridge is —C═C(CH₃)₂. In another embodiment, the Y bridge is —C═N—OMe. In another embodiment, the Y bridge is —(C═O)NH—. In another embodiment, the Y bridge is —NH(C═O)—. In another embodiment, the Y bridge is —(C═O)—O. In another embodiment, the Y bridge is —O—(C═O). In another embodiment, the Y bridge is —(CH₂)₁₋₅—(C═O). In another embodiment, the Y bridge is —(C═O)—(CH₂)₁₋₅. In another embodiment, the Y bridge is S. In another embodiment, the Y bridge is SO. In another embodiment, the Y bridge is SO₂. In another embodiment, the Y bridge is —CH═CH—. In another embodiment, the Y bridge is —(SO₂)—NH—. In another embodiment, the Y bridge is —NH—(SO₂)—.

In one embodiment, R₁, R₂, R₃, R₄, R₅ and R₆ of formula Ia, II, III, IV, IV(a), V, VI, VIII, IX, IX(a), XI(a), XI(b), XI(c), XI(d) and XI(e) are independently hydrogen. In another embodiment, R₁, R₂, R₃, R₄, R₅ and R₆ are independently O-alkyl. In another embodiment, R₁, R₂, R₃, R₄, R₅ and R₆ are independently O-haloalkyl. In another embodiment, R₁, R₂, R₃, R₄, R₅ and R₆ are independently F. In another embodiment, R₁, R₂, R₃, R₄, R₅ and R₆ are independently Cl. In another embodiment, R₁, R₂, R₃, R₄, R₅ and R₆ are independently Br. In another embodiment, R₁, R₂, R₃, R₄, R₅ and R₆ are independently I. In another embodiment, R₁, R₂, R₃, R₄, R₅ and R₆ are independently haloalkyl. In another embodiment, R₁, R₂, R₃, R₄, R₅ and R₆ are independently CF₃. In another embodiment, R₁, R₂, R₃, R₄, R₅ and R₆ are independently CN. In another embodiment, R₁, R₂, R₃, R₄, R₅ and R₆ are independently —CH₂CN. In another embodiment, R₁, R₂, R₃, R₄, R₅ and R₆ are independently NH₂. In another embodiment, R₁, R₂, R₃, R₄, R₅ and R₆ are independently hydroxyl. In another embodiment, R₁, R₂, R₃, R₄, R₅ and R₆ are independently —(CH₂)ᵢNHCH₃. In another embodiment, R₁, R₂, R₃, R₄, R₅ and R₆ are independently —(CH₂)ᵢNH₂. In another embodiment, R₁, R₂, R₃, R₄, R₅ and R₆ are independently —(CH₂)ᵢN(CH₃)₂. In another embodiment, R₁, R₂, R₃, R₄, R₅ and R₆ are independently —OC(O)CF₃. In another embodiment, R₁, R₂, R₃, R₄, R₅ and R₆ are independently C₁-C₅ linear or branched alkyl. In another embodiment, R₁, R₂, R₃, R₄, R₅ and R₆ are independently haloalkyl. In another embodiment, R₁, R₂, R₃, R₄, R₅ and R₆ are independently alkylamino. In another embodiment, R₁, R₂, R₃, R₄, R₅ and R₆ are independently aminoalkyl. In another embodiment, R₁, R₂, R₃, R₄, R₅ and R₆ are independently —OCH₂Ph. In another embodiment, R₁, R₂, R₃, R₄, R₅ and R₆ are independently —NHCO-alkyl. In another embodiment, R₁, R₂, R₃, R₄, R₅ and R₆ are independently COOH. In another embodiment, R₁, R₂, R₃, R₄, R₅ and R₆ are independently —C(O)Ph. In another embodiment, R₁, R₂, R₃, R₄, R₅ and R₆ are independently C(O)O-alkyl. In another embodiment, R₁, R₂, R₃, R₄, R₅ and R₆ are independently C(O)H. In another embodiment, R₁, R₂, R₃, R₄, R₅ and R₆ are independently —C(O)NH₂. In another embodiment, R₁, R₂, R₃, R₄, R₅ and R₆ are independently NO₂.

The invention also encompasses methods of treating pancreatic cancer by administering at least one compound of formula XII in a therapeutically effective amount to a subject in need thereof, wherein the compound of Formula XII is represented by the structure:

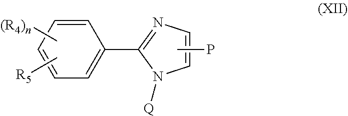

wherein,
P and Q are independently H or

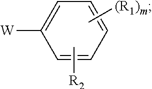

### W is C═O, C═S, SO₂ or S═O;

wherein at least one of Q or P is not hydrogen;
R₁ and R₄ are independently H, O-alkyl, I, Br, Cl, F, alkyl, haloalkyl, aminoalkyl, OCH₂Ph, OH, CN, NO₂, —NHCO-alkyl, COOH, —(CH₂)ᵢNHCH₃, —(CH₂)ᵢNH₂, —(CH₂)ᵢN(CH₃)₂; C(O)O-alkyl or C(O)H; wherein at least one of R₁ and R₄ is not hydrogen;
R₂ and R₅ are independently H, O-alkyl, I, Br, Cl, F, alkyl, haloalkyl, aminoalkyl, OCH₂Ph, OH, CN, NO₂, —NHCO-alkyl, COOH, C(O)O-alkyl or C(O)H;
m is an integer between 1-4;
i is an integer between 0-5; and
n is an integer between 1-4;
or its pharmaceutically acceptable salt, hydrate, polymorph, metabolite, tautomer or isomer.

The invention also encompasses methods of treating pancreatic cancer by administering at least one compound of formula XIII in a therapeutically effective amount to a subject in need thereof, wherein the compound of Formula XIII is represented by the structure:

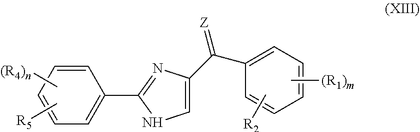

wherein

### Z is O or S;

R₁ and R₄ are independently H, O-alkyl, I, Br, Cl, F, alkyl, haloalkyl, aminoalkyl, OCH₂Ph, OH, CN, NO₂, —NHCO-alkyl, haloalkyl, aminoalkyl, —(CH₂)ᵢNHCH₃, —(CH₂)ᵢNH₂, —(CH₂)ᵢN(CH₃)₂; COOH, C(O)O-alkyl or C(O)H; wherein at least one of R₁ and R₄ is not hydrogen;
R₂ and R₅ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂; OCH₂Ph, OH, CN, NO₂, —NHCO-alkyl, COOH, C(O)O-alkyl or C(O)H;
m is an integer between 1-4;
i is an integer between 0-5; and
n is an integer between 1-4;
or its pharmaceutically acceptable salt, hydrate, polymorph, metabolite, tautomer or isomer.

The invention also encompasses methods of treating pancreatic cancer by administering at least one compound of formula (XIV) in a therapeutically effective amount to a subject in need thereof, wherein the compound of Formula (XIV) is represented by the structure:

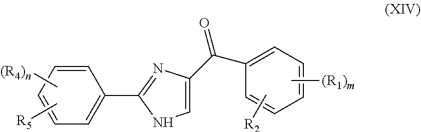

wherein R₁ and R₄ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H; wherein at least one of R₁ and R₄ is not hydrogen;
R₂ and R₅ are independently H, O-alkyl, I, Br, Cl, F, alkyl, haloalkyl, aminoalkyl, OCH₂Ph, OH, CN, NO₂, —NHCO-alkyl, COOH, C(O)O-alkyl or C(O)H;
m is an integer between 1-4;
i is an integer between 0-5; and
n is an integer between 1-4;
or its pharmaceutically acceptable salt, hydrate, polymorph, metabolite, tautomer or isomer.

In one embodiment, R₁ of compound of formula XII, XIII and XIV is OCH₃. In another embodiment, R₁ of compound of formula XII, XIII and XIV is 4-F. In another embodiment, R₁ of compound of formula XII, XIII and XIV is OCH₃ and m is 3. In another embodiment, R₄ of compound of formula XII, XIII and XIV is 4-F. In another embodiment, R₄ of compound of formula XII, XIII and XIV is OCH₃. In another embodiment, R₄ of compound of formula XIV is CH₃. In another embodiment, R₄ of compound of formula XII, XIII and XIV is 4-Cl. In another embodiment, R₄ of compound of formula XII, XIII and XIV is 4-N(Me)₂. In another embodiment, R₄ of compound of formula XII, XIII and XIV is OBn. In another embodiment, R₄ of compound of formula XII, XIII and XIV is 4-Br. In another embodiment, R₄ of compound of formula XII, XIII and XIV is 4-CF₃. Non limiting examples of compounds of formula XIV are selected from: (2-phenyl-1H-imidazol-4-yl)(3,4,5-trimethoxyphenyl)methanone (12aa), (4-fluorophenyl)(2-phenyl-1H-imidazol-4-yl)methanone (12af), (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (4-fluorophenyl)(2-(4-methoxyphenyl)-1H-imidazol-4-yl)methanone (12cb), (2-(p-tolyl)-1H-imidazol-4-yl)(3,4,5-trimethoxyphenyl)methanone (12da), (4-fluorophenyl)(2-(p-tolyl)-1H-imidazol-4-yl)methanone (12db), (4-hydroxy-3,5-dimethoxyphenyl)(2-(p-tolyl)-1H-imidazol-4-yl)methanone (12dc), (2-(4-chlorophenyl)-1H-imidazol-4-yl)(3,4,5-trimethoxyphenyl)methanone (12fa), (2-(4-chlorophenyl)-1H-imidazol-4-yl)(4-fluorophenyl)methanone (12fb), (2-(4-chlorophenyl)-1H-imidazol-4-yl)(4-hydroxy-3,5-dimethoxyphenyl)methanone (12fc), (2-(4-(dimethylamino)phenyl)-1H-imidazol-4-yl)(3,4,5-trimethoxyphenyl)methanone (12ga); (2-(4-(dimethylamino)phenyl)-1H-imidazol-4-yl)(4-fluorophenyl)methanone (12gb), (2-(3,4-dimethoxyphenyl)-1H-imidazol-4-yl)(3,4,5-trimethoxyphenyl)methanone (12ha), (2-(4-(benzyloxy)phenyl)-1H-imidazol-4-yl)(4-fluorophenyl)methanone (12jb), (2-(4-bromophenyl)-1H-imidazol-4-yl)(3,4,5-trimethoxyphenyl)methanone (121a), (2-(4-(trifluoromethyl)phenyl)-1H-imidazol-4-yl)(3,4,5-trimethoxyphenyl)methanone (12pa).

The invention also encompasses methods of treating pancreatic cancer by administering at least one compound of formula (XIVa) in a therapeutically effective amount to a subject in need thereof, wherein the compound of Formula (XIVa) is represented by the structure:

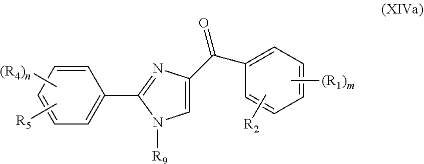

wherein R₁ and R₄ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO, —NHCO-alkyl, COOH, C(O)O-alkyl or C(O)H; wherein at least one of R₁ and R₄ is not hydrogen; R₂ and R₅ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO, —NHCO-alkyl, COOH, C(O)O-alkyl or C(O)H; R₉ is H, linear or branched, substituted or unsubstituted alkyl, substituted or unsubstituted aryl, CH₂Ph, substituted benzyl, haloalkyl, aminoalkyl, OCH₂Ph, substituted or unsubstituted SO₂-Aryl, substituted or unsubstituted —(C═O)-Aryl or OH;
wherein substitutions are independently selected from the group of hydrogen (e.g., no substitution at a particular position), hydroxyl, an aliphatic straight- or branched-chain C₁ to C₁₀ hydrocarbon, alkoxy, haloalkoxy, aryloxy, nitro, cyano, alkyl-CN, halo (e.g., F, Cl, Br, I), haloalkyl, dihaloalkyl, trihaloalkyl, COOH, C(O)Ph, C(O)-alkyl, C(O)O— alkyl, C(O)H, C(O)NH₂, —OC(O)CF₃, OCH₂Ph, amino, aminoalkyl, alkylamino, mesylamino, dialkylamino, arylamino, amido, NHC(O)-alkyl, urea, alkyl-urea, alkylamido (e.g., acetamide), haloalkylamido, arylamido, aryl, and C₅ to C₇ cycloalkyl, arylalkyl, and combinations thereof;
m is an integer between 1-4;
i is an integer between 0-5; and
n is an integer between 1-4;
or its pharmaceutically acceptable salt, hydrate, polymorph, metabolite, tautomer or isomer.

In one embodiment, R₉ of compound of formula XVa is CH₃. In another embodiment, R₉ of compound of formula XIVa is CH₂Ph. In another embodiment, R₉ of compound of formula XIVa is (SO₂)Ph. In another embodiment, R₉ of compound of formula XIVa is (SO₂)-Ph-OCH₃. In another embodiment, R₉ of compound of formula XIVa is H. In another embodiment, R₄ of compound of formula XIVa is H. In another embodiment, R₄ of compound of formula XIVa is CH₃. In another embodiment, R₄ of compound of formula XIVa is OCH₃. In another embodiment, R₄ of compound of formula XIVa is OH. In another embodiment, R₄ of compound of formula XIVa is 4-Cl. In another embodiment, R₄ of compound of formula XIVa is 4-N(Me)₂. In another embodiment, R₄ of compound of formula XIVa is OBn. In another embodiment, R₁ of compound of formula XIVa is OCH₃; m is 3 and R₂ is H. In another embodiment, R₁ of compound of formula XIVa is F; m is 1 and R₂ is H. Non limiting examples of compounds of formula XIVa are selected from: (4-fluorophenyl)(2-phenyl-1-(phenylsulfonyl)-1H-imidazol-4-yl)methanone (11af), (4-fluorophenyl)(2-(4-methoxyphenyl)-1-(phenylsulfonyl)-1H-imidazol-4-yl)methanone (11cb), (4-fluorophenyl)(1-(phenylsulfonyl)-2-(p-tolyl)-1H-imidazol-4-yl)methanone (11db), (2-(4-chlorophenyl)-1-(phenylsulfonyl)-1H-imidazol-4-yl)(4-fluorophenyl)methanone (11fb), (2-(4-(dimethylamino)phenyl)-1-(phenysulfonyl)-1H-imidazol-4-yl)(3,4,5-trimethoxyphenyl)methanone (11ga), (2-(4-(dimethylamino)phenyl)-1-(phenylsulfonyl)-1H-imidazol-4-yl)(4-fluorophenyl)methanone (11gb), (2-(3,4-dimethoxyphenyl)-1-(phenylsulfonyl)-1H-imidazol-4-yl)(3,4,5-trimethoxyphenyl)methanone (11ha), (2-(4-(benzyloxy)phenyl)-1-(phenylsulfonyl)-1H-imidazol-4-yl)(4-fluorophenyl)methanone (11jb), (2-(4-(dimethylamino)phenyl)-1-((4-methoxyphenyl)sulfonyl)-1H-imidazol-4-yl)(4-fluorophenyl)methanone (12gba), (1-benzyl-2-(p-tolyl)-1H-imidazol-4-yl)(3,4,5-trimethoxyphenyl)methanone (12daa), (1-methyl-2-(p-tolyl)-1H-imidazol-4-yl)(3,4,5-trimethoxyphenyl)methanone (12dab), (4-fluorophenyl)(2-(4-methoxyphenyl)-1-methyl-1H-imidazol-4-yl)methanone (12cba).

The invention also encompasses methods of treating pancreatic cancer by administering at least one compound of formula (XV) in a therapeutically effective amount to a subject in need thereof, wherein the compound of Formula (XV) is represented by the structure:

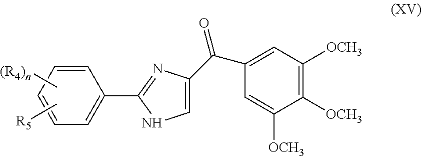

wherein R₄ and R₅ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H;
i is an integer between 0-5; and
n is an integer between is 1-4;
or its pharmaceutically acceptable salt, hydrate, polymorph, metabolite, tautomer or isomer.

In one embodiment, R₄ of compound of formula XV is H. In another embodiment, R₄ of compound of formula XV is F. In another embodiment, R₄ of compound of formula XV is Cl. In another embodiment, R₄ of compound of formula XV is Br. In another embodiment, R₄ of compound of formula XV is I. In another embodiment, R₄ of compound of formula XV is N(Me)₂. In another embodiment, R₄ of compound of formula XV is OBn. In another embodiment, R₄ of compound of formula XV is OCH₃. In another embodiment, R₄ of compound of formula XV is CH₃. In another embodiment, R₄ of compound of formula XV is CF₃. Non limiting examples of compounds of formula XV are selected from: (2-phenyl-1H-imidazol-4-yl)(3,4,5-trimethoxyphenyl)methanone (12aa), (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (2-(p-tolyl)-1H-imidazol-4-yl)(3,4,5-trimethoxyphenyl)methanone (12da), (3,4,5-trimethoxyphenyl)(2-(3,4,5-trimethoxyphenyl)-1H-imidazol-4-yl)methanone (12ea), (2-(4-chlorophenyl)-1H-imidazol-4-yl)(3,4,5-trimethoxyphenyl)methanone (12fa), (2-(4-(dimethylamino)phenyl)-1H-imidazol-4-yl)(3,4,5-trimethoxyphenyl)methanone (12ga), (2-(3,4-dimethoxyphenyl)-1H-imidazol-4-yl)(3,4,5-trimethoxyphenyl)methanone (12ha), (2-(2-(trifluoromethyl)phenyl)-1H-imidazol-4-yl)(3,4,5-trimethoxyphenyl)methanone (121a), (2-(4-(benzyloxy)phenyl)-1H-imidazol-4-yl)(3,4,5-trimethoxyphenyl)methanone (12ja), (2-(4-hydroxyphenyl)-1H-imidazol-4-yl)(3,4,5-trimethoxyphenyl)methanone (12ka), (2-(4-bromophenyl)-1H-imidazol-4-yl)(3,4,5-trimethoxyphenyl)methanone (121a), (2-(4-(trifluoromethyl)phenyl)-1H-imidazol-4-yl)(3,4,5-trimethoxyphenyl)methanone (12pa).

The invention also encompasses methods of treating pancreatic cancer by administering at least one compound of formula (XVI) in a therapeutically effective amount to a subject in need thereof, wherein the compound of Formula (XVI) is represented by the structure:

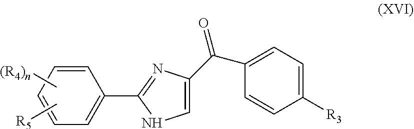

wherein R₄ and R₅ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H;

### R₃ is I, Br, Cl, or F;

i is an integer between 0-5; and
n is an integer between 1-4;
or its pharmaceutically acceptable salt, hydrate, polymorph, metabolite, tautomer or isomer.

In one embodiment, R₃ of compound of formula XVI is halogen. In another embodiment, R₃ is F. In another embodiment, R₃ is Cl. In another embodiment R₃ is Br. In another embodiment R₃ is I. In another embodiment R₄ is H. In another embodiment R₄ is OCH₃. In another embodiment R₄ is OCH₃; n is 3 and R₅ is H. In another embodiment R₄ is CH₃. In another embodiment R₄ is F. In another embodiment R₄ is Cl. In another embodiment R₄ is Br. In another embodiment R₄ is I. In another embodiment R₄ is N(Me)₂. In another embodiment R₄ is OBn. In another embodiment, R₃ is F; R₅ is hydrogen; n is 1 and R₄ is 4-Cl. In another embodiment, R₃ is F; R₅ is hydrogen; n is 1 and R₄ is 4-OCH₃. In another embodiment, R₃ is F; R₅ is hydrogen; n is 1 and R₄ is 4-CH₃. In another embodiment, R₃ is F; R₅ is hydrogen; n is 1 and R₄ is 4-N(Me)₂. In another embodiment, R₃ is F; R₅ is hydrogen; n is 1 and R₄ is 4-OBn. Non limiting examples of compounds of formula XVI are selected from: (4-fluorophenyl)(2-phenyl-1H-imidazol-4-yl)methanone (12af), (4-fluorophenyl)(2-(4-methoxyphenyl)-1H-imidazol-4-yl)methanone (12cb), (4-fluorophenyl)(2-(p-tolyl)-1H-imidazol-4-yl)methanone (12db), 4-fluorophenyl)(2-(3,4,5-trimethoxyphenyl)-1H-imidazol-4-yl)methanone (12eb), (2-(4-chlorophenyl)-1H-imidazol-4-yl)(4-fluorophenyl)methanone (12fb), (2-(4-(dimethylamino)phenyl)-1H-imidazol-4-yl)(4-fluorophenyl)methanone (12gb), (2-(4-(benzyloxy)phenyl)-1H-imidazol-4-yl)(4-fluorophenyl)methanone (12jb).

The invention also encompasses methods of treating pancreatic cancer by administering at least one compound of formula (XVII) in a therapeutically effective amount to a subject in need thereof, wherein the compound of Formula (XVII) is represented by the structure:

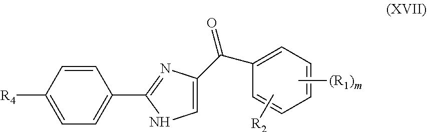

wherein R₄ is H, O-alkyl, I, Br, Cl, F, alkyl, haloalkyl, aminoalkyl, OCH₂Ph, OH, CN, NO₂, —NHCO-alkyl, COOH, C(O)O— alkyl or C(O)H;
wherein R₁ and R₂ are independently H, O-alkyl, I, Br, Cl, F, alkyl, haloalkyl, aminoalkyl, OCH₂Ph, OH, CN, NO₂, —NHCO-alkyl, COOH, C(O)O-alkyl or C(O)H;
and
m is an integer between 1-4;
or its pharmaceutically acceptable salt, hydrate, polymorph, metabolite, tautomer or isomer.
In one embodiment, R₄ of compound of formula XVII is halogen. In another embodiment, R₄ is F. In another embodiment, R₄ is Cl. In another embodiment R₄ is Br. In another embodiment R₄ is I. In another embodiment, R₄ is OCH₃. In another embodiment, R₄ is CH₃. In another embodiment, R₄ is N(Me)₂. In another embodiment, R₄ is CF₃. In another embodiment, R₄ is OH. In another embodiment, R₄ is OBn. In another embodiment, R₁ of compound of formula XVII is halogen. In another embodiment, R₁ of compound of formula XVII is F. In another embodiment, R₁ of compound of formula XVII is Cl. In another embodiment, R₁ of compound of formula XVII is Br. In another embodiment, R₁ of compound of formula XVII is I. In another embodiment, R₁ of compound of formula XVII is OCH₃. In another embodiment, R₁ of compound of formula XVII is OCH₃, m is 3 and R₂ is H. In another embodiment, R₁ of compound of formula XVII is F, m is 1 and R₂ is H. In another embodiment, R₄ is F; R₂ is hydrogen; n is 3 and R₁ is OCH₃. In another embodiment, R₄ is OCH₃; R₂ is hydrogen; n is 3 and R₁ is OCH₃. In another embodiment, R₄ is CH₃; R₂ is hydrogen; n is 3 and R₁ is OCH₃. In another embodiment, R₄ is Cl; R₂ is hydrogen; n is 3 and R₁ is OCH₃. In another embodiment, R₄ is N(Me)₂; R₂ is hydrogen; n is 3 and R₁ is OCH₃. In one embodiment, R₄ of compound of formula XVII is halogen, R₁ is H and R₂ is halogen. In one embodiment, R₄ of compound of formula XVII is halogen, R₁ is halogen and R₂ is H. In one embodiment, R₄ of compound of formula XVII is alkoxy, R₁ is halogen and R₂ is H. In one embodiment, R₄ of compound of formula XVII is methoxy, R₁ is halogen and R₂ is H. Non limiting examples of compounds of formula XVII are selected from: (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (4-fluorophenyl)(2-(4-methoxyphenyl)-1H-imidazol-4-yl)methanone (12cb), (2-(p-tolyl)-1H-imidazol-4-yl)(3,4,5-trimethoxyphenyl)methanone (12da), (4-fluorophenyl)(2-(p-tolyl)-1H-imidazol-4-yl)methanone (12db), (4-Hydroxy-3,5-dimethoxyphenyl)(2-(p-tolyl)-1H-imidazol-4-yl)methanone (12dc), (2-(4-chlorophenyl)-1H-imidazol-4-yl)(3,4,5-trimethoxyphenyl)methanone (12fa), (2-(4-chlorophenyl)-1H-imidazol-4-yl)(4-fluorophenyl)methanone (12fb), (2-(4-chlorophenyl)-1H-imidazol-4-yl)(3,4,5-trihydroxyphenyl)methanone (13fa), (2-(4-(dimethylamino)phenyl)-1H-imidazol-4-yl)(3,4,5-trimethoxyphenyl)methanone (12ga), (2-(4-(dimethylamino)phenyl)-1H-imidazol-4-yl)(4-fluorophenyl)methanone (12gb), (2-(4-(benzyloxy)phenyl)-1H-imidazol-4-yl)(4-fluorophenyl)methanone (12jb), (2-(4-hydroxyphenyl)-1H-imidazol-4-yl)(3,4,5-trimethoxyphenyl)methanone (12ka), (2-(4-bromophenyl)-1H-imidazol-4-yl)(3,4,5-trimethoxyphenyl)methanone (121a), (2-(4-(trifluoromethyl)phenyl)-1H-imidazol-4-yl)(3,4,5-trimethoxyphenyl)methanone (12pa).

In another embodiment a compound of formula XVII is represented by the structure of formula 12fb:

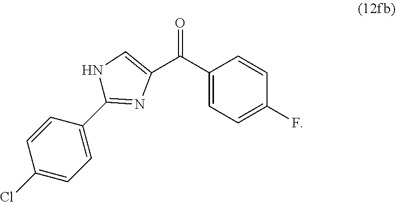 [SMILES: O=C(c1ccc(F)cc1)c1c[nH]c(-c2ccc(Cl)cc2)n1]

In another embodiment a compound of formula XVII is represented by the structure of formula 12cb:

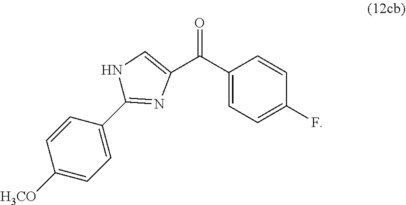 [SMILES: COc1ccc(-c2nc(C(=O)c3ccc(F)cc3)c[nH]2)cc1]

The invention also encompasses methods of treating pancreatic cancer by administering at least one compound of formula (XVIII) in a therapeutically effective amount to a subject in need thereof, wherein the compound of Formula (XVIII) is represented by the structure:

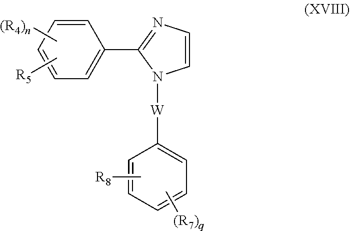

wherein

### W is C═O, C═S, SO₂ or S═O;

R₄ and R₇ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H;
R₅ and R₈ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H;
n is an integer between 1-4;
i is an integer between 0-5; and
q is an integer between 1-4;
or its pharmaceutically acceptable salt, hydrate, polymorph, metabolite, tautomer or isomer.

In one embodiment, W of compound of formula XVIII is C═O. In another embodiment, W of compound of formula XVIII is SO₂. In another embodiment, R₄ of compound of formula XVIII is H. In another embodiment, R₄ of compound of formula XVIII is NO₂. In another embodiment, R₄ of compound of formula XVIII is OBn. In another embodiment, R₇ of compound of formula XVIII is H. In another embodiment, R₇ of compound of formula XVIII is OCH₃. In another embodiment, R₇ of compound of formula XVIII is OCH₃ and q is 3. Non limiting examples of compounds of formula XVII are selected from: (4-methoxyphenyl)(2-phenyl-1H-imidazol-1-yl)methanone (12aba), (2-phenyl-1H-imidazol-1-yl)(3,4,5-trimethoxyphenyl)methanone (12aaa), 2-phenyl-1-(phenylsulfonyl)-1H-imidazole (10a), 2-(4-nitrophenyl)-1-(phenylsulfonyl)-1H-imidazole (10x), 2-(4-(benzyloxy)phenyl)-1-(phenylsulfonyl)-1H-imidazole (10j).

The invention also encompasses methods of treating pancreatic cancer by administering at least one compound of formula (XIX) in a therapeutically effective amount to a subject in need thereof, wherein the compound of Formula (XIX) is represented by the structure:

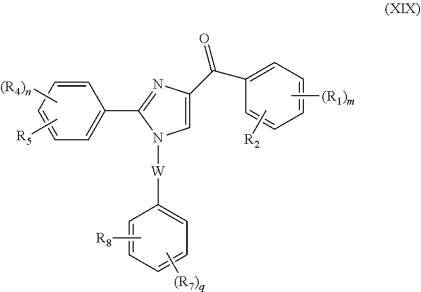

wherein

### W is C═O, C═S, SO₂, S═O;

R₁, R₄ and R₇ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H;
R₂, R₅ and R₆ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H;
m is an integer between 1-4;
n is an integer between 1-4;
i is an integer between 0-5; and
q is 1-4;
or its pharmaceutically acceptable salt, hydrate, polymorph, metabolite, tautomer or isomer.

In one embodiment, R₁, R₄ and R₇ of formula XIX are independently H. In another embodiment, R₂, R₄ and R₇ of formula XIX are independently O-alkyl. In another embodiment, R₁, R₄ and R₇ of formula XIX are independently halogen. In another embodiment, R₁, R₄ and R₇ of formula XIX are independently CN. In another embodiment, R₁, R₄ and R₇ of formula XIX are independently OH. In another embodiment, R₁, R₄ and R₇ of formula XIX are independently alkyl. In another embodiment, R₁, R₄ and R₇ of formula XIX are independently OCH₂Ph. In one embodiment R₂, R₅ and R₈ of formula XIX are independently H. In another embodiment, R₂, R₅ and R₈ of formula XIX are independently O-alkyl. In another embodiment, R₂, R₅ and R₈ of formula XIX are independently halogen. In another embodiment, R₂, R₅ and R₈ of formula XIX are independently CN. In another embodiment, R₂, R₅ and R₈ of formula XIX are independently OH. In another embodiment, R₂, R₅ and R₈ of formula XIX are independently alkyl. In another embodiment, R₂, R₅ and R₈ of formula XIX are independently OCH₂Ph. In another embodiment, R₅, R₂ and R₈ of formula XIX are H, R₄ is 4-N(Me)₂, R₁ is OCH₃, m is 3 and R₇ is OCH₃. In another embodiment, R₅, R₂, R₇ and R₈ of formula XIX are H, R₄ is 4-Br, R₁ is OCH₃, and m is 3. In another embodiment W is SO₂. In another embodiment W is C═O. In another embodiment W is C═S. In another embodiment W is S═O. Non limiting examples of compounds of formula XIX are selected from: (2-(4-(dimethylamino)phenyl)-1-((4-methoxyphenyl)sulfonyl)-1H-imidazo-4-yl)(3,4,5-trimethoxyphenyl)methanone (11gaa); (2-(4-bromophenyl)-1-(phenylsulfonyl)-1H-imidazol-4-yl)(3,4,5-trimethoxyphenyl)methanone (111a), (4-fluorophenyl)(2-(4-methoxyphenyl)-1-(phenylsulfonyl)-1H-imidazol-4-yl)methanone (11cb), (2-(4-chlorophenyl)-1-(phenylsulfonyl)-1H-imidazol-4-yl)(4-fluorophenyl)methanone (11fb), (4-fluorophenyl)(2-phenyl-1-(phenylsulfonyl)-1H-imidazol-4-yl)methanone (11af), (4-fluorophenyl)(1-(phenylsulfonyl)-2-(p-tolyl)-1H-imidazol-4-yl)methanone (11db), (2-(4-(dimethylamino)phenyl)-1-(phenylsulfonyl)-1H-imidazol-4-yl)(3,4,5-trimethoxyphenyl)methanone (11ga), (2-(4-(dimethylamino)phenyl)-1-(phenylsulfonyl)-1H-imidazol-4-yl)(4-fluorophenyl)methanone (11gb), (2-(3,4-dimethoxyphenyl)-1-(phenylsulfonyl)-1H-imidazol-4-yl)(3,4,5-trimethoxyphenyl)methanone (11ha), (2-(4-(benzyloxy)phenyl)-1-(phenylsulfonyl)-1H-imidazol-4-yl)(4-fluorophenyl)methanone (11jb), (2-(4-(dimethylamino)phenyl)-1-((4-methoxyphenyl)sulfonyl)-1H-imidazol-4-yl)(4-fluorophenyl)methanone (12gba).

In another embodiment a compound of formula XIX is represented by the structure of formula 11cb:

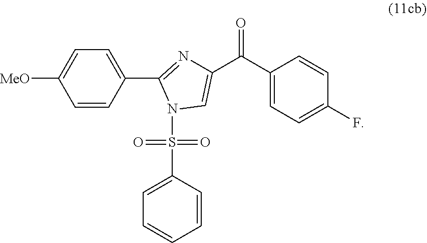 [SMILES: COc1ccc(-c2nc(C(=O)c3ccc(F)cc3)cn2S(=O)(=O)c2ccccc2)cc1]

In another embodiment a compound of formula XIX is represented by the structure of formula 11fb:

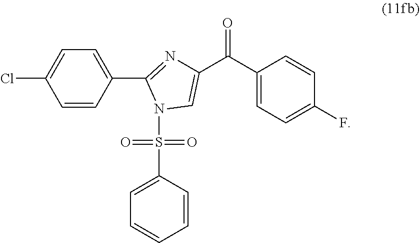 [SMILES: O=C(c1ccc(F)cc1)c1cn(S(=O)(=O)c2ccccc2)c(-c2ccc(Cl)cc2)n1]

The invention also encompasses methods of treating pancreatic cancer by administering at least one compound of formula (XX) in a therapeutically effective amount to a subject in need thereof, wherein the compound of Formula (XX) is represented by the structure:

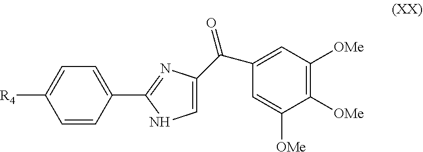

wherein
R₄ is H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H; and
i is an integer between 0-5;
or its pharmaceutically acceptable salt, hydrate, polymorph, metabolite, tautomer or isomer.

In one embodiment, R₄ of compound of formula XX is H. In another embodiment, R₄ of compound of formula XX is halogen. In another embodiment, R₄ is F. In another embodiment, R₄ is Cl. In another embodiment R₄ is Br. In another embodiment R₄ is I. In another embodiment, R₄ is alkyl. In another embodiment, R₄ is methyl. Non limiting examples of compounds of formula XX are selected from: (2-phenyl-1H-imidazol-4-yl)(3,4,5-trimethoxyphenyl)methanone (12aa), (2-(4-fluorophenyl)-1H-imidazol-4-yl)(3,4,5-trimethoxyphenyl)methanone (12ba), (2-(4-methoxyphenyl)-1H-imidazol-4-yl)(3,4,5-trimethoxyphenyl)methanone (12ca), (2-(p-tolyl)-1H-imidazol-4-yl)(3,4,5-trimethoxyphenyl)methanone (12da), (2-(4-chlorophenyl)-1H-imidazol-4-yl)(3,4,5-trimethoxyphenyl)methanone (12fa), (2-(4-(dimethylamino)phenyl)-1H-imidazol-4-yl)(3,4,5-trimethoxyphenyl)methanone (12ga), (2-(2-(trifluoromethyl)phenyl)-1H-imidazol-4-yl)(3,4,5-trimethoxyphenyl)methanone (121a), (2-(4-(benzyloxy)phenyl)-1H-imidazol-4-yl)(3,4,5-trimethoxyphenyl)methanone (12ja), (2-(4-hydroxyphenyl)-1H-imidazol-4-yl)(3,4,5-trimethoxyphenyl)methanone (12ka), (2-(4-bromophenyl)-1H-imidazol-4-yl)(3,4,5-trimethoxyphenyl)methanone (121a), (2-(4-(trifluoromethyl)phenyl)-1H-imidazol-4-yl)(3,4,5-trimethoxyphenyl)methanone (12pa).

In another embodiment a compound of formula XX is represented by the structure of formula 12da:

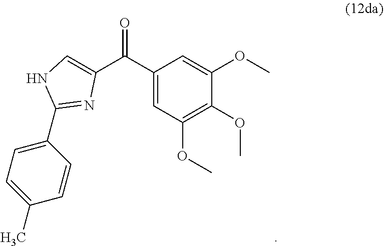 [SMILES: COc1cc(C(=O)c2c[nH]c(-c3ccc(C)cc3)n2)cc(OC)c1OC]

In another embodiment a compound of formula XX is represented by the structure of formula 12fa:

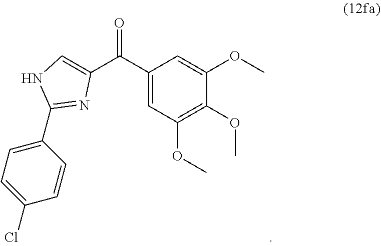 [SMILES: COc1cc(C(=O)c2c[nH]c(-c3ccc(Cl)cc3)n2)cc(OC)c1OC]

The invention also encompasses methods of treating pancreatic cancer by administering at least one compound of formula (XXI) in a therapeutically effective amount to a subject in need thereof, wherein the compound of Formula (XXI) is represented by the structure:

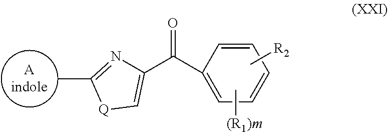

wherein
A is indolyl;

### Q is NH, O or 5;

R₁ and R₂ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H; and
wherein said A is optionally substituted by substituted or unsubstituted O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, substituted or unsubstituted —SO₂-aryl, substituted or unsubstituted C₁-C₅ linear or branched alkyl, substituted or unsubstituted haloalkyl, substituted or unsubstituted alkylamino, substituted or unsubstituted aminoalkyl, —OCH₂Ph, substituted or unsubstituted —NHCO-alkyl, COOH, substituted or unsubstituted —C(O)Ph, substituted or unsubstituted C(O)O-alkyl, C(O)H, —C(O)NH₂, NO₂ or combination thereof;
i is an integer between 0-5; and
m is an integer between 1-4;
or its pharmaceutically acceptable salt, hydrate, polymorph, metabolite, tautomer or isomer.

In one embodiment, R₁ of compound of formula XXI is OCH₃; m is 3 and R₂ is hydrogen. In another embodiment, R₁ is F; m is 1 and R₂ is hydrogen. In one embodiment, Q of formula XXI is O. In another embodiment Q of formula XXI is NH. In another embodiment, Q of formula XXI is S.

In one embodiment, A ring of compound of formula XXI is substituted 5-indolyl. In another embodiment the substitution is —(C═O)-Aryl. In another embodiment, the aryl is 3,4,5-(OCH₃)₃-Ph.

In another embodiment, A ring of compound of formula XXI is 3-indolyl. In another embodiment, A ring of compound of formula XXI is 5-indolyl. In another embodiment, A ring of compound of formula XXI is 2-indolyl. Non limiting examples of compounds of formula XXI are selected from: (5-(4-(3,4,5-trimethoxybenzoyl)-1H-imidazol-2-yl)-1H-indol-2-yl)(3,4,5-trimethoxyphenyl)methanone (15xaa); (1-(phenylsulfonyl)-2-(1-(phenylsulfonyl)-2-(3,4,5-trimethoxybenzoyl)-1H-indol-5-yl)-1H-imidazol-4-yl)(3,4,5-trimethoxyphenyl)methanone (16xaa); 2-(1H-indol-3-yl)-1H-imidazol-4-yl)(3,4,5-trimethoxyphenyl)methanone (17ya); (2-(1H-indol-2-yl)thiazol-4-yl)(3,4,5-trimethoxyphenyl)methanone (62a); and (2-(1H-indol-5-yl)thiazol-4-yl)(3,4,5-trimethoxyphenyl)methanone (66a).

The invention also encompasses methods of treating pancreatic cancer by administering at least one compound of formula (XXIa) in a therapeutically effective amount to a subject in need thereof, wherein the compound of Formula (XXIa) is represented by the structure:

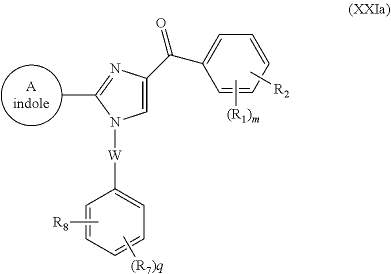

wherein

### W is C═O, C═S, SO₂, S═O;

A is indolyl;
R₁ and R₂ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H;
R₇ and R₈ are independently H, O-alkyl, I, Br, Cl, F, alkyl, haloalkyl, aminoalkyl, —(CH₂)ᵢNHCH₃, —(CH₂)ᵢNH₂, —(CH₂)ᵢN(CH₃)₂, OCH₂Ph, OH, CN, NO₂, —NHCO-alkyl, COOH, C(O)O-alkyl or C(O)H; wherein said A is optionally substituted by substituted or unsubstituted O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, substituted or unsubstituted —SO₂-aryl, substituted or unsubstituted C₁-C₅ linear or branched alkyl, substituted or unsubstituted haloalkyl, substituted or unsubstituted alkylamino, substituted or unsubstituted aminoalkyl, —OCH₂Ph, substituted or unsubstituted —NHCO-alkyl, COOH, substituted or unsubstituted —C(O)Ph, substituted or unsubstituted C(O)O-alkyl, C(O)H, —C(O)NH₂, NO₂ or combination thereof;
i is an integer between 0-5; and
m is an integer between 1-4;
q is an integer between 1-4;
or its pharmaceutically acceptable salt, hydrate, polymorph, metabolite, tautomer or isomer.

In one embodiment, R₁ of compound of formula XXIa is OCH₃; m is 3 and R₂ is hydrogen. In another embodiment, R₁ is F; m is 1 and R₂ is hydrogen. In another embodiment, A ring of compound of formula XXIa is substituted 5-indolyl. In another embodiment, A ring of compound of formula XXIa is 3-indolyl. Non limiting examples of compounds of formula XXIa are selected from: (1-(phenylsulfonyl)-2-(1-(phenylsulfonyl)-2-(3,4,5-trimethoxybenzoyl)-1H-indol-5-yl)-1H-imidazol-4-yl)(3,4,5-trimethoxyphenyl)methanone (16xaa); (1-(phenylsulfonyl)-2-(1-(phenylsulfonyl)-1H-indol-3-yl)-1H-imidazol-4-yl)(3,4,5-trimethoxyphenyl)methanone (17yaa).

The invention also encompasses methods of treating pancreatic cancer by administering at least one compound of formula (XXII) in a therapeutically effective amount to a subject in need thereof, wherein the compound of Formula (XXII) is represented by the structure:

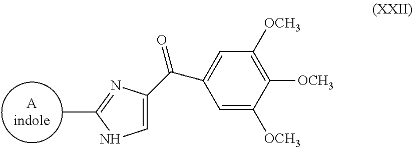

wherein
A is indolyl;
wherein said A is optionally substituted by substituted or unsubstituted O-alkyl, O-haloalkyl, F, Cl, Br, I, haloalkyl, CF₃, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, substituted or unsubstituted —SO₂-aryl, substituted or unsubstituted C₁-C₅ linear or branched alkyl, substituted or unsubstituted haloalkyl, substituted or unsubstituted alkylamino, substituted or unsubstituted aminoalkyl, —OCH₂Ph, substituted or unsubstituted —NHCO-alkyl, COOH, substituted or unsubstituted —C(O)Ph, substituted or unsubstituted C(O)O-alkyl, C(O)H, —C(O)NH₂, NO₂ or combination thereof;
i is an integer between 0-5;
or its pharmaceutically acceptable salt, hydrate, polymorph, metabolite, tautomer or isomer.

In one embodiment, A ring of compound of formula XXII is substituted 5-indolyl. In another embodiment the substitution is —(C═O)-Aryl. In another embodiment, the aryl is 3,4,5-(OCH₃)₃-Ph.

In another embodiment, A ring of compound of formula XXII is 3-indolyl. Non limiting examples of compounds of formula XXII are selected from: (5-(4-(3,4,5-trimethoxybenzoyl)-1H-imidazol-2-yl)-1H-indol-2-yl)(3,4,5-trimethoxyphenyl)methanone (15xaa); (2-(1H-indol-3-yl)-1H-imidazol-4-yl)(3,4,5-trimethoxyphenyl)methanone (17ya),

In another embodiment a compound of formula XXI or XXII is represented by the structure of formula 17ya:

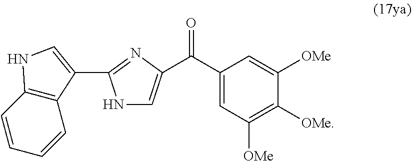 [SMILES: COc1cc(C(=O)c2c[nH]c(-c3c[nH]c4ccccc34)n2)cc(OC)c1OC]

In one embodiment, Q of compound of formula XII is H and P is

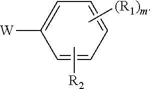

In another embodiment, P of compound of formula XII is H and Q is

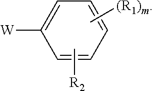

In another embodiment, P of compound of formula XII is

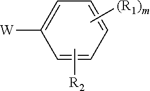

and Q is SO₂-Ph. In one embodiment. Q of compound of formula XII is H and P is

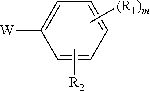

wherein W is C═O. In another embodiment W of compound of formula XII, XVIII, XIX, or XXIa is C═O. In another embodiment, W of compound of formula XII, XVIII, XIX, or XXIa is SO₂. In another embodiment, W of compound of formula XII, XVIII, XIX, or XXIa is C═S. In another embodiment, W of compound of formula XII, XVIII, XIX, or XXIa is S═O.

In one embodiment, Z of compound of formula XIII is oxygen. In another embodiment, Z of compound of formula XIII is sulfur.

In one embodiment, R₄ of compound of formula XII-XVI, XVIII, or XIX is hydrogen, n is 1 and R₄ is in the para position.

In one embodiment, R₄ of compound of formula XII-XX is alkyl. In another embodiment, R₄ of compound of formula XII-XX is H. In another embodiment, R₄ of compound of formula XII-XX is methyl (CH₃). In another embodiment, R₄ of compound of formula XII-XX is O-alkyl. In another embodiment, R₄ of compound of formula XII-XX is OCH₃. In another embodiment, R₄ of compound of formula XII-XX is I. In another embodiment, R₄ of compound of formula XII-XX is Br. In another embodiment, R₄ of compound of formula XII-XX is F. In another embodiment, R₄ of compound of formula XII-XX is Cl. In another embodiment, R₄ of compound of formula XII-XX is N(Me)₂. In another embodiment, R₄ of compound of formula XI-XX is OBn. In another embodiment, R₄ of compound of formula XII-XX is OH. In another embodiment, R₄ of compound of formula XII-XX is CF₃.

In one embodiment, R₂ of compound of formula XII, XIII, XIV, XIVa, XVII, XIX, XXI or XXIa is hydrogen; R₁ is OCH₃ and m is 3. In another embodiment, R₂ of compound of formula XII, XIII, XIV, XIVa, XVII, XIX, XXI or XXIa is hydrogen; m is 1 and R₁ is in the para position. In another embodiment, R₂ of compound of formula XII, XIII, XIV, XIVa, XVII, XIX, XXI or XXIa is hydrogen; m is 1 and R₁ is I. In another embodiment, R₂ of compound of formula XII, XIII, XIV, XIVa, XVII, XIX, XXI or XXIa is hydrogen; m is 1 and R₁ is Br. In another embodiment, R₂ of compound of formula XII, XIII, XIV, XIVa, XVII, XIX, XXI or XXIa is hydrogen; m is 1 and R₁ is F. In another embodiment, R₂ of compound of formula XII, XIII, XIV, XVa, XVII, XIX, XXI or XXIa is hydrogen; m is 1 and R₁ is Cl. In another embodiment, R₁ of compound of formula XII, XIII, XIV, XVa, XVII, XIX, XXI or XXIa is I. In another embodiment, R₁ of compound of formula XII, XIII, XIV, XIVa, XVII, XIX, XXI or XXIa is Br. In another embodiment, R₁ of compound of formula XII, XIII, XIV, XIVa, XVII, XIX, XXI or XXIa is Cl. In another embodiment, R₁ of compound of formula XII, XIII, XIV, XIVa, XVII, XIX, XXI or XXIa is F.

In one embodiment Q of compound of formula XII is H and P is

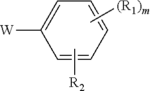

wherein W is C═O. Non-limiting examples of compounds of formula XII-XVII and XX-XXII are selected from (2-phenyl-1H-imidazol-4-yl)(3,4,5-trimethoxyphenyl)methanone (l2aa); (4-methoxyphenyl)(2-phenyl-1H-imidazol-4-yl)methanone (12ab); (3-methoxyphenyl)(2-phenyl-1H-imidazol-4-yl)methanone (12ac); (3,5-dimethoxyphenyl)(2-phenyl-1H-imidazol-4-yl)methanone (12ad); (3,4-dimethoxyphenyl)(2-phenyl-1H-imidazol-4-yl)methanone (12ae); (4-fluorophenyl)(2-phenyl-1H-imidazol-4-yl)methanonte (12af); (3-fluorophenyl)(2-phenyl-1H-imidazol-4-yl)methanone (12ag); (2-phenyl-1H-imidazol-4-yl)(p-tolyl)methanone (l2ah); (2-phenyl-1H-imidazol-4-yl)(m-tolyl)methanone (12ai); (2-(4-fluorophenyl)-1H-imidazol-4-yl)(3,4,5-trimethoxyphenyl)methanone (12ba); (2-(4-methoxyphenyl)-1H-imidazol-4-yl)(3,4,5-trimethoxyphenyl)methanone (12ca); (4-fluorphenyl)(2-(4-methoxyphenyl)-1H-imidazol-4-yl)methanone (12cb); (2-(p-tolyl)-1H-imidazol-4-yl)(3,4,5-trimethoxyphenyl)methanone (12da); (4-fluorophenyl)(2-(p-tolyl)-1H-imidazol-4-yl)methanone (12db); (4-fluorophenyl)(2-(p-tolyl)-1H-imidazol-4-yl)methanone hydrochloride (12db-Hd); (4-hydroxy-3,5-dimethoxyphenyl)(2-(p-tolyl)-1H-imidazol-4-yl)methanone (12dc); (3,4,5-trimethoxyphenyl)(2-(3,4,5-trimethoxyphenyl)-1H-imidazol-4-yl)methanone (12ea); (4-fluorophenyl)(2-(3,4,5-trimethoxyphenyl)-1H-imidazol-4-yl)methanone (12eb); (2-(4-chlorophenyl)-1H-imidazol-4-yl)(3,4,5-trimethoxyphenyl)methanone (12fa); (2-(4-chlorophenyl)-1H-imidazol-4-yl)(4-fluorophenyl)methanone (12fb); (2-(4-chlorophenyl)-1H-imidazol-4-yl)(4-hydroxy-3,5-dimethoxyphenyl)methanone (12fc); (2-(4-(dimethylamino)phenyl)-1H-imidazol-4-yl)(3,4,5-trimethoxyphenyl)methanone (12ga); (2-(4-(dimethylamino)phenyl)-1H-imidazol-4-yl)(4-fluorophenyl)methanone (12gb); (2-(3,4-dimethoxyphenyl)-1H-imidazol-4-yl)(3,4,5-trimethoxyphenyl)methanone (12ha); (2-(3,4-dimethoxyphenyl)-1H-imidazol-4-yl)(4-fluorophenyl)methanone (12hb); (2-(2-(trifluoromethyl)phenyl)-1H-imidazol-4-yl)(3,4,5-trimethoxyphenyl)methanone (121a); (4-fluorophenyl)(2-(2-(trifluoromethyl)phenyl)-1H-imidazol-4-yl)methanone (12ib); (2-(4-(benzyloxy)phenyl)-1H-imidazol-4-yl)(3,4,5-trimethoxyphenyl)methanone (121a); (2-(4-(benzyloxy)phenyl)-1H-imidazol-4-yl)(4-fluorophenyl)methanone (12jb); (2-(4-hydroxyphenyl)-1H-imidazol-4-yl)(3,4,5-trimethoxyphenyl)methanone (12ka); (2-(4-(hydroxyphenyl)-1H-imidazol-4-yl)(4-fluorophenyl)methanone (12kb); (2-(4-bromophenyl)-1H-imidazol-4-yl)(3,4,5-trimethoxyphenyl)methanone (12la); (2-(4-(trifluoromethyl)phenyl)-1H-imidazol-4-yl)(3,4,5-trimethoxyphenyl)methanone (12pa); (3,4,5-trihydroxyphenyl)(2-(3,4,5-trihydroxyphenyl)-1H-imidazol-4-yl)methanone (13ea); (2-(4-chlorophenyl)-1H-imidazol-4-yl)(3,4,5-trihydroxyphenyl)methanone (l3fa); and 2-(3,4-dihydroxyphenyl)-1H-imidazol-4-yl)(3,4,5-trihydroxyphenyl)methanone (l3ha).

In one embodiment, P of compound of formula XII is

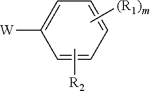

and Q is SO₂-Ph. Non-limiting examples of compound of formula XII wherein P of compound of formula XII is

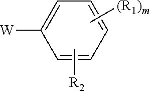

and Q is SO₂-Ph are selected from (4-methoxyphenyl)(2-phenyl-1-(phenylsulfonyl)-1H-imidazol-4-yl)methanone (11ab); (3-methoxyphenyl)(2-phenyl-1-(phenylsulfonyl)-1H-imidazol-4-yl)methanone (11ac); (2-phenyl-1-(phenylsulfonyl)-1H-imidazol-4-yl)(p-tolyl)methanone (11ah); (4-fluorophenyl)(2-phenyl-1-(phenylsulfonyl)-1H-imidazol-4-yl)methanone (11af); (3-fluorphenyl)(2-phenyl-1-(phenylsulfonyl)-1H-imidazol-4-yl)methanone (11ag); (4-fluorophenyl)(2-(4-methoxyphenyl)-1-(phenylsulfonyl)-1H-imidazol-4-yl)methanone (11cb); (1-(phenylsulfonyl)-2-(p-tolyl)-1H-imidazol-4-yl)(3,4,5-trimethoxyphenyl)methanone (11da); (4-fluorophenyl)(1-(phenylsulfonyl)-2-(p-tolyl)-1H-imidazol-4-yl)methanone (11db); (1-(phenylsulfonyl)-2-(3,4,5-trimethoxyphenyl)-1H-imidazol-4-yl)(3,4,5-trimethoxyphenyl)methanone (11ea); (4-fluorophenyl)(1-(phenylsulfonyl)-2-(3,4,5-trimethoxyphenyl)-1H-imidazol-4-yl)methanone (11eb); (2-(4-chlorophenyl)-1-(phenylsulfonyl)-1H-imidazol-4-yl)(4-fluorophenyl)methanone (11fb); (2-(4-(dimethylamino)phenyl)-1-(phenylsulfonyl)-1H-imidazol-4-yl)(3,4,5-trimethoxyphenyl)methanone (11ga); (2-(4-(dimethylamino)phenyl)-1-(phenylsulfonyl)-1H-imidazol-4-yl)(4-fluorophenyl)methanone (11gb); (2-(3,4-dimethoxyphenyl)-1-(phenylsulfonyl)-1H-imidazol-4-yl)(3,4,5-trimethoxyphenyl)methanone (11ha); (2-(3,4-dimethoxyphenyl)-1-(phenylsulfonyl)-1H-imidazol-4-yl)(4-fluorophenyl)methanone (11hb); (1-(phenylsulfonyl)-2-(2-(trifluoromethyl)phenyl)-1H-imidazol-4-yl)(3,4,5-trimethoxyphenyl)methanone (11ia); (1-(phenylsulfonyl)-2-(2-(trifluoromethyl)phenyl)-1H-imidazol-4-yl)(4-fluorophenyl)methanone (11ib); and (2-(4-(benzyloxy)phenyl)-1-(phenylsulfonyl)-1H-imidazol-4-yl)(4-fluorophenyl)methanone (11jb); (2-(4-bromophenyl)-1-(phenylsulfonyl)-1H-imidazol-4-yl)(3,4,5-trimethoxyphenyl)methanone (11la); (1-(phenysulfonyl)-2-(4-(trifluoromethyl)phenyl)-1H-imidazol-4-yl)(3,4,5-trimethoxyphenyl)methanone (11pa).

In one embodiment, R₄ and R₅ of compounds of formula XIII-XVI are hydrogens. Non-limiting examples of compounds of formula XIII-XVI wherein R₄ and R₅ are hydrogens are selected from (2-phenyl-1H-imidazol-4-yl)(3,4,5-trimethoxyphenyl)methanone (12aa); (4-methoxyphenyl)(2-phenyl-1H-imidazol-4-yl)methanone (12ab); (3-methoxyphenyl)(2-phenyl-1H-imidazol-4-yl)methanone (12ac); (3,5-dimethoxyphenyl)(2-phenyl-1H-imidazol-4-yl)methanone (12ad); (3,4-dimethoxyphenyl)(2-phenyl-1H-imidazol-4-yl)methanone (12ae); (4-fluorophenyl)(2-phenyl-1H-imidazol-4-yl)methanone (12af); (3-fluorophenyl)(2-phenyl-1H-imidazol-4-yl)methanone (12ag); (2-phenyl-1H-imidazol-4-yl)(p-tolyl)methanone (12ah); and (2-phenyl-1H-imidazol-4-yl)(m-tolyl)methanone (12ai).

In one embodiment, P of compound of formula XII is H and Q is

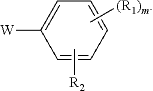

In another embodiment W is C═O. In another embodiment, W of compound of formula XVIII is C═O. Non-limiting examples of compound of formula XVIII wherein W is C═O are selected from (4-methoxyphenyl)(2-phenyl-1H-imidazol-1-yl)methanone (12aba) and (2-phenyl-1H-imidazol-1-yl)(3,4,5-trimethoxyphenyl)methanone (12aaa).

In another embodiment, W of compound of formula XVIII is SO₂. Non-limiting examples of compound of formula XVIII wherein W is SO₂ are selected from 2-phenyl-1-(phenylsulfonyl)-1H-imidazole (10a); 2-(4-nitrophenyl)-1-(phenylsulfonyl)-1H-imidazole (10x) and 2-(4-(benzyloxy)phenyl)-1-(phenylsulfonyl)-1H-imidazole (10j).

As used herein, “single-, fused- or multiple-ring, aryl or (hetero)cyclic ring systems” can be any such ring, including but not limited to phenyl, biphenyl, triphenyl, naphthyl, cycloalkyl, cycloalkenyl, cyclodienyl, fluorene, adamantane, etc.

As used herein, the term “N-heterocycles” can be any such N-containing heterocycle, including but not limited to aza- and diaza-cycloalkyls such as aziridinyl, azetidinyl, diazatidinyl, pyrrolidinyl, piperidinyl, piperazinyl, and azocanyl, pyrrolyl, pyrazolyl, imidazolyl, pyridinyl, pyrimidinyl, pyrazinyl, pyridazinyl, triazinyl, tetrazinyl, pyrrolizinyl, indolyl, quinolinyl, isoquinolinyl, benzimidazolyl, indazolyl, quinolizinyl, cinnolinyl, quinololinyl, phthalazinyl, naphthyridinyl, quinoxalinyl, etc.

As used herein, the term “O-Heterocycles” can be any such O-containing heterocycle including but not limited to oxiranyl, oxetanyl, tetrahydrofuranyl, tetrahydropyranyl, dioxanyl, furanyl, pyrylium, benzofuranyl, benzodioxolyl, etc.

As used herein, the term “S-heterocycles” can be any such S-containing heterocycle, including but not limited to thiranyl, thietanyl, tetrahydrothiophene-yl, dithiolanyl, tetrahydrothiopyranyl, thiophene-yl, thiepinyl, thianaphthenyl, etc.

As used herein, the term “Mixed heterocycles” can be any heterocycle containing two or more S—, N—, or O-heteroatoms, including but not limited to oxathiolanyl, morpholinyl, thioxanyl, thiazolyl, isothiazolyl, thiadiazolyl, oxazolyl, isoxazolyl, oxadiaziolyl, etc.

As used herein, “aliphatic straight- or branched-chain hydrocarbon” refers to both alkylene groups that contain a single carbon and up to a defined upper limit, as well as alkenyl groups and alkynyl groups that contain two carbons up to the upper limit, whether the carbons are present in a single chain or a branched chain. Unless specifically identified, a hydrocarbon can include up to about 30 carbons, or up to about 20 hydrocarbons, or up to about 10 hydrocarbons. Alkenyl and alkynyl groups can be mono-unsaturated or polyunsaturated. In another embodiment, an alkyl includes C₁-C₆ carbons. In another embodiment, an alkyl includes C₁-C₅ carbons. In another embodiment, an alkyl includes C₁-C₁₀ carbons. In another embodiment, an alkyl is a C₁-C₁₂ carbons. In another embodiment, an alkyl is a C₁-C₅ carbons.

As used herein, the term “alkyl” can be any straight- or branched-chain alkyl group containing up to about 30 carbons unless otherwise specified. In another embodiment, an alkyl includes C₁-C₆ carbons. In another embodiment, an alkyl includes C₁-C₅ carbons. In another embodiment, an alkyl includes C₁-C₁₀ carbons. In another embodiment, an alkyl is a C₁-C₁₂ carbons. In another embodiment, an alkyl is a C₁-C₂₀ carbons. In another embodiment, cyclic alkyl group has 3-8 carbons. In another embodiment, branched alkyl is an alkyl substituted by alkyl side chains of 1 to 5 carbons.

The alkyl group can be a sole substituent or it can be a component of a larger substituent, such as in an alkoxy, haloalkyl, arylalkyl, alkylamino, dialkylamino, alkylamido, alkylurea, etc. Preferred alkyl groups are methyl, ethyl, and propyl, and thus halomethyl, dihalomethyl, trihalomethyl, haloethyl, dihaloethyl, trihaloethyl, halopropyl, dihalopropyl, trihalopropyl, methoxy, ethoxy, propoxy, arylmethyl, arylethyl, arylpropyl, methylamino, ethylamino, propylamino, dimethylamino, diethylamino, methylamido, acetamido, propylamido, halomethylamido, haloethylamido, halopropylamido, methyl-urea, ethyl-urea, propyl-urea, etc.

As used herein, the term “aryl” refers to any aromatic ring that is directly bonded to another group. The aryl group can be a sole substituent, or the aryl group can be a component of a larger substituent, such as in an arylalkyl, arylamino, arylamido, etc. Exemplary aryl groups include, without limitation, phenyl, tolyl, xylyl, furanyl, naphthyl, pyridinyl, pyrimidinyl, pyridazinyl, pyrazinyl, triazinyl, thiazolyl, oxazolyl, isooxazolyl, pyrazolyl, imidazolyl, thiophene-yl, pyrrolyl, phenylmethyl, phenylethyl, phenylamino, phenylamido, etc.

As used herein, the term “aminoalkyl” refers to an amine group substituted by an alkyl group as defined above. Aminoalkyl refers to monoalkylamine, dialkylamine or trialkylamine. Nonlimiting examples of aminoalkyl groups are —N(Me)₂, —NHMe, —NH₃.

A “haloalkyl” group refers, in another embodiment, to an alkyl group as defined above, which is substituted by one or more halogen atoms, e.g. by F, Cl, Br or I. Nonlimiting examples of haloalkyl groups are CF₃, CF₂CF₃, CH₂CF₃.

In one embodiment, this invention provides a compound used in this invention or its isomer, metabolite, pharmaceutically acceptable salt, pharmaceutical product, tautomer, hydrate, N-oxide, polymorph, or crystal or combinations thereof. In one embodiment, this invention provides an isomer of the compound of this invention. In another embodiment, this invention provides a metabolite of the compound of this invention. In another embodiment, this invention provides a pharmaceutically acceptable salt of the compound of this invention. In another embodiment, this invention provides a pharmaceutical product of the compound of this invention. In another embodiment, this invention provides a tautomer of the compound of this invention. In another embodiment, this invention provides a hydrate of the compound of this invention. In another embodiment, this invention provides an N-oxide of the compound of this invention. In another embodiment, this invention provides a polymorph of the compound of this invention. In another embodiment, this invention provides a crystal of the compound of this invention. In another embodiment, this invention provides composition comprising a compound of this invention, as described herein, or, in another embodiment, a combination of an isomer, metabolite, pharmaceutically acceptable salt, pharmaceutical product, tautomer, hydrate, N-oxide, polymorph, or crystal of the compound of this invention.

In one embodiment, the term “isomer” includes, but is not limited to, optical isomers and analogs, structural isomers and analogs, conformational isomers and analogs, and the like.

In one embodiment, the compounds of this invention are the pure (E)-isomers. In another embodiment, the compounds of this invention are the pure (Z)-isomers. In another embodiment, the compounds of this invention are a mixture of the (E) and the (Z) isomers. In one embodiment, the compounds of this invention are the pure (R)-isomers. In another embodiment, the compounds of this invention are the pure (S)-isomers. In another embodiment, the compounds of this invention are a mixture of the (R) and the (5) isomers.

The compounds of the present invention can also be present in the form of a racemic mixture, containing substantially equivalent amounts of stereoisomers. In another embodiment, the compounds of the present invention can be prepared or otherwise isolated, using known procedures, to obtain a stereoisomer substantially free of its corresponding stereoisomer (i.e., substantially pure). By substantially pure, it is intended that a stereoisomer is at least about 95% pure, more preferably at least about 98% pure, most preferably at least about 99% pure.

Compounds of the present invention can also be in the form of a hydrate, which means that the compound further includes a stoichiometric or non-stoichiometric amount of water bound by non-covalent intermolecular forces.

Compounds of the present invention may exist in the form of one or more of the possible tautomers and depending on the particular conditions it may be possible to separate some or all of the tautomers into individual and distinct entities. It is to be understood that all of the possible tautomers, including all additional enol and keto tautomers and/or isomers are hereby covered. For example, the following tautomers, but not limited to these, are included.

Tautomerization of the Imidazole Ring

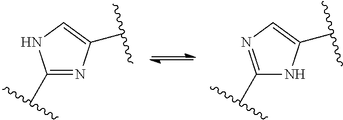 [SMILES: *c1c[nH]c(*)n1 ; *c1cnc(*)[nH]1]

The invention includes “pharmaceutically acceptable salts” of the compounds of this invention, which may be produced, by reaction of a compound of this invention with an acid or base. Certain compounds, particularly those possessing acid or basic groups, can also be in the form of a salt, preferably a pharmaceutically acceptable salt. The term “pharmaceutically acceptable salt” refers to those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxylic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

Suitable pharmaceutically-acceptable salts of amines of compounds the compounds of this invention may be prepared from an inorganic acid or from an organic acid. In one embodiment, examples of inorganic salts of amines are bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphate, sulfates, sulfamates, sulfanilates, sulfonic acids (alkylsulfonates, arylsulfonates, halogen substituted alkylsulfonates, halogen substituted arylsulfonates), sulfonates and thiocyanates.

In one embodiment, examples of organic salts of amines may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates gluconates, glutamates, glycolates, glucorate, glucoheptanoates, glycerophosphates, gluceptates, glycollylarsanilates, glutarates, glutamate, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, nitrates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartrates, theophyllineacetates, p-toluenesulfonates (tosylates), trifluoroacetates, terephthalates, tannates, teoclates, trihaloacetates, triethiodide, tricarboxylates, undecanoates and valerates.

In one embodiment, examples of inorganic salts of carboxylic acids or hydroxyls may be selected from ammonium, alkali metals to include lithium, sodium, potassium, cesium; alkaline earth metals to include calcium, magnesium, aluminium; zinc, barium, cholines, quaternary ammoniums.

In another embodiment, examples of organic salts of carboxylic acids or hydroxyl may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglamines, N-methyl-D-glucamines, N,N′-dibenzylethylenediamines, nicotinamides, organic amines, ornithines, pyridines, picolies, piperazines, procain, tris(hydroxymethyl)methylamines, triethylamines, triethanolamines, trimethylamines, tromethamines and ureas.

In one embodiment, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of an existing salt for another ion or suitable ion-exchange resin.

The compounds used in the method of the invention are synthesized according to published methods. In particular, the compounds are synthesized according to the methods described in PCT publication Nos. WO 2010/74776, published Jul. 1, 2010; WO 2011/19059, published Sep. 9, 2010; and WO 2012/027481, published Mar. 1, 2012, hereby incorporated by reference.

### Pharmaceutical Composition

Another aspect of the present invention relates to a pharmaceutical composition for use in treating pancreatic cancer including a pharmaceutically acceptable carrier and at least one compound described above. Typically, the pharmaceutical composition of the present invention will include a compound or its pharmaceutically acceptable salt, as well as a pharmaceutically acceptable carrier. The term “pharmaceutically acceptable carrier” refers to any suitable adjuvants, carriers, excipients, or stabilizers, and can be in solid or liquid form such as, tablets, capsules, powders, solutions, suspensions, or emulsions.

Typically, the composition will contain from about 0.01 to 99 percent, preferably from about 20 to 75 percent of active compound(s), together with the adjuvants, carriers and/or excipients. While individual needs may vary, determination of optimal ranges of effective amounts of each component is within the skill of the art. Typical dosages comprise about 0.01 to about 100 mg/kg body wt. The preferred dosages comprise about 0.1 to about 100 mg/kg body wt. The most preferred dosages comprise about 1 to about 100 mg/kg body wt. Treatment regimen for the administration of the compounds of the present invention can also be determined readily by those with ordinary skill in art. That is, the frequency of administration and size of the dose can be established by routine optimization, preferably while minimizing any side effects.

The solid unit dosage forms can be of the conventional type. The solid form can be a capsule and the like, such as an ordinary gelatin type containing the compounds and a carrier, for example, lubricants and inert fillers such as, lactose, sucrose, or cornstarch. The compounds may be tabulated with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The tablets, capsules, and the like can also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it can contain, in addition to materials of the above type, a liquid carrier such as a fatty oil.

Various other materials may be present as coatings or to modify the physical form of the dosage unit. For instance, tablets can be coated with shellac, sugar, or both. A syrup can contain, in addition to active ingredient, sucrose as a sweetening agent, methyl and propylparabens as preservatives, a dye, and flavoring such as cherry or orange flavor.

For oral therapeutic administration, the active compounds can be incorporated with excipients and used in the form of tablets, capsules, elixirs, suspensions, syrups, and the like. Such compositions and preparations should contain at least 0.1% of active compound. The percentage of the compound in these compositions can, of course, be varied and can conveniently be between about 2% to about 60% of the weight of the unit. The amount of active compound in such therapeutically useful compositions is such that a suitable dosage will be obtained. Preferred compositions according to the present invention are prepared so that an oral dosage unit contains between about 1 mg and 800 mg of active compound.

The active compounds or formulations thereof may be orally administered, for example, with an inert diluent, or with an assimilable edible carrier, or they can be enclosed in hard or soft shell capsules, or they can be compressed into tablets, or they can be incorporated directly with the food of the diet.

The pharmaceutical forms suitable for injectable use include sterile aqueous solutions or dispersions and sterile powders for the extemporaneous preparation of sterile injectable solutions or dispersions. In all cases, the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The compounds or pharmaceutical compositions of the present invention may also be administered in injectable dosages by solution or suspension of these materials in a physiologically acceptable diluent with a pharmaceutical adjuvant, carrier or excipient. Such adjuvants, carriers and/or excipients include, but are not limited to, sterile liquids, such as water and oils, with or without the addition of a surfactant and other pharmaceutically and physiologically acceptable components. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

The active compounds or formulations thereof may also be administered parenterally. Solutions or suspensions of these active compounds can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

For use as aerosols, the compounds or formulations thereof in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The compounds used in the methods of the invention are administered in combination with an anti-cancer agent. In one embodiment, the anti-cancer agent is a monoclonal antibody. In some embodiments, the monoclonal antibodies are used for diagnosis, monitoring, or treatment of cancer. In one embodiment, monoclonal antibodies react against specific antigens on cancer cells. In one embodiment, the monoclonal antibody acts as a cancer cell receptor antagonist. In one embodiment, monoclonal antibodies enhance the patient's immune response. In one embodiment, monoclonal antibodies act against cell growth factors, thus blocking cancer cell growth. In one embodiment, anti-cancer monoclonal antibodies are conjugated or linked to anti-cancer drugs, radioisotopes, other biologic response modifiers, other toxins, or a combination thereof. In one embodiment, anti-cancer monoclonal antibodies are conjugated or linked to a compound as described hereinabove.

Yet another aspect of the present invention relates to a method of treating pancreatic cancer that includes selecting a subject in need of treatment the cancer and administering to the subject a pharmaceutical composition comprising at least one compound and a pharmaceutically acceptable carrier under conditions effective to treat the cancer. The method may include a pharmaceutical composition containing at least one additional compound for the treatment of pancreatic cancer.

When administering the compounds, they can be administered systemically or, alternatively, they can be administered directly to a specific site where cancer cells or precancerous cells are present. Thus, administering can be accomplished in any manner effective for delivering the compounds or the pharmaceutical compositions to the cancer cells or precancerous cells. Exemplary modes of administration include, without limitation, administering the compounds or compositions orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes.

### Biologic Activity

The invention encompasses compounds and compositions for use in treating pancreatic cancer. The compositions may further comprise additional active ingredients, whose activity is useful for the treatment of pancreatic cancer.

Drug resistance is the major cause of cancer chemotherapy failure. One major contributor to multidrug resistance is overexpression of P-glycoprotein (P-gp). This protein is a clinically important transporter protein belonging to the ATP-binding cassette family of cell membrane transporters. It can pump substrates including anticancer drugs out of tumor cells through an ATP-dependent mechanism.

The management of pancreatic cancer (PanCa) is exceptionally difficult due to poor response to available therapeutic drugs. Tubulins play a major role in cell dynamics and are important molecular targets for cancer therapy. Among various tubulins, βIII and βIV-tubulin isoforms have been primarily implicated in Pancreatic cancer progression, metastasis and chemo-resistance. However, specific inhibitors of these isoforms that have potent anti-cancer activity with low toxicity are not readily available. The molecules of the invention preferentially repressed βIII and βIV tubulin isoforms via restoring the expression of miR-200c that directly target these isoforms. As a result, the molecules of the invention efficiently inhibited tumorigenic and metastatic characteristics of pancreatic cancer cells in nanomolar range concentrations.

The ABI molecules, discussed herein, arrested the cell cycle in the G2/M phase and induced apoptosis in pancreatic cancer cell lines via modulation of cell cycle regulatory (Cdc2, Cdc25c, and Cyclin B1) and apoptosis associated (Bax, Bad, Bcl-2, and Bcl-xl) proteins. As illustrates in the examples, the treatment effectively inhibited pancreatic tumor growth in preclinical xenograft mouse model.

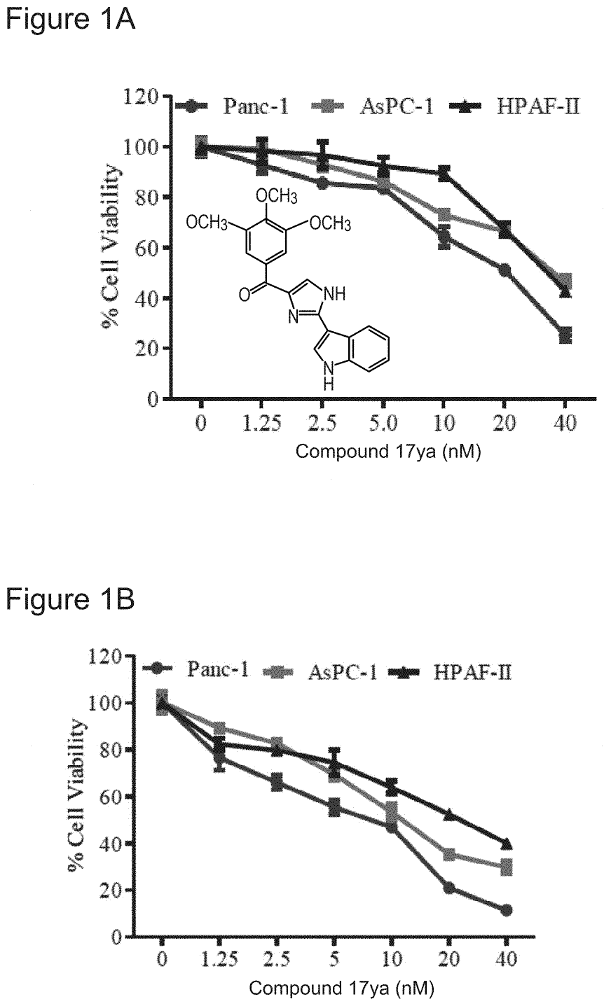

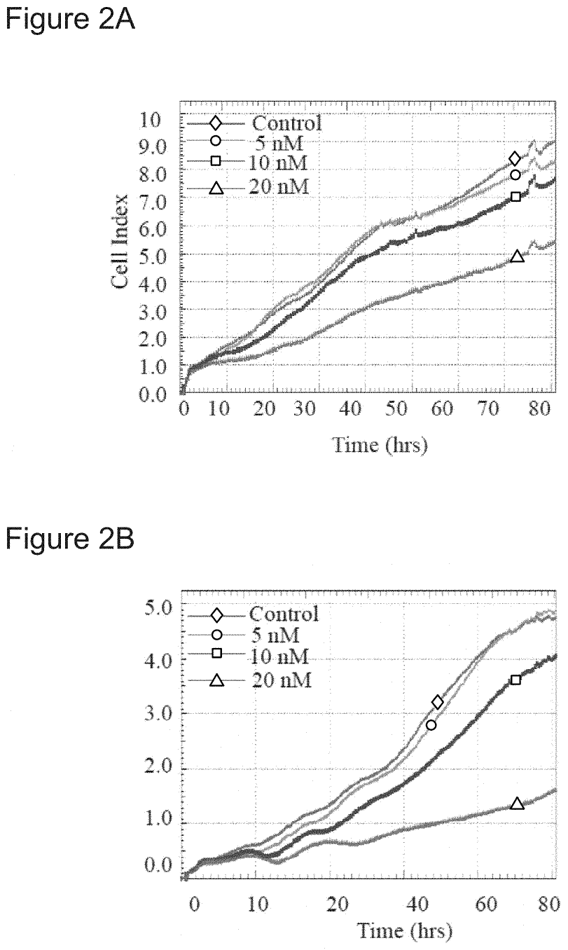

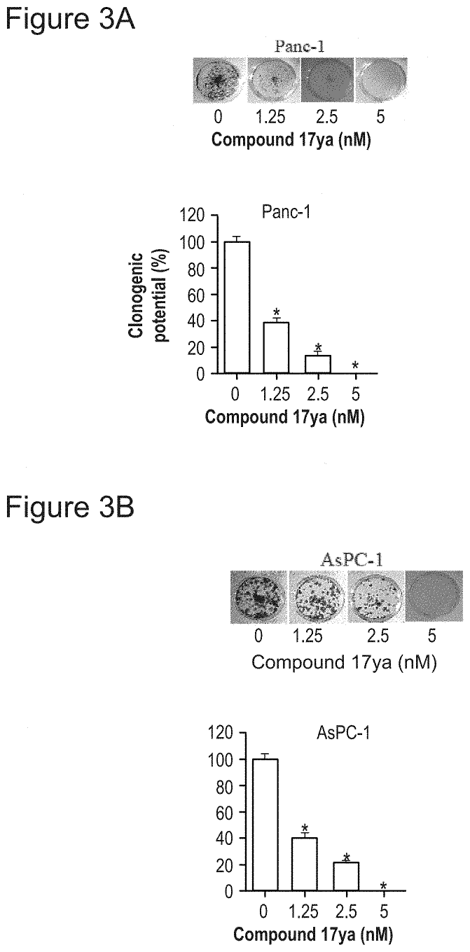

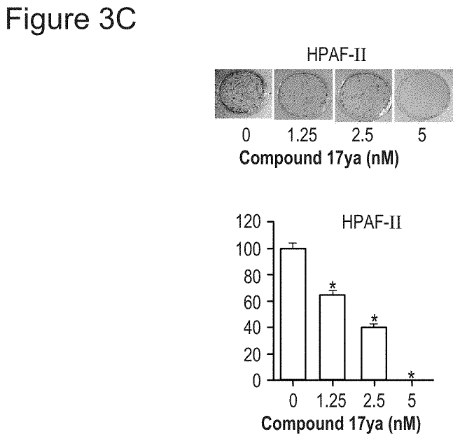

The cytotoxic effect of Compound 17ya against various human pancreatic cancer cell lines was studied, the cell lines included AsPC-1, Panc-1 and HPAF-II. The cells were treated with various concentrations of Compound 17ya (1.25-160 nM) for 24 and 48 hrs., and cell viability was determined by MTT assay. The compound inhibited the growth of pancreatic cancer cells in a dose- and time-dependent manner. The results are illustrated in FIG. 1A. The IC₅₀ for Compound 17ya was 20, 30 and 30 nM in Panc-1, AsPC-1 and HPAF-II, respectively, after 24 hrs. treatment. The IC₅₀ after 48 hrs post-treatment was 8.2, 12.5, and 20 nM, respectively. The results of this study are shown in FIG. 1B. Further, the growth inhibitory effect of Compound 17ya was determined in real time with the xCELLigence system. The growth curve, recorded as the basal cell index value, showed that Compound 17ya significantly reduced the cell index in a dose-dependent manner compared to vehicle treated pancreatic cancer cells. The results are illustrated in FIG. 2A (Panc-1) and FIG. 2B (AsPC-1). Colony formation assays in pancreatic cancer cells demonstrated that Compound 17ya (1.25-5 nM) significantly reduced the number of colonies in a dose-dependent manner in Panc-1 (FIG. 3A), AsPC-1 (FIG. 3B) and HPAF-II (FIG. 3C) cells as compared to control groups.

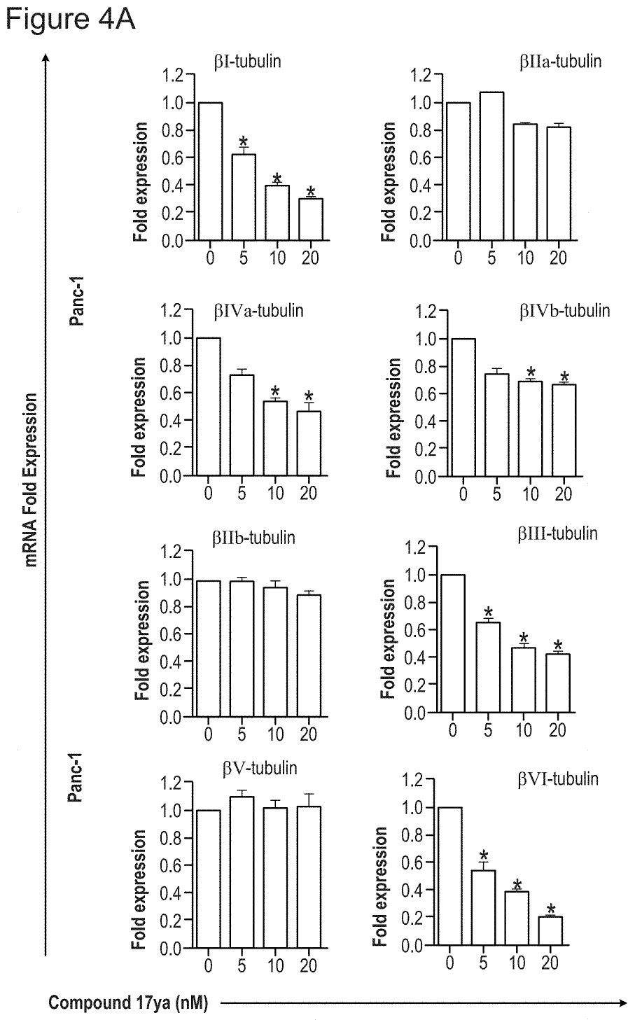

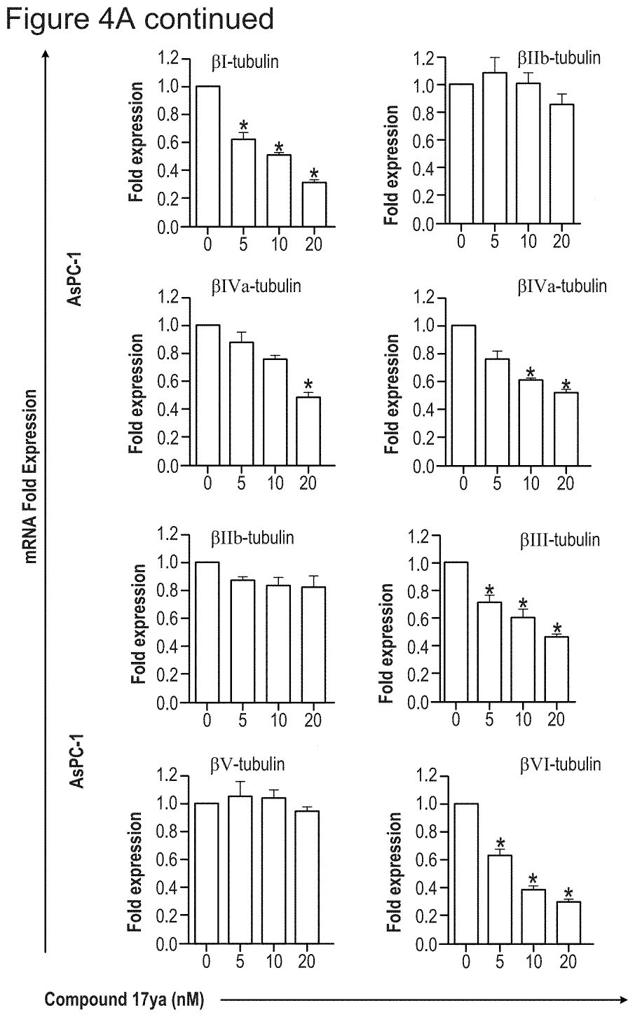

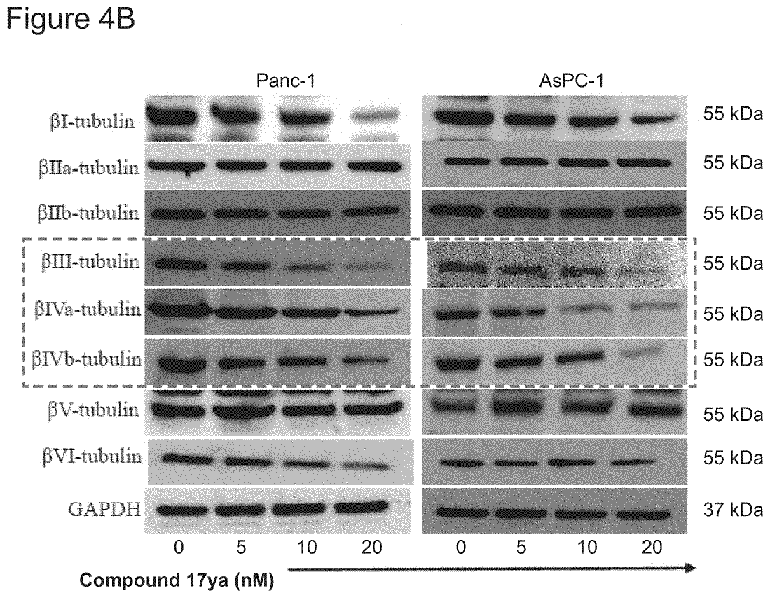

Compound 17ya inhibited mRNA expression and protein stability of β-tubulin isotypes in pancreatic cancer cells. The effect of Compound 17ya on the expression of βIII and βIV-tubulins in pancreatic cancer cells was determined at doses of 5-20 nM and treatment significantly (p<0.01) inhibited the mRNA expression of βIII and βIV-tubulins, in a dose-dependent manner in both Panc-1 (FIG. 4A) and AsPC-1 cells (FIG. 4A) as determined by qRT-PCR.

Western blot analysis demonstrated that Compound 17ya inhibited protein levels of βIII and βIV-tubulins in both Panc-1 and AsPC-1 cells. Compound 17ya inhibited the mRNA and protein expression of βI-tubulins in Panc-1 and AsPC-1 cells as illustrated in FIGS. 4A and 4B. However, no effect was observed on either mRNA or protein for βIIa, βIIb and βV-tubulins in any tested pancreatic cancer cells.

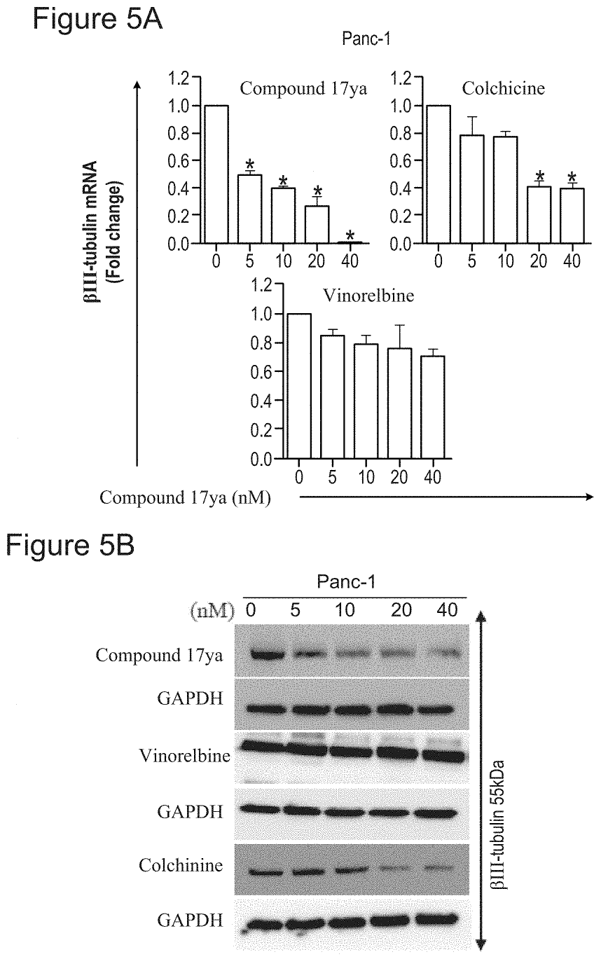

The effect of Compound 17ya on the expression of 0111-tubulin was compared with known β-tubulin-destabilizers colchicine, and vinorelbine. Panc-1 cells were treated with 5-40 nM of Compound 17ya, colchicine, or vinorelbine for 24 hrs. RNA and protein lysates were prepared to determine the mRNA expression and protein level of 0111-tubulin. The results of tests are illustrated in FIG. 5A. In comparison to colchicine, Compound 17ya more effectively inhibited mRNA expression (FIG. 5A) and protein levels (FIG. 5B) of βIII-tubulin.

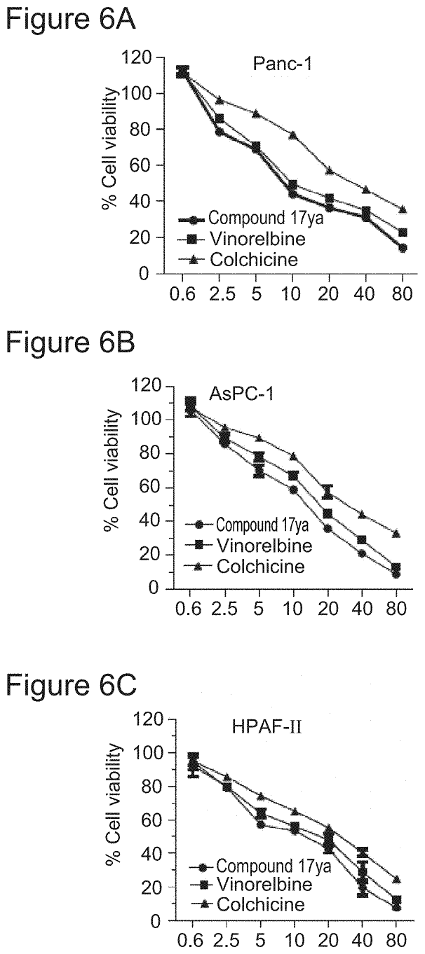

Cell proliferation was determined by MTT assays. Compound 17ya was compared with colchicine and vinorelbine in Panc-1, AsPC-1, and HPAF-II cells. Compound 17ya was the most effective cell growth inhibitor in all pancreatic cancer cell lines as compared to colchicine and vinorelbine. The results are illustrated in FIG. 6A.

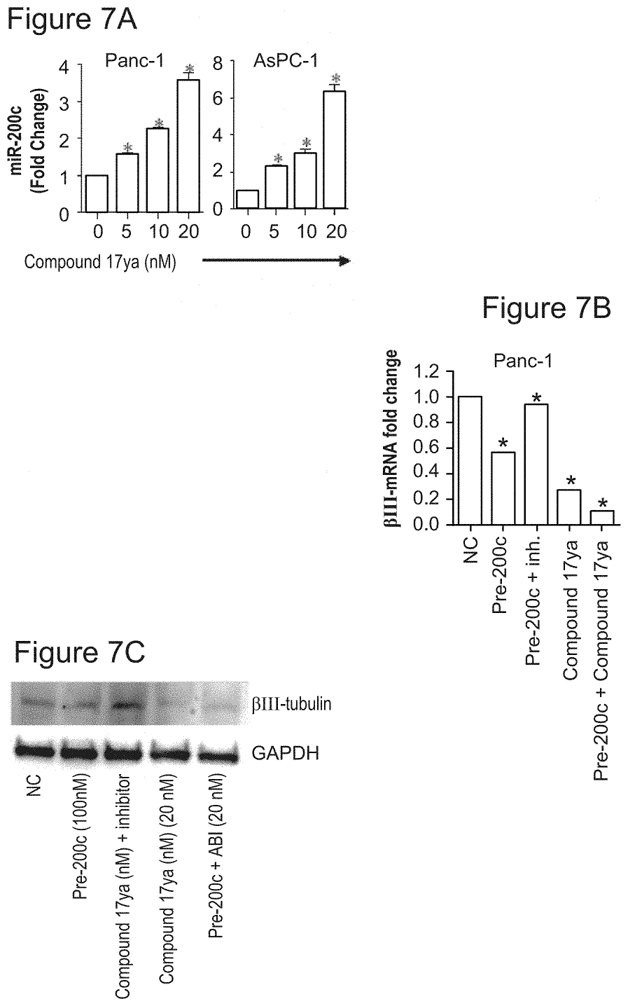

Compound 17ya was tested to determine whether the compound inhibited 111-tubulin expression via targeting miR-200c. Compound 17ya induced the expression of miR-200c in Panc-1 and AsPC-1 when compared with control cells. The results are illustrated in FIGS. 7A and 7B. Transfection of miR-200c mimics in Panc-1 cells inhibited the expression of βIII-tubulin, which was rescued by transfecting the cells with miR-200c inhibitor. This is illustrated in FIG. 7B. Treatment of Compound 17ya and miR-200c mimic transfection of Panc-1 cells showed a synergistic effect on the expression of βIII tubulin at both mRNA and protein levels as illustrated in FIGS. 7B and 7C, respectively. The results demonstrated that Compound 17ya inhibited the expression of βIII tubulin via restoring the expression of miR-200c in pancreatic cancer cells.

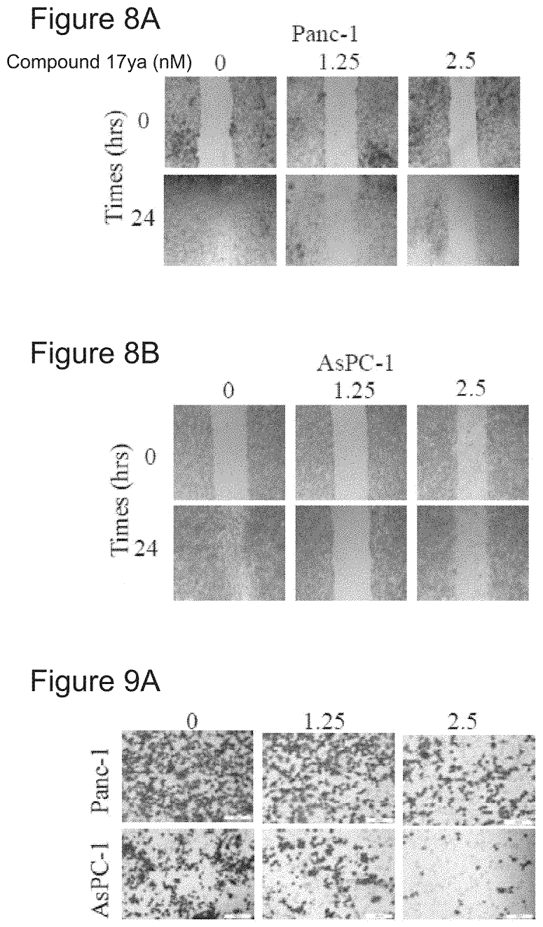

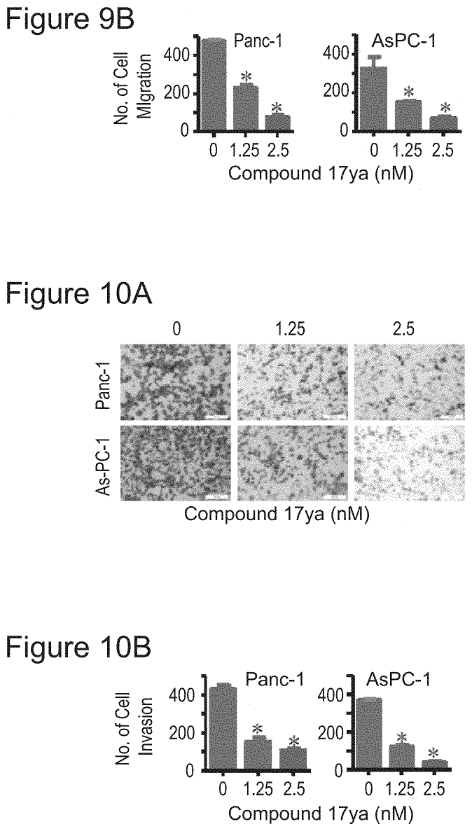

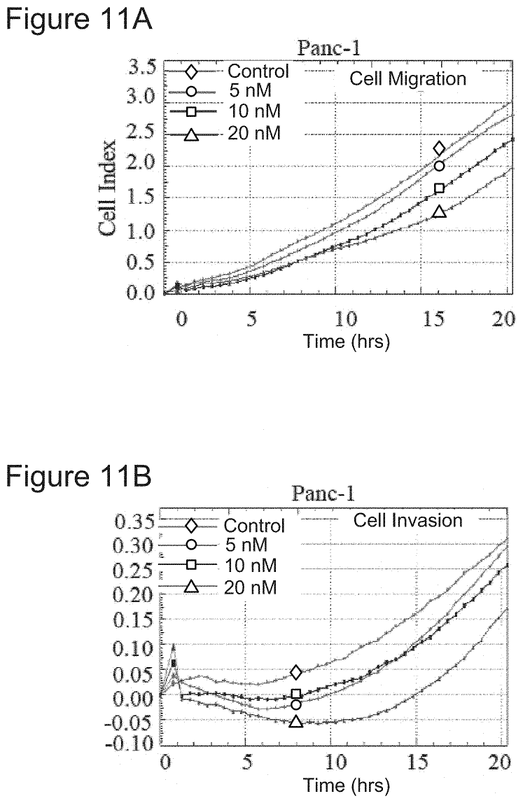

Wound healing assays determined the effect of Compound 17ya on the migration of pancreatic cancer cells. Remarkable inhibition in migration of both Panc-1, AsPC-1, and HPAF-II cells was obtained when treated with sub-lethal concentrations of Compound 17ya (1.25 and 2.5 nM). The results are illustrated in FIGS. 8A and 8B. Compound 17ya was tested at 0, 1.25, and 2.5 nM with Panc-1 and AsPC-1. The results showed significant (p<0.01) inhibition of Panc-1, AsPC-1, and HPAF-II cell migration in a dose-dependent manner. The results are illustrated in FIG. 9A and FIG. 9B. At sub lethal concentrations (1.25-2.5 nM), Compound 17ya significantly (p<0.01) inhibited invasion of Panc-1, AsPC-1 and HPAF-II cells as compared to the vehicle treatment group as illustrated in FIGS. 10A and 10B. Compound 17ya dose-dependently (5-20 nM) reduced the baseline cell index of pancreatic cancer cells as compared to control demonstrating the potent inhibitory effect of Compound 17ya on pancreatic cancer cell migration and invasion. The results of this study are illustrated in FIGS. 11A and 11B, respectively.

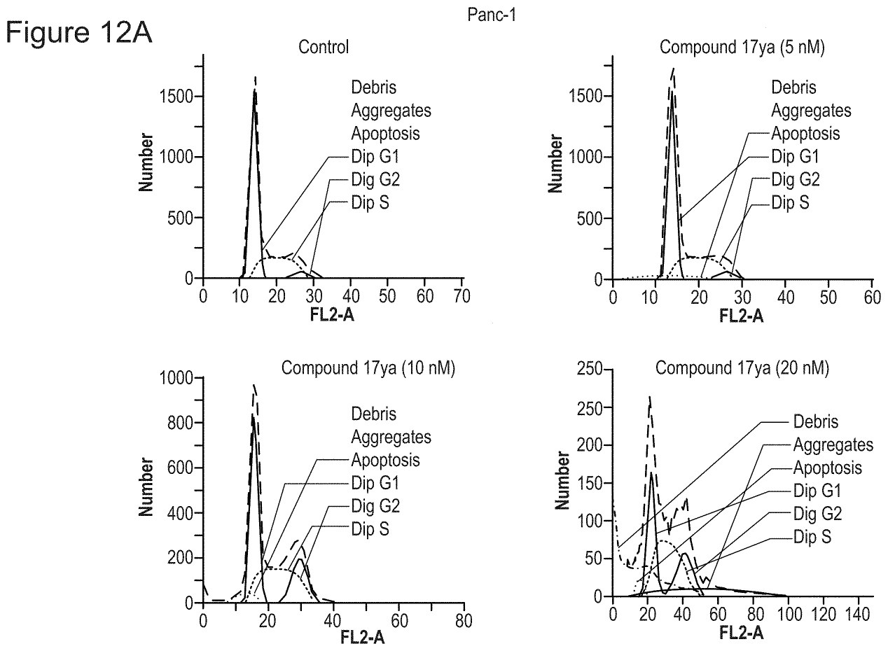

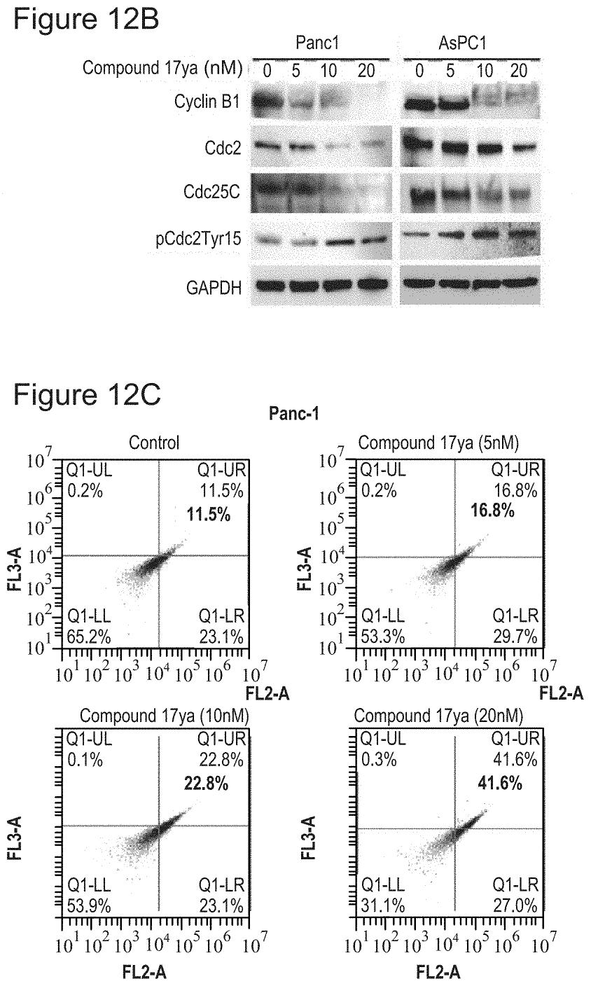

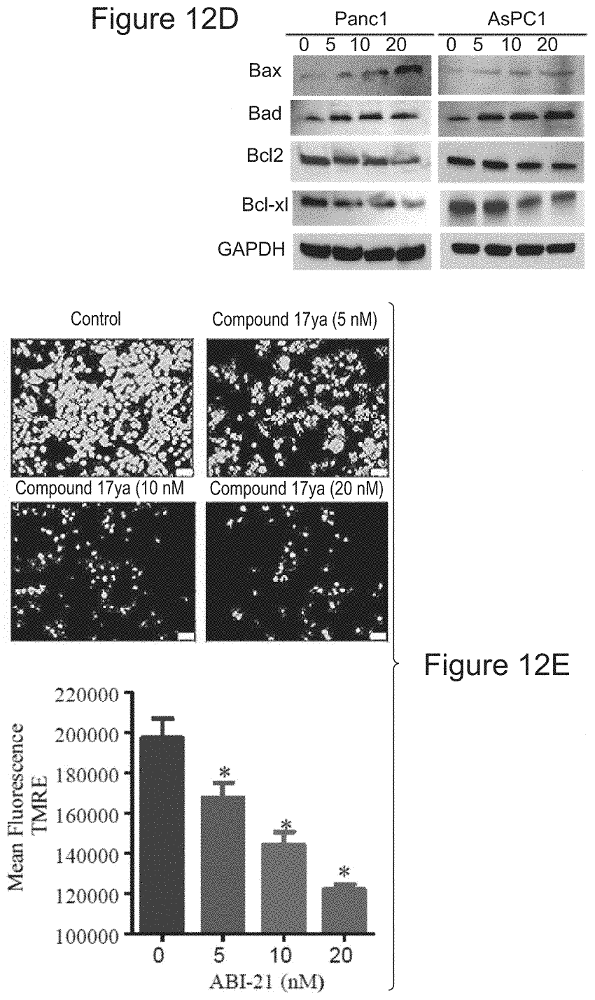

The effect of Compound 17ya on cell cycle distribution of pancreatic cancer cells was examined by flow cytometry. FIG. 12A illustrates the results of Compound 17ya treatment which arrested the cell cycle of Panc-1 and AsPC-1 cells in G2/M phase in a dose-dependent manner. A control group was compared to Compound 17ya at 10 nM, 20 nM, and 40 nM. The results are illustrated in the following table.

| Groups | G₀-G₁ | S | G₂M |
|---|---|---|---|
| Control | 61.8% | 32.0% | 4.9% |
| Compound 17ya (10 nM) | 61.0% | 33.8% | 5.1% |
| Compound 17ya (20 nM) | 46.6% | 33.6% | 19.6% |
| Compound 17ya (40 nM) | 32.5% | 45.9% | 21.5% |

As shown in FIG. 12B, Compound 17ya dose-dependently inhibited the protein levels of cyclin B1 and cdc25c in Panc-1 and AsPC-1 cells. In addition, Compound 17ya also dose-dependently (5-20 nM) inhibited both the phosphorylation and total protein of cyclin-dependent kinase Cdc2, in Panc-1 and AsPC-1 cells as shown in FIG. 12B. The effect of Compound 17ya on apoptosis induction in pancreatic cancer cells was determined by Annexin V-7AAD staining and mitochondrial membrane potential (Δψm) using flow cytometer. As shown in FIG. 12C, Compound 17ya treatment (5-20 nM) resulted in apoptosis induction in both Panc-1 and AsPC-1 cells. Compound 17ya treatment (10-20 nM) illustrated 22.8% and 41.6% apoptotic population of Panc-1 cells, whereas AsPC-1 cells showed 11.5% and 18.0% apoptotic cells respectively at same concentrations. The effect of Compound 17ya on Δψm in Panc-1 and AsPC-1 cells using TMRE staining determined a dose-dependent (5-20 nM) decrease of TMRE staining in both Panc-1 and AsPC-1 cells as illustrated in FIG. 12E. Compound 17ya (5-20 nM) induced the expression of Bax and Bad, and inhibited expression of Bcl-2 and Bcl-xl proteins. The results are illustrated in FIG. 12D. These results suggest that VERU-111 arrests the cell cycle in the G2/M phase and induces apoptosis via intrinsic mechanism in pancreatic cancer cells.

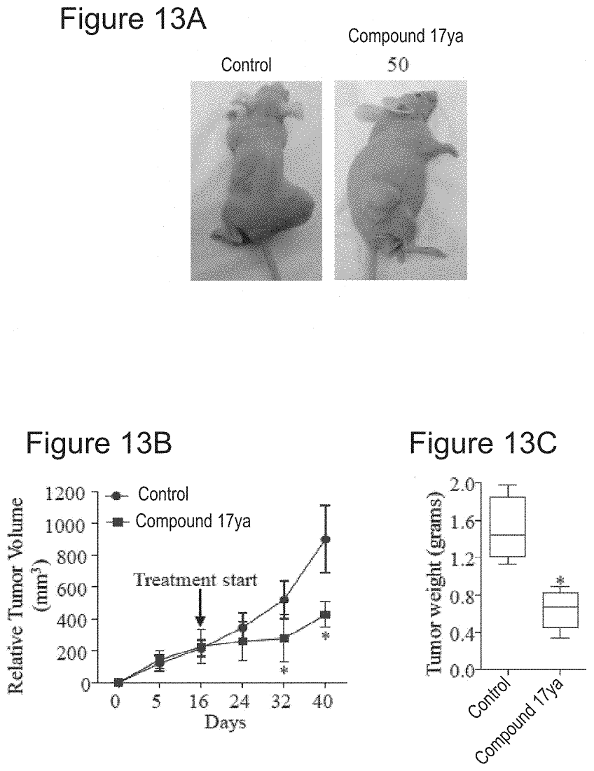

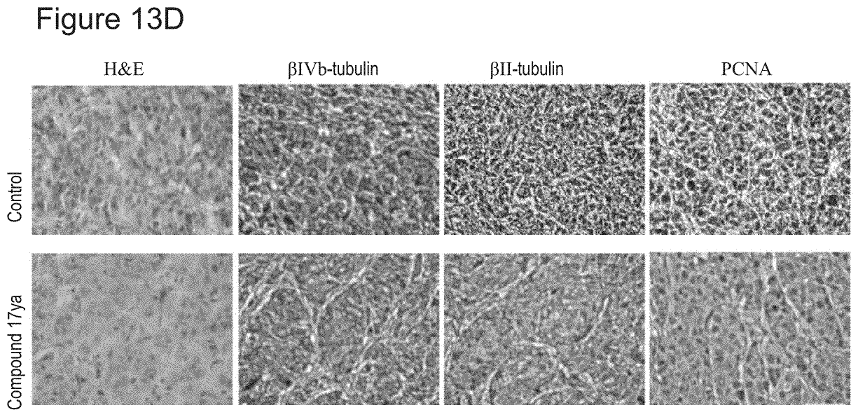

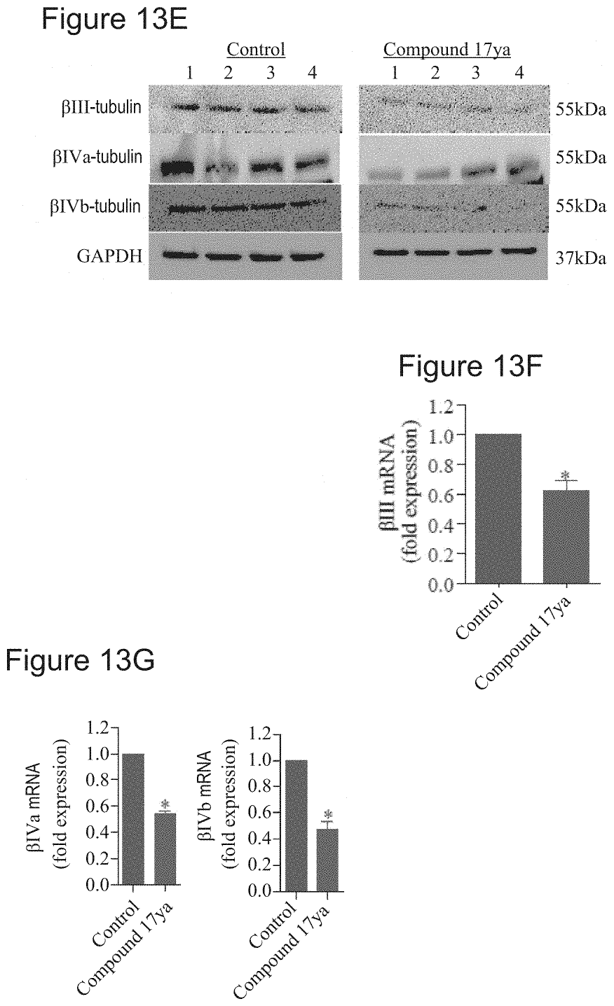

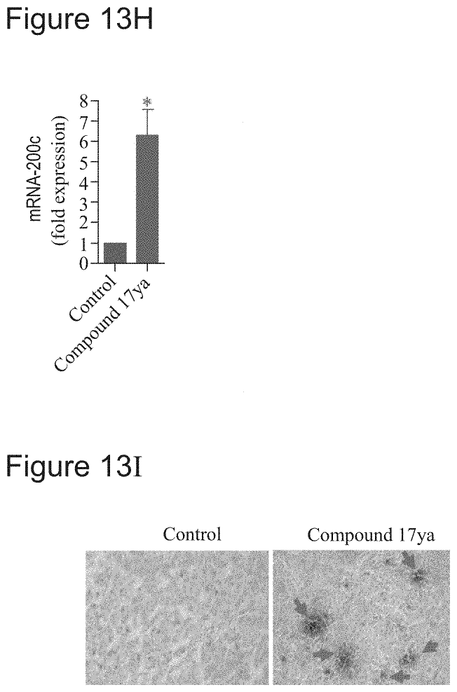

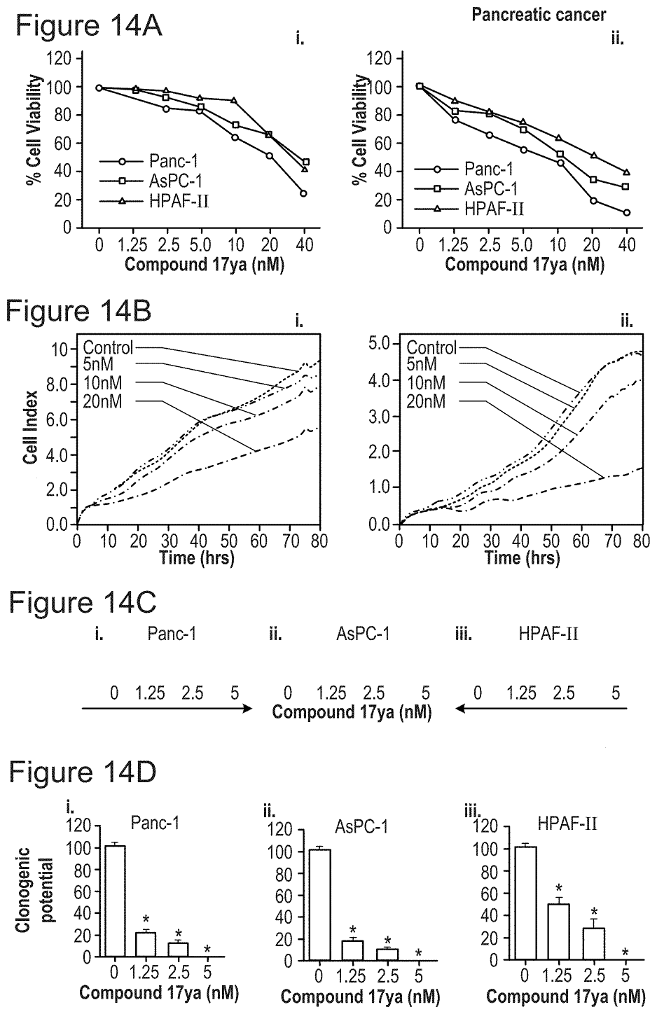

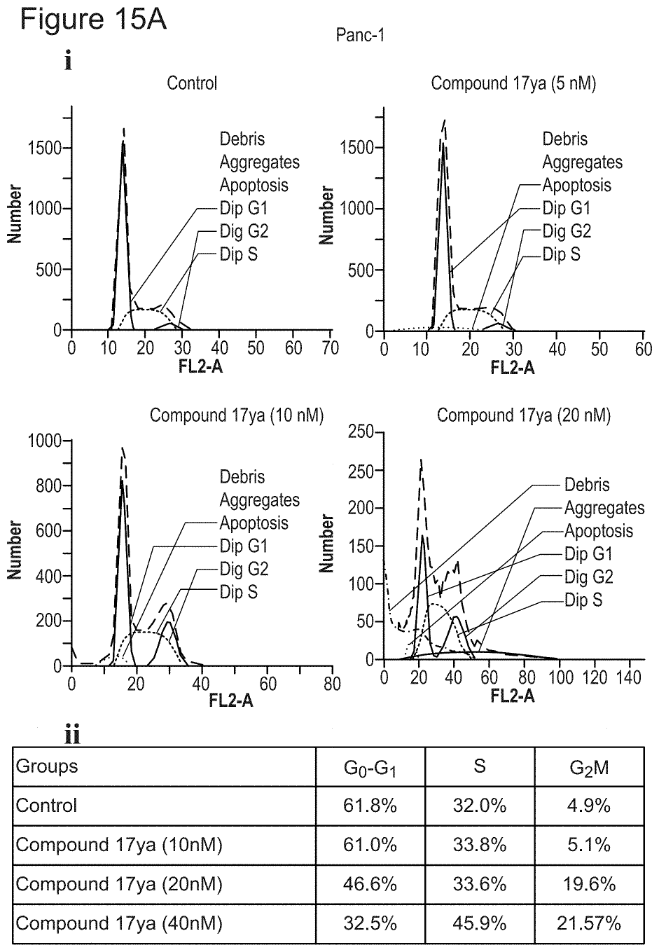

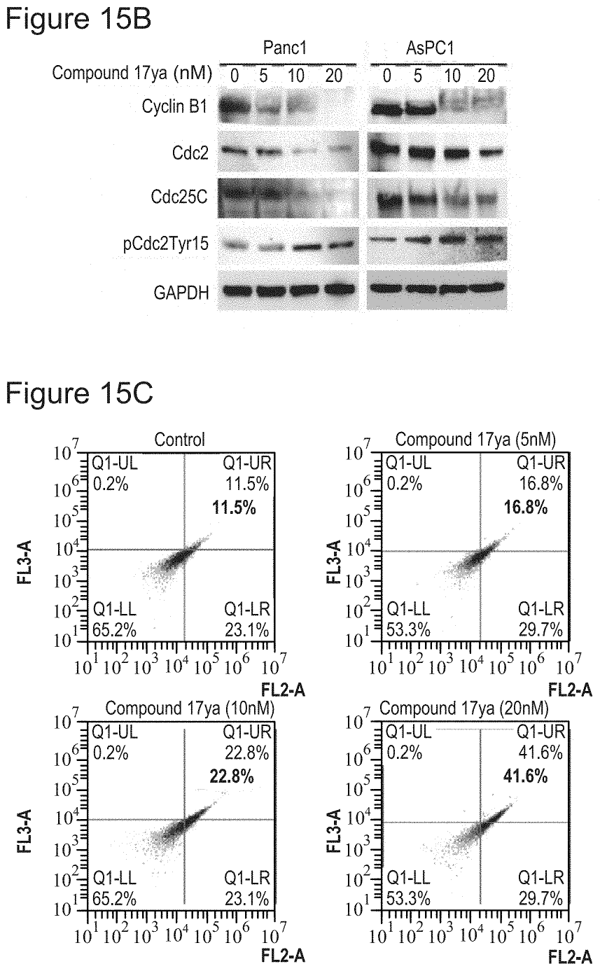

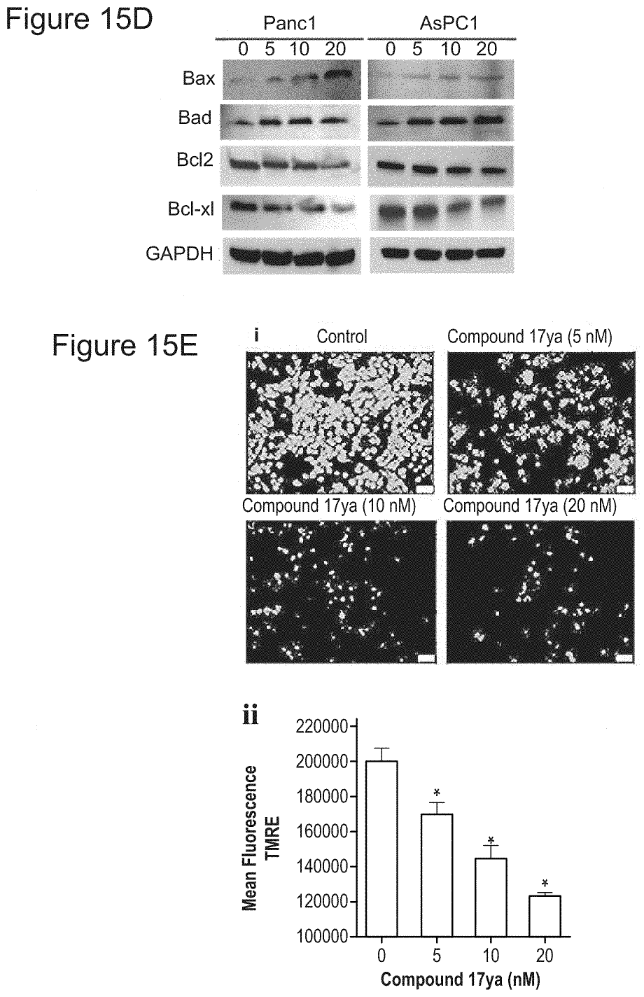

Compound 17ya was studied in a pre-clinical mouse model of pancreatic cancer. Highly aggressive AsPC-1 cells (2×10⁶) were ectopically injected in athymic nude mice to generate xenograft tumors. Compound 17ya (50 μg/mice) and its respective vehicle controls (PBS) were administered intra-tumorally 3 times a week until tumor volume reached 200 mm³ and continued an additional 5 weeks. Compound 17ya effectively inhibited xenograft tumors as compared to vehicle-treated mice as determined by a significant (p=0.01) decrease in tumor volume (FIG. 13A), and tumor weight (FIG. 13B). The average tumor volume in control mice reached the targeted volume of 900 mm³ within 5 weeks. At 5 weeks, the average tumor volume in Compound 17ya treated mice was only 400 mm³. See FIG. 13B for comparison. The observed differences in tumor development were statistically significant (p<0.05) starting at week 3 and continued through week 5. IHC results demonstrated an effective inhibition of PCNA expression in Compound 17ya treated mice as compared to control. The results are illustrated in FIG. 13D. Treatment with Compound 17ya significantly (p<0.05) inhibited protein levels of βIII and βIVb-tubulins as determined by immunohistochemistry. See FIG. 13D. Western blot analysis confirmed the results as illustrated in FIG. 13E. Treatment with Compound 17ya showed similar effects at the mRNA expression of βIII and βIVb-tubulins in xenograft tumor tissues as illustrated in FIGS. 13F and 13G. Compound 17ya induced the expression of miR-200c in excised tumors as determined by qPCR (FIG. 13H) and in situ hybridization (FIG. 13I) assays.

In one embodiment, this invention provides methods for treating pancreatic cancer comprising administering to the subject at least one compound described above and/or an isomer, metabolite, pharmaceutically acceptable salt, pharmaceutical product, tautomer, hydrate, N-oxide, polymorph, or crystal of said compound, or any combination thereof in a therapeutically effective amount to treat the pancreatic cancer.

The invention encompasses a method of treating a subject suffering from pancreatic cancer comprising administering to the subject at least one compound described above, or its isomer, metabolite, pharmaceutically acceptable salt, pharmaceutical product, tautomer, hydrate, N-oxide, polymorph, crystal or any combination thereof in an amount effective to treat pancreatic cancer in the subject. In another embodiment, the compound is compound 12db. In another embodiment, the compound is compound 11cb. In another embodiment, the compound is compound 11fb. In another embodiment, the compound is compound 12da. In another embodiment, the compound is compound 12fa. In another embodiment, the compound is compound 12fb. In another embodiment, the compound is compound 12cb. In another embodiment, the compound is compound 55. In another embodiment, the compound is compound 6b. In another embodiment, the compound is compound 17ya.

A still further aspect of the present invention relates to a method of treating a pancreatic cancerous condition that includes: providing at least one compound described above and then administering an effective amount of the compound to a patient in a manner effective to treat or prevent the pancreatic cancerous condition.

According to one embodiment, the patient to be treated is characterized by the presence of a precancerous condition, and the administering of the compound is effective to prevent development of the precancerous condition into the cancerous condition. This can occur by destroying the precancerous cell prior to or concurrent with its further development into a cancerous state.

According to another embodiment, the patient to be treated is characterized by the presence of a cancerous condition, and the administering of the compound is effective either to cause regression of the cancerous condition or to inhibit growth of the cancerous condition, i.e., stopping its growth altogether or reducing its rate of growth. This preferably occurs by destroying cancer cells, regardless of their location in the patient body. That is, whether the cancer cells are located at a primary tumor site or whether the cancer cells have metastasized and created secondary tumors within the patient body.

As used herein, subject or patient refers to any mammalian patient, including without limitation, humans and other primates, dogs, cats, horses, cows, sheep, pigs, rats, mice, and other rodents. In one embodiment, the subject is male. In another embodiment, the subject is female. In some embodiments, while the methods as described herein may be useful for treating either males or females.

When administering the compounds, they can be administered systemically or, alternatively, they can be administered directly to a specific site where cancer cells or precancerous cells are present. Thus, administering can be accomplished in any manner effective for delivering the compounds or the pharmaceutical compositions to the cancer cells or precancerous cells. Exemplary modes of administration include, without limitation, administering the compounds or compositions orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes, such as, that of the nose, throat, and bronchial tubes.

The method encompasses administering at least one compound in combination with an anti-cancer agent by administering the compounds as herein described, alone or in combination with other agents.

When the compounds or pharmaceutical compositions of the present invention are administered to treat, suppress, reduce the severity, reduce the risk, or inhibit a cancerous condition, the pharmaceutical composition can also contain, or can be administered in conjunction with, other therapeutic agents or treatment regimen presently known or hereafter developed for the treatment of various types of cancer. Examples of other therapeutic agents or treatment regimen include, without limitation, radiation therapy, immunotherapy, chemotherapy, surgical intervention, and combinations thereof.

The following examples are presented to more fully illustrate the preferred embodiments of the invention. They should in no way, however, be construed as limiting the broad scope of the invention.

### EXAMPLES

The Examples set forth below are for illustrative purposes only and are not intended to limit, in any way, the scope of the present invention.

Synthesis of Compound 17ya (ABI-231) was carried out by following a previously reported procedure. Chen et al., “Discovery of novel 2-aryl-4-benzoyl-imidazole (ABI-Ill) analogues targeting tubulin polymerization as antiproliferative agents.” J. Med. Chem., 2012, 55. 7285-7289. Briefly, commercially available indole-3-carboxaldehyde was treated with benzenesulfonyl chloride followed by refluxing with glyoxal and ammonium hydroxide to provide intermediate 1. Intermediate 1 was protected with phenylsulfonyl and subsequently treated with 3,4,5-trimethoxy benzoyl chloride in the presence of tert-butyl lithium to afford compound 2. The final product Compound 17ya was furnished when Compound 2 was refluxed with potassium hydroxide. All intermediates and the final product were fully characterized, as exemplified by proton NMR.

Antibodies and Reagents. MTT (3-(4,5-dimethyl-2-thia-zolyl)-2,5-diphenyl-2-1H-tetrazolium bromide), Phenylmethanesulfonyl fluoride (PMSF), fetal bovine serum (FBS), eukaryotic protease inhibitor cocktail, pyruvic acid and Propidium iodide (PI), were purchased from Sigma-Aldrich Co. (St. Louis, Mo.) or Fisher Scientific (Pittsburgh, Pa.). Mouse anti-human monoclonal antibodies to β tubulins, βIII-tubulin, βIV-tubulin, and rabbit anti-human antibodies to βI-tubulin, βII-tubulin, βIV-tubulin, βV-tubulin, βVI-tubulin, PARP, cyclin B1, Cdc25C, Cdc2, p-Cdc2Tyr15, Bax, Bcl-2, Bad and Bcl-xL were purchased from Cell Signaling Technology. The anti-mouse IgG HRP and rabbit IgG HRP-linked secondary antibodies were procured from Promega (Madison, Wis.). The hematoxylin stain was purchased from Fisher Scientific and the Annexin V/FITC apoptosis kit from Bio-Rad (Hercules, Calif.).

Cell lines. Panc-1, AsPC-1, and HPAF-II cells were obtained from ATCC and cultured in their respective media as DMEM, RPMI-1640 and DMEM/F12 containing 10% FBS and 1% antibiotic/antimycotic. These cells were expanded and frozen aliquots (passage <6) were stored in liquid nitrogen. When needed, cells were thawed and cultured for less than 6 months. Cells were maintained in CO₂ incubator at 37° C. with 98% humidity and 5% CO₂ environment.

Cell Proliferation by MTT Assay. The anti-proliferative effect of Compound 17ya, colchicine, and vinorelbine on pancreatic cancer cells was examined by MTT assay as described by Khan. Kahn et al., Ormeloxifene suppresses desmoplasia and enhances sensitivity of gemcitabine in pancreatic cancer,” Cancer Res., 2015, 75, 2292-2304. Briefly, cells were grown in 96-wells plate at 5×10³ cells per well for 24 hrs and treated with various concentrations of the Compound 17ya (1.25-160 nM) for, 24 and 48 hrs. MTT (5 mg/mL) was added into each well. The plates were incubated for 24 hrs at 37° C. and formazan crystals were dissolved with 100 μL of DMSO. The absorbance at 570 nm was recorded using an OPTImax microplate reader (Molecular Devices; Sunnyvale, Calif.). The cell survival percentages were calculated by dividing the mean optical density (OD) of compound-containing wells by that of DMSO-containing control wells. The IC₅₀ of each compound was calculated by Graph Pad Prism version 5.0.

Colony Forming Assay. For the clonogenic assay, cells were seeded at a density of 250 per well in 12-well plates. Two days after incubation, cells were treated with Compound 17ya (1.25-5 nM) for 12 days. Control cells were treated with DMSO (<0.01%) as a vehicle. Visible colonies (50 cells) were counted following crystal violet staining and results were shown as percent colony formation in each group. Chauhan et al., “MUC13 mucin augments pancreatic tumorigenesis,” Mol. Cancer Ther., 2012, 11, 24-33.

Cell Transfection. The cells were transfected using Lipofectamine, 2000 (Invitrogen) following the manufacturer's protocol. Briefly, Panc-1 and AsPC-1 cells were transiently transfected with miR-200c mimics or non-targeting control mimic (NC) at 100 nM (Applied Biosystems) for 24 and 48 hrs. Cells were pelleted for RNA and cell lysates preparation.

Quantitative Reverse Transcription Polymerase Chain Reactions (qRT-PCR). Total RNA was extracted from control and Compound 17ya treated pancreatic cancer cells using TRIzol™ reagent (Invitrogen, Life Technologies, Grand Island, N.Y.). The integrity of the RNA was checked with an RNA 6000 Nano Assay kit and 2100 Bioanalyzer (Agilent Technologies, Santa Clara, Calif.). cDNAs were prepared by SYBR Green RNA Reverse Transcription kit. The mRNA expression of beta tubulin isotypes was analyzed by qPCR using specific primers sequences as described by Lobert (Lobert et al., “Expression Profiling of Tubulin Isotypes and Microtubule-interacting Proteins using Real-Time Polymerase Chain Reaction,” Methods Cell. Biol., 2010, 95, 47-58. For miRNA detection, 100 ng total RNA was reverse transcribed into cDNA using specific primers designed for miRNA analysis (Applied Biosystems, Foster City, Calif.). Expression of miRNA 200c was determined by qPCR using the Taqman PCR master mix and specific primers designed for the detection of mi-R200c (Applied Biosystems). The expression of miR-200c was normalized with endogenous control (RUN6B).

Western Blot Analysis. Pancreatic cancer cells (1×10⁶) were treated with Compound 17ya, colchicine, and vinorelbine (5-40 nM) for 24 hrs. Total cell lysates were prepared and subjected to Western blot analysis for detecting protein levels of various beta tubulin isoforms and other oncoproteins as described by Khan. Khan et al., “Ormeloxifene Suppresses desmoplasia and Enhances Sensitivity of Gemcitavine in Pancreatic Cancer,” Cancer Res., 2015, 75, 2292-2304.

In situ hybridization. To determine the expression of miR-200c, we performed in situ hybridization in excised tumor tissues of control and Compound 17ya treated mice by Biochain kit (Biochain, San Francisco, Calif.) as described by Khan. Briefly, tissues were hybridized and probed with digoxigenin-labeled miR-200c at 45° C. for overnight incubation. The tissues were subsequently incubated overnight with the AP-conjugated anti-digoxigenin antibody. The slides were mounted, imaged and analyzed under Scan Scope®XT/XT2 system (Aperio, Vista, Calif.).

Cell Migration, Invasion and Motility. Cell migration assay was performed in Corning's 96-well HTS Transwell as per manufacturer's instructions with minor modifications. Cells were treated with Compound 17ya (1.25-10 nM) for 24 hrs. Cells were seeded in the upper chamber with FBS-free media and allowed to migrate towards the lower chamber containing 10% FBS. Cells in the upper chamber were fixed with 4% para-formaldehyde, and with stained with crystal violet. Further, a wound healing assay was also performed to evaluate the effect of Compound 17ya on cell migration. The layer of cells was scraped with a 20-200 μl micropipette tip to create a wound of ^{˜}1 mm width and treated with various concentrations of Compound 17ya. Images of the wounds were monitored under a phase-contrast microscope at 10× magnification. For Invasion assays, a BD Biocoat Matrigel Invasion Chamber (BD Biosciences, Heidelberg, Germany) was incorporated according to the manufacturer's protocol. Then, cells were treated with different concentrations of Compound 17ya and further incubated for 24 hrs. Non-invading cells were removed from the upper surface, invaded cells fixed with cold methanol and stained with crystal violet as described by Chauhan. Chauhan et al., “MUC13 Mucin Augments Pancreatic Tumorigenesis,” Mol. Cancer Ther., 2012, 11, 24-33.

Real Time Cell proliferation, Migration and Invasion by xCELLigence Assays. The effect of Compound 17ya on proliferation, migration and invasion of Panc-1 and AsPC-1 cells was investigated by xCELLigence technology. pancreatic cancer cells were seeded per chamber for cell proliferations (4×10³ for migration, and 4×10⁴ for invasion) in E plates following the xCELLigence real time cell analyzer manuals. Compound 17ya and vehicle control were added at indicated time and concentrations. The baseline cell index for Compound 17ya treated cells compared to control cells was calculated for at least two measurements from three independent experiments.

Cell Cycle Analysis. Effect of Compound 17ya on cell cycle arrest of Panc-1 and AsPC-1 cells was analyzed by flow cytometric analysis. Briefly, approximately 70% confluent cells were synchronized by overnight incubation in FBS free media. Cells were exposed to Compound 17ya (0, 5, 10, 20 and 40 nM) for 24 hrs. The cells were harvested and fixed in ice-cold 70% ethanol overnight followed by incubation with RNAse and incubated with the DNA staining Propidium iodide (Sigma). The DNA content was determined by flow cytometry. Data regarding the number of cells in different phases of the cell cycle was analyzed by BD Accuri C6; Becton-Dickinson, Mountain View, Calif.

Apoptosis. The effect of Compound 17ya on apoptosis induction in pancreatic cancer cells was analyzed by Annexin V-7AAD staining and mitochondrial membrane potential (Δψm). Briefly, pancreatic cancer cells (1×10⁶) were treated with Compound 17ya (5-40 nM) for 24 hrs. These cells were then collected and stained with Annexin V and 7-AAD (5 μl/100 μl of cell suspension). Cells were incubated for 20 min in the dark at room temperature and apoptotic cells were analyzed by Accuri C6 Flow Cytometer setting FL2 and FL3 channels. Effect of Compound 17ya on mitochondrial membrane potential (Δψm) in pancreatic cancer cells was analyzed by uptake of tetramethylrhodamine (TMRE) staining. TMRE is sequestered by active mitochondria and undergoes a dramatic increase in fluorescence intensity. Briefly, pancreatic cancer cells were treated with Compound 17ya (5-20 nM) for 6, 12, and 24 hours, and further incubated with TMRE (100 nM) for 20 min; Fluorescence intensities of TMRE stained cells were measured by flow cytometry. Results were illustrated by mean fluorescence values of TMRE staining of Compound 17ya and vehicle treated control cells.

Xenograft Study. To investigate the therapeutic effects of Compound 17ya against pancreatic cancer, we performed ectopic xenograft studies in athymic nude mice. Six-week-old female athymic nude mice (nu/nu) (n=12) were purchased from Jackson laboratory and maintained in a pathogen-free environment. All procedures were carried out as per the approved UTHSC-IACUC protocol. To establish ectopic xenograft tumors in mice, AsPC-1 cells (2×10⁶ cells) were suspended in 100 μl (1×PBS) and 100 μl Matrigel (BD Biosciences) and were then injected subcutaneously on the dorsal flanks of each mouse. The mice were periodically monitored for tumor growth using a digital vernier caliper. Mice were divided into two groups (Control (n=6) and Compound 17ya (n=6)), when their tumor volume were reached ^{˜}200 mm3. Mice were administered with Compound 17ya (50 μg/mouse) intra-tumorally, and control mice administered with vehicle controls (1×PBS) were injected. Tumor volumes were measured weekly and calculated by the formula 0.5238×L×W×H, where L is length, W is width and H is the height of the tumor. All of the mice were euthanized when tumors of the control group mice reached the targeted volume of ^{˜}1000 mm³. Tumors of both group's mice were excised and used for RNA, tissue lysates, histopathology and slides preparation (5 μm section).

Immunohistochemistry. The effect of Compound 17ya was determined on the expression of PCNA and tubulin isoforms in excised xenograft tumors by immunohistochemistry, using kits from Biocare (Biocare Medical, Concord, Calif.) as described previously. XAX what does this reference mean? (44).

Statistical Analysis. The data discussed above are presented in terms of mean values and the S.E.M. of several independent experiments. p-values <0.05 were considered statistically significant. All statistical analyses were performed using the Statistical Package for the Social Sciences, version 11.5 (SPSS Inc., Chicago, Ill.).

### Example 1

The results determined that Compound 17ya inhibited growth and clonogenic potential of pancreatic cancer cells. The cytotoxic effect of Compound 17ya against various human pancreatic cancer cell lines (AsPC-1, Panc-1 and HPAF-II) was determined. In the experiment, cells were treated with various concentrations of Compound 17ya (1.25-160 nM) for 24 and 48 hrs, and cell viability was determined by MTT assay. Compound 17ya inhibited the growth of pancreatic cancer cells in a dose- and time-dependent manner. The results are illustrated in FIG. 1A and FIG. 1B. After 24 hours of treatment, the IC₅₀ of Compound 17ya was 20, 30 and 30 nM in Panc-1, AsPC-1 and HPAF-II respectively, (FIG. 1A), while 48 hrs post-treatment the IC₅₀ was 8.2, 12.5, and 20 nM, respectively (FIG. 1B). The growth inhibitory effect of Compound 17ya in real time with the xCELLigence system was analyzed. The system monitors cell growth by measuring electrical impedance, which is expressed as a cell index. A growth curve, recorded as the basal cell index value, showed that Compound 17ya significantly reduced the cell index in a dose-dependent manner compared to vehicle treated pancreatic cancer cells. The results are illustrated in FIGS. 2A and 2B. To determine the long-term effect of Compound 17ya on the growth of pancreatic cancer cells. Compound 17ya (1.25-5 nM) treatment significantly reduced the number of colonies in a dose-dependent manner in Panc-1 (FIG. 3A), AsPC-1 (FIG. 3B) and HPAF-II (FIG. 3C) cells as compared to respective control groups.

### Example 2. Compound 17ya Inhibited mRNA Expression and Protein Stability of β-Tubulin Isotypes in Pancreatic Cancer Cells

Compound 17ya inhibited mRNA expression and protein stability of β-tubulin isotypes in pancreatic cancer cells. Compound 17ya (5-20 nM) treatment significantly (p<0.01) inhibited the mRNA expression of βIII and βIV-tubulins in a dose-dependent manner in both Panc-1 and AsPC-1 cells (FIG. 4A) as determined by qRT-PCR. The effect of Compound 17ya on these tubulins at translational level was determined. Western blot analysis results demonstrated that Compound 17ya inhibited protein levels of βIII and βIV-tubulins in both Panc-1 and AsPC-1 (FIG. 4B) cells. The effect of Compound 17ya on other tubulin isotypes to determine its specificity at both the mRNA and protein level was studied. Compound 17ya inhibited the mRNA and protein expression of βI-tubulins in Panc-1 and AsPC-1 cells (FIGS. 4A and 4B). However, no effect was observed on either mRNA or protein for βIIa, βIIb and βV-tubulins in any tested pancreatic cancer cells (FIGS. 4A and 4B). The effect of Compound 17ya on the expression of βIII-tubulin with known β-tubulin-destabilizers (colchicine, and vinorelbine) was studied. In the experiment Panc-1 cells were treated with 5-40 nM of Compound 17ya, colchicine, and vinorelbine for 24 hrs, and RNA and protein lysates were prepared to determine the mRNA expression and protein level of βIII-tubulin. In comparison to colchicine, Compound 17ya more effectively inhibited mRNA expression (FIG. 5A) and protein levels (FIG. 5B) of βIII-tubulin. The functional impact of Compound 17ya with colchicine and vinorelbine in Panc-1, AsPC-1, and HPAF-II cells by performing MTT assays was determined. Compound 17ya showed the most effective cell growth inhibition in all pancreatic cancer cell lines as compared to colchicine and vinorelbine as illustrated in FIGS. 6A, 6B, and 6C.

### Example 3. Compound 17ya Restored the Expression of miR-200c Via Targeting Pill-Tubulin

Compound 17ya restored the expression of miR-200c via targeting βIII-tubulin. The underlying molecular mechanism of Compound 17ya targeting βIII-tubulin in pancreatic cancer cells was studied. It was reported that miR-200c directly targets βIII-tubulin in pancreatic cancer cells. Cochrane et al., “MicroRNA-200c Mitigates Invasiveness and Restores Sensitivity to Microtule-Targeting Chemotherapeutic Agents,” Mol. Cancer Ther., 2009, 8, 1055-1066. Compound 17ya treatment induced the expression of miR-200c in Panc-1 (FIG. 7A) and AsPC-1 (FIG. 7B) when compared with control cells. We determined whether inhibition of miR-200c minimized the effect of Compound 17ya on the expression of βIII tubulin. Transfection of miR-200c mimicked in Panc-1 cells inhibited the expression of βIII-tubulin, which was rescued by transfecting the cells with miR-200c inhibitor (FIG. 7B). Compound 17ya treatment and miR-200c mimic transfection of Panc-1 cells showed a synergistic effect on the expression of βIII tubulin at both mRNA (FIG. 7B) and protein (FIG. 7C) levels. The results demonstrated that Compound 17ya inhibited the expression of βIII tubulin via restoring the expression of miR-200c in pancreatic cancer cells.

### Example 4. Compound 17ya Inhibited Migration and Invasive Potential of Pancreatic Cancer Cells

Compound 17ya inhibited migration and invasive potential of pancreatic cancer cells. We determined whether Compound 17ya targeted β-tubulins and the effect on the invasive and migratory potential of pancreatic cancer cells. Wound healing assays determined the effect of Compound 17ya on the migration of pancreatic cancer cells. The results revealed remarkable inhibition in migration of Panc-1 (FIG. 8A), AsPC-1 (FIG. 8B), and HPAF-II cells when treated with sub-lethal concentrations of Compound 17ya (1.25 and 2.5 nM). The effect of Compound 17ya on pancreatic cancer cell migration by transwell assay was also evaluated. Compound 17ya (1.25-2.5 nM) showed significant (p<0.01) inhibition of Panc-1 (FIG. 9A), AsPC-1 (FIG. 9B) and HPAF-II cell migration in a dose-dependent manner. Compound 17ya at sub lethal concentrations (1.25-2.5 nM) also significantly (p<0.01) inhibited invasion of Panc-1 (FIG. 10A), AsPC-1 (FIG. 10B) and HPAF-II cells as compared to the vehicle treatment group. The effect of Compound 17ya on migration and invasion of pancreatic cancer cells was further confirmed using the xCELLigence system. Compound 17ya also dose-dependently (5-20 nM) reduced the baseline cell index of pancreatic cancer cells as compared to control, which reflects potent inhibitory effects of Compound 17ya on pancreatic cancer cells migration (FIG. 11A) and invasion (FIG. 11B).

### Example 5. Compound 17ya Arrested Cell Cycle in G2/M Phase and Induced Apoptosis in Pancreatic Cancer Cells

Compound 17ya arrests cell cycle in G2/M phase and induces apoptosis in pancreatic cancer cells. Compound 17ya destabilized β-tubulins and inhibited their polymerization, this study evaluated its effect on pancreatic cancer cell cycle distribution. The effect of Compound 17ya on cell cycle distribution of pancreatic cancer cells was examined by flow cytometry. Compound 17ya treatment arrested the cell cycle of Panc-1 (FIG. 12A) and AsPC-1 cells in G2/M phase in a dose-dependent manner. The effect of Compound 17ya on cell cycle regulatory proteins was investigated. The complex formation between cdc2 and cyclin B1 is an important event for cell entry into mitosis. As shown in FIG. 12B, Compound 17ya dose-dependently inhibited the protein levels of cyclin B1 and cdc25c in Panc-1 and AsPC-1 cells. Compound 17ya also dose-dependently (5-20 nM) inhibited both the phosphorylation and total protein of cyclin-dependent kinase Cdc2, in Panc-1 and AsPC-1 cells (FIG. 12B). Given the observed arrest of cell cycle in G2/M phase, the effect of Compound 17ya on apoptosis induction in pancreatic cancer cells was investigated using Annexin V-7AAD staining and mitochondrial membrane potential (Δψm) using flow cytometer. As shown in FIG. 12C, Compound 17ya treatment (5-20 nM) resulted in apoptosis induction in both Panc-1 (FIG. 12C) and AsPC-1 cells. Compound 17ya treatment (10-20 nM) illustrated 22.8% and 41.6% apoptotic population of Panc-1 cells (FIG. 12C), whereas AsPC-1 cells showed 11.5% and 18.0% apoptotic cells, respectively, at same concentrations. The effect of Compound 17ya on Δψm in Panc-1 and AsPC-1 cells using TMRE staining was studied. Compound 17ya illustrated a dose-dependent (5-20 nM) decrease of TMRE staining in both Panc-1 (FIG. 12E) and AsPC-1 cells. The effect of Compound 17ya on other mitochondrial pro-apoptotic (Bax and Bad) and anti-apoptotic (Bcl2 and Bcl-xL) proteins was studied. Compound 17ya (5-20 nM) induced the expression of Bax and Bad, and inhibited expression of Bcl-2 and Bcl-xl proteins (FIG. 12D). The results suggested that Compound 17ya arrested the cell cycle in the G2/M phase and induced apoptosis via intrinsic mechanism in pancreatic cancer cells.

### Example 6. Compound 17ya Effectively Inhibited the Growth of Pancreatic Tumors in a Xenograft Mouse Model

Compound 17ya effectively inhibited the growth of pancreatic tumors in a xenograft mouse model. The therapeutic effect of Compound 17ya in a pre-clinical mouse model of pancreatic cancer was studied. In this experiment, highly aggressive AsPC-1 cells (2×10⁶) were ectopically injected in athymic nude mice to generate xenograft tumors. Compound 17ya (50 μg/mice) and its respective vehicle controls (PBS) were administered intra-tumorally 3 times a week until tumor volume reached 200 mm³ and continued an additional 5 weeks. Compound 17ya treatment effectively inhibited xenograft tumors as compared to vehicle-treated mice as determined by a significant (p=0.01) decrease in tumor volume (FIG. 13A and FIG. 13B), and tumor weight (FIG. 13C). The average volume of tumors in control mice reached the targeted volume of 900 mm³ within 5 weeks. At this time, the average tumor volume in Compound 17ya-treated mice was only 400 mm³ (FIG. 13B). There was a significant interaction between treatment and time, so differences were tested over time. The observed differences in tumor development was statistically significant (p<0.05) starting at week 3 and continued through week 5. PCNA is one of the markers of cell proliferation, which is aberrantly upregulated in cancer cells. IHC results demonstrated an effective inhibition of PCNA expression in Compound 17ya treated mice as compared to control (FIG. 13D). Because Compound 17ya potentially targeted βIII and βIVa and βIVb-tubulins in pancreatic cancer cells in in vitro, these findings were translated to an in vivo system. The expression of these tubulins in excised xenograft tumors from vehicle and Compound 17ya treated mice was studied. Compound 17ya treatment significantly (p<0.05) inhibited protein levels of βIII and βIVb-tubulins as determined by immunohistochemistry (FIG. 13D). These results were further confirmed by Western blot analysis (FIG. 13E). Compound 17ya showed similar effects at the mRNA expression of βIII and βIVb-tubulins in xenograft tumor tissues (FIG. 13F and FIG. 13G). Compound 17ya also induced the expression of miR-200c in excised tumors as determined by qPCR (FIG. 13H) and in situ hybridization (FIG. 13I) assays.

All of the features described herein (including any accompanying claims, abstract and drawings), and/or all of the steps of any method or process so disclosed, may be combined with any of the above aspects in any combination, except combinations where at least some of such features and/or steps are mutually exclusive. Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of treating pancreatic cancer in a subject in need thereof comprising administering to the subject a therapeutically effective amount of a compound represented by the structure of formula XI: 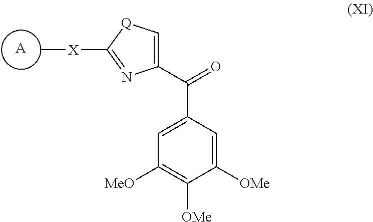
wherein
X is a bond, NH or S;
Q is O, NH or S; and
A is substituted or unsubstituted single-, fused- or multiple-ring, aryl or (hetero)cyclic ring systems;
substituted or unsubstituted, saturated or unsaturated N-heterocycles; substituted or unsubstituted, saturated or unsaturated S-heterocycles; substituted or unsubstituted, saturated or unsaturated O-heterocycles; substituted or unsubstituted, saturated or unsaturated cyclic hydrocarbons; or substituted or unsubstituted or saturated or unsaturated mixed heterocycles;
wherein said A ring is optionally substituted by 1-5 substituents which are independently O-alkyl, O-haloalkyl, F, Cl, Br, I, haloalkyl, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, alkylamino, aminoalkyl, —OCH₂Ph, —NHCO-alkyl, COOH, —C(O)Ph, C(O)O-alkyl, C(O)H, —C(O)NH₂ or NO₂; and
i is an integer between 0-5;
wherein if Q is 5, then X is not a bond, or an isomer, pharmaceutically acceptable salt, pharmaceutical product, tautomer, hydrate, N-oxide, or combinations thereof, to treat the pancreatic cancer.

2. The method according to claim 1, wherein said compound is represented by the structure of formula VIII: 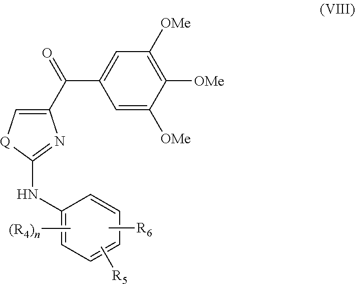
R₄, R₅ and R₆ are independently hydrogen, O-alkyl, O-haloalkyl, F, Cl, Br, I, haloalkyl, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, alkylamino, aminoalkyl, —OCH₂Ph, —NHCO-alkyl, COOH, —C(O)Ph, C(O)O-alkyl, C(O)H, —C(O)NH₂ or NO₂;
Q is S, O or NH;
i is an integer between 0-5; and
n is an integer between 1-3.

3. The method according to claim 1, wherein said compound is represented by the structure of formula XI(b): 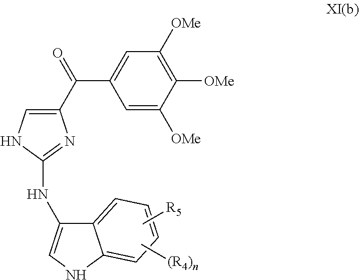
wherein R₄ and R₅ are independently hydrogen, O-alkyl, O-haloalkyl, F, Cl, Br, I, haloalkyl, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4.

4. The method according to claim 1, wherein said compound is represented by the structure of formula XI(c): 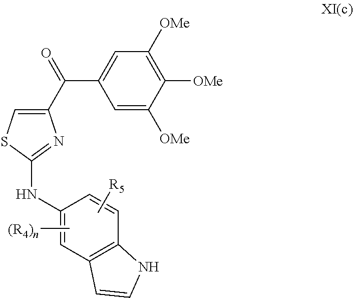
wherein R₄ and R₅ are independently hydrogen, O-alkyl, O-haloalkyl, F, Cl, Br, I, haloalkyl, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4.

5. The method according to claim 4, wherein said compound is compound 55, represented by the structure: 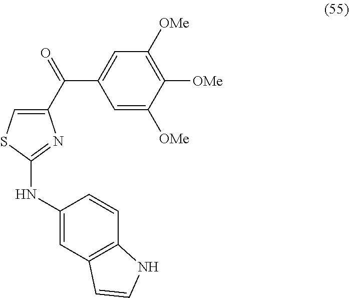 [SMILES: COc1cc(C(=O)c2csc(Nc3ccc4[nH]ccc4c3)n2)cc(OC)c1OC]
or a pharmaceutically acceptable salt.

6. The method according to claim 2, wherein said compound is (2-(phenylamino)thiazol-4-yl)(3,4,5-trimethoxyphenyl)methanone (5a), (2-(p-tolylamino)thiazol-4-yl)(3,4,5-trimethoxyphenyl)methanone (5b), (2-(p-fluorophenylamino)thiazol-4-yl)(3,4,5-trimethoxyphenyl)methanone (5c), (2-(4-chlorophenylamino)thiazol-4-yl)(3,4,5-trimethoxyphenyl)methanone (5d), or (2-(phenylamino)-1H-imidazol-4-yl)(3,4,5-trimethoxyphenyl)methanone (5e).

7. The method according to claim 1, wherein the compound is combined with a pharmaceutically acceptable carrier.

8. The method according to claim 1, further comprising administering an additional cancer therapy.

9. A method of treating pancreatic cancer in a subject in need thereof comprising administering to the subject a therapeutically effective amount of a compound represented by the structure of formula XI(e): 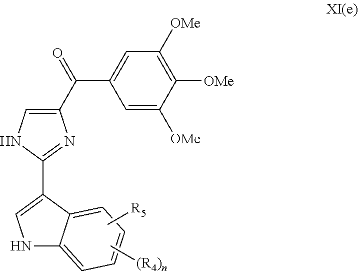
wherein R₄ and R₅ are independently hydrogen, O-alkyl, O-haloalkyl, F, Cl, Br, I, haloalkyl, CN, —CH₂CN, NH₂, hydroxyl, —(CH₂)ᵢNHCH₃, —(CH₂)ᵢNH₂, —(CH₂)ᵢN(CH₃)₂, —OC(O)CF₃, C₁-C₅ linear or branched alkyl, alkylamino, aminoalkyl, —OCH₂Ph, —NHCO-alkyl, COOH, —C(O)Ph, C(O)O-alkyl, C(O)H, —C(O)NH₂ or NO₂;
i is an integer from 0-5; and
n is an integer between 1-4, or an isomer, pharmaceutically acceptable salt, pharmaceutical product, tautomer, hydrate, N-oxide, or combinations thereof, to a treat the pancreatic cancer.

10. The method according to claim 9, wherein said compound is compound 17ya represented by the structure: 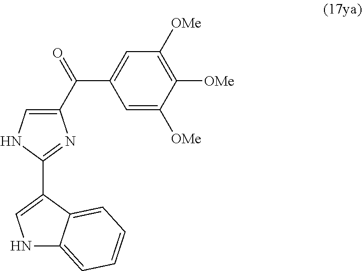 [SMILES: COc1cc(C(=O)c2c[nH]c(-c3c[nH]c4ccccc34)n2)cc(OC)c1OC]
or a pharmaceutically acceptable salt thereof.

11. The method according to claim 9, further comprising an additional cancer therapy.
